SEQUENCE LISTING (72 sequences)

SEQ ID NO: 1 - protein, 21 aa, synthetic construct
MALPVTALLLPLALLLHAARP

SEQ ID NO: 2 - protein, 19 aa, synthetic construct
MALPVTALLLPLALLLHAA

SEQ ID NO: 3 - protein, 11 aa, synthetic construct
MALPVTALLLP

SEQ ID NO: 4 - protein, 12 aa, synthetic construct
PVTALLLPLALL

SEQ ID NO: 5 - protein, 14 aa, synthetic construct
LLLPLALLLHAARP

SEQ ID NO: 6 - protein, 45 aa, synthetic construct
TTTPAPRPPTPAPTIASQPLSLRPEACRPAAGGAVHTRGLDFACD

SEQ ID NO: 7 - protein, 27 aa, synthetic construct
FWVLVVVGGVLACYSLLVTVAFIIFWV

SEQ ID NO: 8 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 9 - protein, 42 aa, synthetic construct
KRGRKKLLYIFKQPFMRPVQTTQEEDGCSCRFPEEEEGGCEL

SEQ ID NO: 10 - protein, 41 aa, synthetic construct
RSKRSRLLHSDYMNMTPRRPGPTRKHYQPYAPPRDFAAYRS

SEQ ID NO: 11 - protein, 116 aa, synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 12 - protein, 40 aa, synthetic construct
CVRCRHRRRQAERMSQIKRLLSEKKTCQCPHRFQKTCSPI

SEQ ID NO: 13 - protein, 32 aa, synthetic construct
LYCNHRNRRRVCKCPRPVVKSGDKPSLSARYV

SEQ ID NO: 14 - protein, 19 aa, synthetic construct
HLCCRRRRARLRFMKQFYK

SEQ ID NO: 15 - protein, 235 aa, synthetic construct
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 16 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 22 aa, synthetic construct
GSGATNFSLLKQAGDVEENPGP

SEQ ID NO: 18 - protein, 20 aa, synthetic construct
GGGGSGGGGSGGGGSGGGGS

SEQ ID NO: 19 - protein, 15 aa, synthetic construct
GGGGSGGGGSGGGGS

SEQ ID NO: 20 - protein, 10 aa, synthetic construct
GGGGSGGGGS

SEQ ID NO: 21 - protein, 5 aa, synthetic construct
GGGGS

SEQ ID NO: 22 - protein, 308 aa, synthetic construct
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 23 - protein, 313 aa, synthetic construct
VGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 24 - protein, 330 aa, synthetic construct
GSGATNFSLLKQAGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 25 - protein, 335 aa, synthetic construct
GSGATNFSLLKQAGDVEENPGPVGPFEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSP

SEQ ID NO: 26 - protein, 710 aa, synthetic construct
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 27 - protein, 120 aa, synthetic construct
EVKLEESGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSS

SEQ ID NO: 28 - protein, 108 aa, synthetic construct
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKR

SEQ ID NO: 29 - protein, 121 aa, synthetic construct
EVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSS

SEQ ID NO: 30 - protein, 113 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIER

SEQ ID NO: 31 - protein, 121 aa, synthetic construct
GSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 32 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 33 - protein, 122 aa, synthetic construct
EVQLVESGGGLVQPGGSLRLSCAASGYSFTGYTMNWVRQAPGKCLEWVALINPYKGVSTYNQKFKDRFTISVDKSKNTAYLQMNSLRAEDTAVYYCARSGYYGDSDWYFDVWGQGTLVTVSS

SEQ ID NO: 34 - protein, 107 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGCGTKVEIK

SEQ ID NO: 35 - protein, 116 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVS

SEQ ID NO: 36 - protein, 108 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 37 - protein, 119 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 38 - protein, 112 aa, synthetic construct
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 39 - protein, 122 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 40 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 41 - protein, 118 aa, synthetic construct
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 42 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 43 - protein, 120 aa, synthetic construct
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 44 - protein, 107 aa, synthetic construct
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 45 - protein, 120 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSG

SEQ ID NO: 46 - protein, 115 aa, synthetic construct
GSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 47 - protein, 153 aa, synthetic construct
LKPREVKLVESGGGLVQPGGSLKLSCAASGFDFSRYWMSWVRQAPGKGLEWIGEINLDSSTINYTPSLKDKFIISRDNAKNTLYLQMSKVRSEDTALYYCARRYDAMDYWGQGTSVTVSSAKTTAPSVYPLAPVCGDTTGSSVTLGCLVKASQ

SEQ ID NO: 48 - protein, 111 aa, synthetic construct
ASDIVLTQSPASLAVSLGQRATISCRASESVDDYGISFMNWFQQKPGQPPKLLIYAAPNQGSGVPARFSGSGSGTDFSLNIHPMEEDDTAMYFCQQSKDVRWRHQAGDQTG

SEQ ID NO: 49 - protein, 485 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 50 - protein, 198 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSITCPPPMSVEHADIWVKSYSLYSRERYICNSGFKRKAGTSSLTECVLNKATNVAHWTTPSLKCIR

SEQ ID NO: 51 - protein, 11 aa, synthetic construct
LASQTIDTWLA

SEQ ID NO: 52 - protein, 7 aa, synthetic construct
AATNLAD

SEQ ID NO: 53 - protein, 9 aa, synthetic construct
QQVYSSPFT

SEQ ID NO: 54 - protein, 23 aa, synthetic construct
DIQMTQSPASLSASVGDRVTITC

SEQ ID NO: 55 - protein, 15 aa, synthetic construct
WYLQKPGKSPQLLIY

SEQ ID NO: 56 - protein, 32 aa, synthetic construct
GVPSRFSGSGSGTDFSFTISSLQPEDFATYYC

SEQ ID NO: 57 - protein, 10 aa, synthetic construct
FGQGTKLEIK

SEQ ID NO: 58 - protein, 106 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTGQGTKLEIK

SEQ ID NO: 59 - protein, 5 aa, synthetic construct
DYNVY

SEQ ID NO: 60 - protein, 17 aa, synthetic construct
YIDPYNGITIYDQNFKG

SEQ ID NO: 61 - protein, 17 aa, synthetic construct
YIDPYNGITIYDQNLKG

SEQ ID NO: 62 - protein, 8 aa, synthetic construct
DVTTALDF

SEQ ID NO: 63 - protein, 30 aa, synthetic construct
QIQLVQSGGEVKKPGASVRVSCKASGYSFT

SEQ ID NO: 64 - protein, 14 aa, synthetic construct
WVRQSPGKGLEWIG

SEQ ID NO: 65 - protein, 32 aa, synthetic construct
KATLTVDKSTSTAYMELSSLRSEDTAVYFCAR

SEQ ID NO: 66 - protein, 11 aa, synthetic construct
WGQGTTVTVSS

SEQ ID NO: 67 - protein, 117 aa, synthetic construct
QIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 68 - protein, 117 aa, synthetic construct
QIQLVQSGGEVKKPGASVRVSCKASGYSFTDYNVYWVRQSPGKGLEWIGYIDPYNGITIYDQNLKGKATLTVDKSTSTAYMELSSLRSEDTAVYFCARDVTTALDFWGQGTTVTVSS

SEQ ID NO: 69 - protein, 107 aa, synthetic construct [sequence omitted]

SEQ ID NO: 70 - protein, 381 aa, synthetic construct
RTVAAPSVFIFPPSDEQLKSGTASVVCLLNNFYPREAKVQWKVDNALQEFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGCFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 474 aa, synthetic construct
YFGKLESKLSVIRNLNMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 72 - protein, 583 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASITCPPPMSVEHADIWVKSYSLYSRERYICNSGFKRKAGTSSLTECVLNKATNVAHWTTPSLKCIR

(19) United States
(12) Patent Application Publication
(10) Pub. No.: US 2024/0374639 A1
(43) Pub. Date: Nov. 14, 2024

(54) EXPANSION OF MEMORY NATURAL KILLER CELLS

(71) Applicant: WUGEN, Inc., St. Louis, MO (US)

(72) Inventors: Ryan Patrick SULLIVAN, Ballwin, MO (US); Kenneth M. CHROBAK, St. Louis, MO (US); Matthew COOPER, St. Louis, MO (US); Mary Elizabeth MATHYER, St. Louis, MO (US); Jennifer L. GOVERO, St. Louis, MO (US)

(21) Appl. No.: 18/412,163

(22) Filed: Jan. 12, 2024

Related U.S. Application Data
(63) Continuation of application No. PCT/US2022/037178, filed on Jul. 14, 2022.
(60) Provisional application No. 63/222,306, filed on Jul. 15, 2021.

(51) Int. Cl. A61K 35/17; A61K 39/00; A61P 35/02; C12N 5/0783

(52) U.S. Cl. CPC A61K 35/17; A61K 39/4613; A61K 39/4631; A61P 35/02; C12N 5/0646; A61K 2239/17; A61K 2239/21; C12N 2501/2312; C12N 2501/2315; C12N 2501/2318

(57) ABSTRACT

The present disclosure generally relates to, inter alia, natural killer (NK) cells including memory-like and cytokine-induced memory like (CIML) NK cells, methods of making and using them e.g. in the treatment of cancer, increasing anti-tumor properties of NK cells.

This application is a Bypass Continuation of International Application Number PCT/US2022/037178, filed Jul. 14, 2022, which claims priority to, and the benefit of, U.S. Provisional Patent Application No. 63/222,306, filed Jul. 15, 2021, the disclosures of each are incorporated by reference as if written herein in their entireties.

### INCORPORATION OF SEQUENCE LISTING

The sequence listing that is contained in the file named “WGN0022-201BC1-US,” which is 71,168 bytes as measured in Microsoft Windows operating system and was created on Jan. 9, 2024, is filed electronically herewith and incorporated herein by reference.

The present disclosure generally relates to, inter alia, natural killer (NK) cells, including memory/memory-like and cytokine-induced memory like (CIML) NK cells, methods of making and using them, e.g., in the treatment of cancer, and increasing anti-tumor properties of NK cells.

Natural killer (NK) cells constitute a group of innate immune cells, which are often characterized as cytotoxic lymphocytes that exhibit antibody dependent cellular toxicity via target-directed release of granzymes and perforin. Most NK cells have a specific cell surface marker profile (e.g., CD3, CD56+, CD16+, CD57+, CD8+) in addition to a collection of various activating and inhibitory receptors. While more recently NK cells have become a significant component of certain cancer treatments, generation of significant quantities of NK cells has been a significant obstacle as the fraction of NK cells in whole blood is relatively low.

Various methods of generating memory NK cells are known in the art, all or almost all of them suffer from various disadvantages, such as low yields, the use of feeder cells, and expensive reagents. Consequently, there is a need to provide improved systems and methods that produce memory NK cells in significant quantities.

Disclosed herein are compositions and methods that enable generation and expansion of memory/memory-like NK cells in a conceptually simple and efficient manner. Memory NK cells can be generated in a process in which NK cells are concurrently primed to form the memory NK cells and expanded to a desired quantity. Alternatively, the NK cells are expanded to a desired quantity and then primed to form the memory NK cells.

### BRIEF DESCRIPTION OF THE SEQUENCES

SEQ ID NOs: 1-48 in tables 4 and 5 are sequences of various components of chimeric antigen receptors.

SEQ ID NOs: 49-50 are sequences of exemplary expansion fusion protein (EFP) chains making up EFP 7t15-21s.

SEQ ID NOs: 51-70 are sequences of exemplary crosslinking agent anti-tissue factor antibody ATF1.

SEQ ID NOs: 71-72 are sequences of exemplary priming fusion protein (PFP) chains making up PFP 18t15-12s.

### BRIEF DESCRIPTION OF THE DRAWINGS

FIG. 1 shows percent cancer cell (K562) killing in vitro on days 6/7 by memory NK cells, at a given effector to target cell ratio, for memory NK cells produced by the given combination of expanding agent and priming agent (7t15-21s+ATF1, 18t15-12s).

FIG. 2 shows percent cancer cell (K562) killing in vitro on day 13 by memory NK cells, at a given effector to target cell ratio, for memory NK cells produced by the given combination of expanding agent and priming agent (7t15-21s+ATF1, 18t15-12s).

FIG. 3 shows percent cancer cell (K562) killing in vitro on day 17 by memory NK cells, at a given effector to target cell ratio, for memory NK cells produced by the given combination of expanding agent and priming agent (7t15-21s+ATF1, 18t15-12s).

FIG. 4 shows the EC50 in cellular ratio of cancer cell (K562) killing by memory NK cells produced by the given combination of expanding agent and priming agent (7t15-21s+ATF1, 18t15-12s).

FIG. 5 shows the cumulative fold change in the number of NK cells stimulated for the given number of days in culture at the given cellular density.

FIG. 6 shows percent cancer cell (K562) killing in vitro at the given effector:target ratio for expanded cells primed with 250 nM of priming agent for the given length of time.

FIG. 7 shows percent cancer cell (K562) killing in vitro at the effector:target ratio of 20:1, on days 2, 4, 6, 8, 10, 12, 14 and 16, for expanded cells primed with 250 nM priming agent 18t15-12s for the given length of time.

FIG. 8 shows percent cancer cell (K562) killing in vitro at the effector:target ratio of 4:1, on days 2, 4, 6, 8, 10, 12, 14 and 16, for expanded cells primed with 250 nM priming agent 18t15-12s for the given length of time.

FIG. 9 shows percent cancer cell (K562) killing in vitro at the effector:target ratio of 0.8:1, on days 2, 4, 6, 8, 10, 12, 14 and 16, for expanded cells primed with 250 nM priming agent 18t15-12s for the given length of time.

FIG. 10 shows percent cancer cell (K562) killing in vitro at the effector:target ratio of 0.16:1, on days 2, 4, 6, 8, 10, 12, 14 and 16, for expanded cells primed with 250 nM priming agent 18t15-12s for the given duration.

FIG. 11 shows IFNg production in cell cultures (K562 only, NK cells that were expanded, and NK cells that were expanded and primed with 250 nM priming agent 18t15-12s for the given durations) after 24 hours of culture either alone or in the presence of K562 target cells

FIG. 12 shows fold expansion of NK cells produced by expansion only, priming then expansion, and expansion then priming.

FIG. 13 shows percent cancer cell (K562) killing in vitro at the given effector: target ratio for NK cells that were isolated, primed for 3 hours, primed overnight, primed overnight then expanded, expanded then primed for 3 hours, expanded then primed overnight, or expanded only.

FIG. 14 shows the Cellular K562 Killing EC₅₀ of NK cells that were isolated, primed for 3 hours, primed overnight, primed overnight then expanded, expanded then primed for 3 hours, expanded then primed overnight, or expanded only.

FIG. 15 shows IFNg production in cell cultures (NK cells alone, or with K562 cells) from NK cells that were: isolated, primed for 3 hours, primed overnight, primed overnight then expanded, expanded then primed for 3 hours, expanded then primed overnight, or expanded only.

FIG. 16 shows fold change, over background on day 7 after injection at t=0, in the number of NK cells produced by having been primed for 3 hours, primed overnight, primed overnight then expanded, expanded only, or expanded then primed for 3 hours, in the blood of immunodeficient NSG mice.

FIG. 17 shows K562-Luc killing on day 14 by expanded and expanded-then-primed NK cells.

FIG. 18 shows the EC50 for K562-Luc killing by expanded and expanded-then-primed NK cells.

### DETAILED DESCRIPTION

Provided herein are compositions and methods that enable generation and expansion of memory/memory-like NK cells in a conceptually simple and efficient manner. Memory NK cells can be generated in a process in which NK cells are concurrently primed to form the memory NK cells and expanded to a desired quantity. Alternatively, the NK cells are expanded to a desired quantity and then primed to form the memory NK cells.

Accordingly, provided herein are memory natural killer (NK) cells produced by, sequentially:
a) expanding a population of purified NK cells; and
b) priming the NK cells.

Also provided herein are purified memory natural killer (NK) cells produced by concurrently priming and expanding a population of purified NK cells.

Also provided herein are memory natural killer (NK) cells produced by, sequentially:
a) purifying a population of NK cells;
b) expanding the NK cells; and
c) priming the NK cells.

Also provided herein are memory natural killer (NK) cells produced by:
a) purifying a population of NK cells; and
b) concurrently priming and expanding the NK cells.

Further disclosed herein is a method of making memory NK cells comprising:
a) expanding a purified population of NK cells; and then
b) priming the NK cells.

Further disclosed herein is a method of making memory NK cells comprising concurrently priming and expanding a purified population of NK cells.

Further disclosed herein is a method of making memory NK cells comprising:
a) purifying a population of NK cells;
b) expanding the NK cells; and then
c) priming the NK cells.

Further disclosed herein is a method of making memory NK cells comprising:
a) purifying a population of NK cells; and
b) concurrently priming and expanding the NK cells.

Also provided are the following embodiments.

In some embodiments, the NK cell population is purified starting from donor blood, or fresh or previously cryopreserved leukapheresate. In some embodiments, the purification is performed via positive selection (for example on the Miltenyi CliniMACS Prodigy). In some embodiments, the purification is performed via negative selection (for example, the StemCell EasySep NK Cell Enrichment Kit). In some embodiments, purification is performed using a combination of positive and negative selection. In some embodiments, the NK cells are differentiated from lymphoid progenitor cells.

In some embodiments, the NK cells are expanded by exposure to an expansion agent comprising a combination of cytokines, or functional fragments thereof, and/or fusion proteins comprising functional fragments thereof, or a combination of any of the foregoing, and optionally a crosslinking agent.

In some embodiments, the NK cells are expanded by exposure to an expansion agent comprising:
one or more of IL-2, IL-4, IL-7, IL-9, IL-15, and IL-21, or functional fragments thereof; or
fusion proteins comprising functional fragments one or more of IL-2, IL-4, IL-7, IL-9, IL-15, and IL-21;
   and optionally a crosslinking agent; or
microspheres functionalized with NK-cell crosslinking antibodies and expansion cytokines;
   or a combination of any of the foregoing.

In some embodiments, the NK cells are expanded by exposure to an expansion agent comprising a combination of IL-7, IL-21, and IL-15, or functional fragments thereof, and/or fusion proteins comprising functional fragments thereof, or a combination of any of the foregoing.

In some embodiments, the NK cells are expanded by exposure to an expansion agent comprising fusion proteins comprising functional fragments of IL-7, IL-21, and IL-15.

In some embodiments, the NK cells are expanded by exposure to an expansion agent comprising 7t15-21s.

In some embodiments, the expansion agent comprises a crosslinking agent. In some embodiments, the crosslinking agent is a crosslinking antibody. In some embodiments, the crosslinking antibody is ATF1.

In some embodiments, the NK cells are expanded by exposure to an expansion agent comprising 7t15-21s and ATF1.

In some embodiments, the NK cells are expanded by exposure to an expansion agent comprising microspheres functionalized with NK-cell crosslinking antibodies and expansion cytokines.

In some embodiments, the NK cells are expanded by exposure to an expansion agent for 1 day to 40 days. In some embodiments, the NK cells are expanded by exposure to an expansion agent for 7 days to 21 days. In some embodiments, the NK cells are expanded by exposure to an expansion agent for about 14 days.

In some embodiments, the expansion agent comprises 7t15-21s and ATF1. In some embodiments, the expansion agent comprises 7t15-21s at a concentration of 0.1-300 nm and ATF1 at a concentration of 0.01-200 nm. In some embodiments, the expansion agent comprises 7t15-21s at a concentration of 0.2-200 nm and ATF1 at a concentration of 0.01-100 nm. In some embodiments, the expansion agent comprises 7t15-21s at a concentration of about 50 nm and ATF1 at a concentration of about 25 nm.

In some embodiments, the NK cells are expanded by exposure to 7t15-21s and ATF1 for about 14 days. In some embodiments, the NK cells are expanded by exposure to 7t15-21s at a concentration of about 50 nm and ATF1 at a concentration of about 25 nm for about 14 days.

In some embodiments, the NK cells are primed by exposure to a priming agent, for example chosen from a combination of cytokines, or functional fragments thereof, and/or fusion proteins comprising functional fragments thereof, or a combination of any of the foregoing.

In some embodiments, the NK cells are primed by exposure to a priming agent comprising:
one or more of IL-12, IL-23, IL-27, and IL-35;
one or more of IL-2, IL-4, IL-7, IL-9, IL-15, and IL-21; and
one or more of IL-18, IL-la, IL-1b, IL-36a, IL-36b, and IL-36g;
   or functional fragments thereof, and/or fusion proteins comprising functional fragments thereof, or a combination of any of the foregoing.

In some embodiments, the NK cells are primed by exposure to a priming agent comprising a combination of IL-12, IL-15, and IL-18.

In some embodiments, the NK cells are primed by exposure to a priming agent comprising fusion proteins comprising functional fragments of IL-12, IL-15, and IL-18. In some embodiments, the NK cells are primed by exposure to a priming agent comprising fusion protein 18t15-12s.

In some embodiments, the NK cells are primed with 18t15-12s at a concentration of 200-300 nM. In some embodiments, the NK cells are primed with 18t15-12s at a concentration of 250 nm.

In some embodiments, the NK cells are primed for 1 minute to 24 hours. In some embodiments, the NK cells are primed for 0.5 to 16 hours. In some embodiments, the NK cells are primed for 1 to 3 hours.

In some embodiments, the NK cells are cryopreserved.

In some embodiments, the NK cells are expanded first, then primed.

In some embodiments, the NK cells are expanded to greater than 10 times the starting number. In some embodiments, the NK cells are expanded to greater than 100 times the starting number. In some embodiments, the NK cells are expanded to greater than 1000 times the starting number.

In some embodiments, the NK cells are expanded and primed concurrently.

In some embodiments, the cells have a memory-like (ML) NK phenotype.

In some embodiments, the memory-like phenotype is indicated by the level of expression of cell-surface CD69, CD25, CD16, and/or NKG2A.

In some embodiments, the memory NK cells have one or more of:
a) improved cytotoxicity against cancer cells;
b) improved persistence;
c) improved anti-tumor activity; and/or
d) increased production of cytokines;
   compared to NK cells which have not been primed.

In some embodiments, the cancer cells are K562 cells.

In some embodiments, the produced cytokines are chosen from IFNg, TNFa, GM-CSF, and combinations thereof.

In some embodiments, persistence is as measured in an immunodeficient mouse for 1-14 days.

In some embodiments, the mouse is an NSG mouse.

In some embodiments, anti-tumor activity is measured as tumor growth reduction of K562 cells in an immunodeficient mouse.

In some embodiments, the NK cells are cytokine-induced memory-like (CIML) NK cells.

In some embodiments, the memory NK cells additionally comprise at least one chimeric antigen receptor (CAR), comprising:
a) at least one extracellular ligand-binding domain targeting an antigen on a target cell;
b) a hinge domain;
c) a transmembrane domain;
d) optionally, one or more co-stimulatory domains; and
e) a cytoplasmic signaling domain.

Also provided herein is a method of treating a proliferative malignancy, said method comprising administration of the memory NK cells according to the embodiments above, or cells as made by the method of the embodiments above, to a patient in need thereof.

In some embodiments, the cells are administered fresh to patients.

In some embodiments, the proliferative malignancy is a cancer.

In some embodiments, the cancer is hematologic.

In some embodiments, the hematologic cancer is chosen from leukemia, lymphoma, multiple myeloma, and myelodysplastic syndrome.

In some embodiments, the hematologic cancer is a B-cell lymphoma.

In some embodiments, the B-cell lymphoma is chosen from diffuse large B-cell lymphoma (DLBCL) and chronic lymphocytic leukemia (CLL)/small lymphocytic lymphoma (SLL).

In some embodiments, the hematologic cancer is a T-cell lymphoma.

In some embodiments, the T-cell lymphoma is chosen from T-cell acute lymphoblastic leukemia/lymphoma (T-ALL), peripheral T-cell lymphoma (PTCL), T-cell chronic lymphocytic leukemia (T-CLL), and Sezary syndrome.

In some embodiments, the hematologic cancer is a leukemia.

In some embodiments, the leukemia is chosen from acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) and hairy cell leukemia.

In some embodiments, the hematologic cancer is a plasma cell malignancy.

In some embodiments, the plasma cell malignancy is chosen from lymphoplasmacytic lymphoma, plasmacytoma, and multiple myeloma.

In some embodiments, the cancer is a solid tumor.

In some embodiments, solid tumor is chosen from a melanoma, a neuroblastoma, a glioma, a sarcoma, or a carcinoma.

In some embodiments, the solid tumor is a tumor of the brain, head, neck, breast, lung (e.g., non-small cell lung cancer, NSCLC), reproductive tract (e.g., ovary), upper digestive tract, pancreas, liver, renal system (e.g., kidneys), bladder, prostate or colorectum.

### Enumerated Embodiments

Also provided herein are the following embodiments:
Embodiment 1. A population of purified memory natural killer (NK) cells produced by, sequentially:
   a) expanding purified NK cells; and
   b) priming the NK cells.
Embodiment 2. A population of purified memory natural killer (NK) cells produced by concurrently priming and expanding purified NK cells.
Embodiment 3. The memory NK cells according to any of Embodiments 1 to 2, wherein the NK cells are enriched from fresh or frozen leukapheresate or donor blood.
Embodiment 4. The memory NK cells according to any of Embodiments 1 to 2, wherein the NK cells are differentiated from lymphoid progenitor cells.
Embodiment 5. The memory NK cells according to any of Embodiments 1 to 2, wherein the NK cells are purified by negative or positive selection, or combinations thereof.
Embodiment 6. The memory NK cells according to any of Embodiments 1 to 2, wherein the NK cells are primed by exposure to:
   one or more of IL-12, IL-23, IL-27, and IL-35;
   one or more of IL-2, IL-4, IL-7, IL-9, IL-15, and IL-21; and
   one or more of IL-18, IL-la, IL-1b, IL-36a, IL-36b, and IL-36g;
      or functional fragments thereof, and/or fusion proteins comprising functional fragments thereof, or a combination of any of the foregoing.
Embodiment 7. The memory NK cells according to Embodiment 6, wherein the NK cells are primed by exposure to 18t15-12s.
Embodiment 8. The memory NK cells according to any of Embodiments 1 to 7, wherein the NK cells are primed for 1 minute to 24 hours.
Embodiment 9. The memory NK cells according to Embodiment 6, wherein the NK cells are primed by exposure to IL-12, IL-15, and IL-18.
Embodiment 10. The memory NK cells according to Embodiment 9, wherein the NK cells are primed for 1 minute to 24 hours.
Embodiment 11. The memory NK cells according any of Embodiments 1 to 10, wherein the NK cells are expanded by exposure to 7t15-21s and ATF1.
Embodiment 12. The memory NK cells according to Embodiment 11, wherein the NK cells are expanded for 1-40 days.
Embodiment 13. The memory NK cells according to any of the previous Embodiments, wherein the memory NK phenotype is indicated by increases in CD69, CD25, and NKG2A expression, and maintenance of CD16 expression, compared to untreated NK cells.
Embodiment 14. The memory NK cells according to any of the previous Embodiments, wherein the memory NK cells have one or more of:
   improved cytotoxicity against cancer cells;
   improved persistence;
   improved anti-tumor activity; and/or
   increased production of cytokines;
      compared to untreated NK cells.
Embodiment 15. The memory NK cells according to Embodiment 14, wherein the cancer cells are K562 cells.
Embodiment 16. The memory NK cells according to Embodiment 14, wherein the produced cytokines are chosen from IFNg, TNFa, GM-CSF, and combinations thereof.
Embodiment 17. The memory NK cells according to Embodiment 14, wherein persistence is as measured in an immunodeficient mouse for 1-14 days.
Embodiment 18. The memory NK cells according to Embodiment 17, wherein the mouse is an NSG mouse.
Embodiment 19. The memory NK cells according to Embodiment 14, wherein anti-tumor activity is measured as tumor growth reduction of cancer cells in an immunodeficient mouse.
Embodiment 20. The memory NK cells according to any preceding Embodiment, wherein the NK cells are cytokine-induced memory-like (CIML) NK cells.
Embodiment 21. The memory NK cells according to any preceding Embodiment, additionally comprising at least one chimeric antigen receptor (CAR), comprising:
   a. at least one extracellular ligand-binding domain targeting an antigen on a target cell;
   b. a hinge domain;
   c. a transmembrane domain;
   d. optionally, one or more co-stimulatory domains; and
   e. a cytoplasmic signaling domain.
Embodiment 22. A method of making memory NK cells comprising:
   a) purifying an enriched population of NK cells;
   b) expanding the NK cells; and
   c) priming the NK cells.
Embodiment 23. A method of making memory NK cells comprising:
   a) purifying an enriched population of NK cells; and
   b) concurrently priming and expanding the NK cells.
Embodiment 24. The method according to any of Embodiments 22 to 23, wherein the NK cells are enriched from fresh or frozen leukapheresate or donor blood.
Embodiment 25. The method according to any of Embodiments 22 to 23, wherein the NK cells are differentiated from lymphoid progenitor cells.
Embodiment 26. The method according to any of Embodiments 22 to 23, wherein the NK cells are purified by negative or positive selection, or combinations thereof.
Embodiment 27. The method according to any of Embodiments 22 to 23, wherein the NK cells are primed by exposure to.
   one or more of IL-12, IL-23, IL-27, and IL-35;
   one or more of IL-2, IL-4, IL-7, IL-9, IL-15, and IL-21; and
   one or more of IL-18, IL-la, IL-1b, IL-36a, IL-36b, and IL-36g;
      or functional fragments thereof, and/or fusion proteins comprising functional fragments thereof, or a combination of any of the foregoing.
Embodiment 28. The method according to Embodiment 27, wherein the NK cells are primed by exposure to 18t15-12s.
Embodiment 29. The method according to Embodiment 28, wherein the NK cells are primed for 1 minute-24 hours.
Embodiment 30. The method according to Embodiment 27, wherein the NK cells are primed by exposure to IL-12, IL-15, and IL-18.
Embodiment 31. The method according to Embodiment 28, wherein the NK cells are primed for 2-40 days.
Embodiment 32. The method according any of Embodiments 22 to 23, wherein the NK cells are expanded by exposure to 7t15-21s and ATF1.
Embodiment 33. The method according any of Embodiments 22 to 23, wherein the NK cells are expanded for 1-40 days.
Embodiment 34. The method according to any of the previous Embodiments, wherein the memory NK phenotype is indicated by increases in CD69, CD25, and NKG2A expression, and maintenance of CD16 expression, compared to untreated NK cells.
Embodiment 35. The method according any of Embodiments 22 to 34, wherein the memory NK cells have one or more of:
   improved cytotoxicity against cancer cells;
   improved persistence;
   improved anti-tumor activity; and/or
   increased production of cytokines;
      compared to untreated NK cells.
Embodiment 36. The method according to Embodiment 35, wherein the cancer cells are K562 cells.
Embodiment 37. The method according to Embodiment 35, wherein the produced cytokines are chosen from IFNg, TNFa, GM-CSF, and combinations thereof.
Embodiment 38. The method according to Embodiment 35, wherein persistence is as measured in an immunodeficient mouse for 1-14 days.
Embodiment 39. The method according to Embodiment 38, wherein the mouse is an NSG mouse.
Embodiment 40. The method according to Embodiment 35, wherein the improved anti-tumor activity is tumor growth reduction of cancer cells in an immunodeficient mouse.
Embodiment 41. The method according to any of the previous Embodiments, wherein the cells are cytokine-induced ML (CIML) NK cells.
Embodiment 42. A method of treating a proliferative malignancy, the method comprising administration of the memory NK cells according to any of Embodiments 1-21, or memory NK cells as made by the method of any of Embodiments 22-41, to a patient in need thereof.
Embodiment 43. The method of Embodiment 42, wherein the cells are administered fresh to patients.
Embodiment 44. The method of Embodiment 42, wherein the proliferative malignancy is a cancer.
Embodiment 45. The method of Embodiment 44, wherein the cancer is hematologic.
Embodiment 46. The method of Embodiment 44, wherein the hematologic cancer is chosen from leukemia, lymphoma, multiple myeloma, and myelodysplastic syndrome.
Embodiment 47. The method of Embodiment 46, wherein the hematologic cancer is a B-cell lymphoma.
Embodiment 48. The method of Embodiment 47, wherein the B-cell lymphoma is chosen from diffuse large B-cell lymphoma (DLBCL) and chronic lymphocytic leukemia (CLL)/small lymphocytic lymphoma (SLL).
Embodiment 49. The method of Embodiment 46, wherein the hematologic cancer is a T-cell lymphoma.
Embodiment 50. The method of Embodiment 49, wherein the T-cell lymphoma is chosen from T-cell acute lymphoblastic leukemia/lymphoma (T-ALL), peripheral T-cell lymphoma (PTCL), T-cell chronic lymphocytic leukemia (T-CLL), and Sezary syndrome.
Embodiment 51. The method of Embodiment 46, wherein the hematologic cancer is a leukemia.
Embodiment 52. The method of Embodiment 51, wherein the leukemias is chosen from acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) and hairy cell leukemia.
Embodiment 53. The method of Embodiment 46, wherein the hematologic cancer is a plasma cell malignancy.
Embodiment 54. The method of Embodiment 53, wherein the plasma cell malignancy is chosen from lymphoplasmacytic lymphoma, plasmacytoma, and multiple myeloma.
Embodiment 55. The method of Embodiment 44, wherein the cancer is a solid tumor.
Embodiment 56. The method of Embodiment 55, wherein the solid tumor is chosen from a melanoma, a neuroblastoma, a glioma, a sarcoma, or a carcinoma
Embodiment 57. The method of Embodiment 55, wherein the solid tumor is a tumor of the brain, head, neck, breast, lung (e.g., non-small cell lung cancer, NSCLC), reproductive tract (e.g., ovary), upper digestive tract, pancreas, liver, renal system (e.g., kidneys), bladder, prostate or colorectum.

### Methods of Expansion and Priming of Immune Effector Cells

Expansion of the NK cells in vitro may be performed in an enrichment process that uses an expanding agent comprising cytokines, or, preferably, expansion fusion proteins comprising functional fragments of cytokines, and multichain complexes thereof. For example, the expanding agent may comprise one or more of IL-2, IL-4, IL-7, IL-9, IL-15, and IL-21, or a combination thereof, for example a cocktail of IL-7, IL-21, and IL-15, in an amount sufficient to produce a desired quantity or fold expansion of NK cells. Such cytokines may be obtained commercially or made by methods known in the art. Or, for example, the expanding agent may comprise one or more expansion fusion proteins, e.g., may be chosen from amongst multi-chain fusion protein complexes disclosed in WO2020047299, WO202047473, or WO 2020257639, for example 7t15-21s, in an amount sufficient to expand NK cells. The sequences of 7t15-21s are disclosed in Table 1.

TABLE 1
Sequences of Exemplary Expansion Fusion Protein Chains

| | WO'299 | | |
|---|---|---|---|
| | SEQ ID | | |
| Element | NO. | SEQ ID NO. | Sequence |
| Mature 7t15 | 104 | SEQ ID | DCDIEGKDGKQYESVLMVSIDQLLDSM |
| | | NO: 49 | KEIGSNCLNNEFNFFKRHICDANKEGMF |
| | | | LFRAARKLRQFLKMNSTGDFDLHLLKV |
| | | | SEGTTILLNCTGQVKGRKPAALGEAQPT |
| | | | KSLEENKSLKEQKKLNDLCFLKRLLQEI |
| | | | KTCWNKILMGTKEHSGTTNTVAAYNL |
| | | | TWKSTNFKTILEWEPKPVNQVYTVQIST |
| | | | KSGDWKSKCFYTTDTECDLTDEIVKDV |
| | | | KQTYLARVFSYPAGNVESTGSAGEPLY |
| | | | ENSPEFTPYLETNLGQPTIQSFEQVGTK |
| | | | VNVTVEDERTLVRRNNTFLSLRDVFGK |
| | | | DLIYTLYYWKSSSSGKKTAKTNTNEFLI |
| | | | DVDKGENYCFSVQAVIPSRTVNRKSTD |
| | | | SPVECMGQEKGEFRENWVNVISDLKKI |
| | | | EDLIQSMHIDATLYTESDVHPSCKVTA |
| | | | MKCFLLELQVISLESGDASIHDTVENLII |
| | | | LANNSLSSNGNVTESGCKECEELEEKNI |
| | | | KEFLQSFVHIVQMFINTS |
| Mature 21s | 108 | SEQ ID | QGQDRHMIRMRQLIDIVDQLKNYVNDL |
| | | NO: 50 | VPEFLPAPEDVETNCEWSAFSCFQKAQL |
| | | | KSANTGNNERIINVSIKKLKRKPPSTNA |
| | | | GRRQKHRLTCPSCDSYEKKPPKEFLERF |
| | | | KSLLQKMIHQHLSSRTHGSEDSITCPPP |
| | | | MSVEHADIWVKSYSLYSRERYICNSGF |
| | | | KRKAGTSSLTECVLNKATNVAHWTTPS |
| | | | LKCIR |

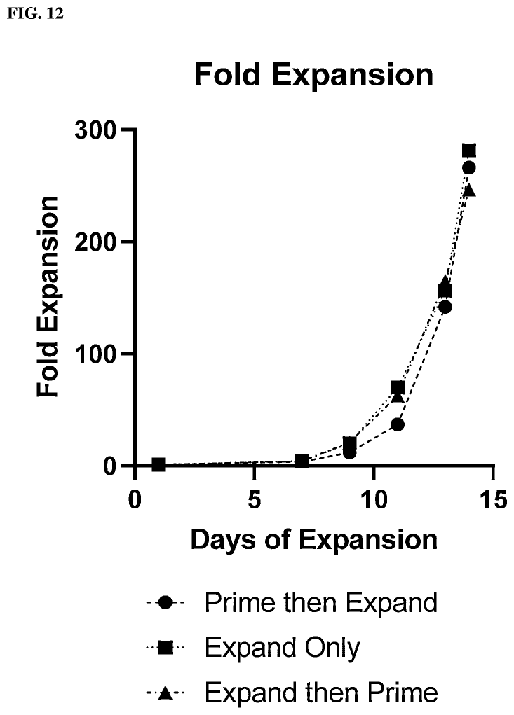

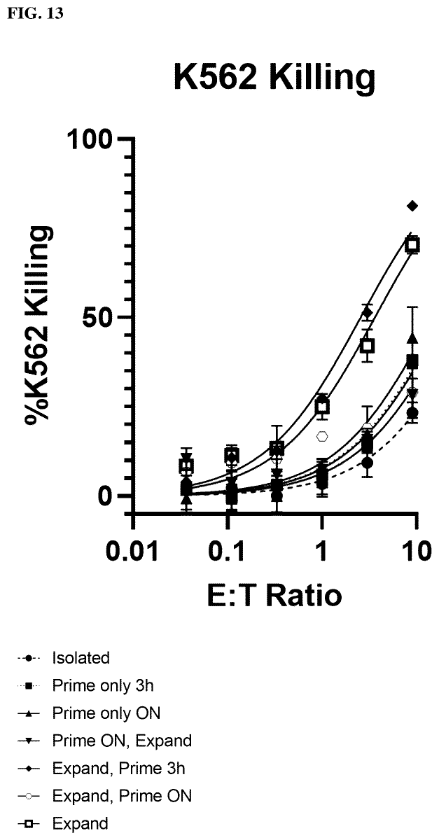

Expansion is additionally facilitated by use of a cross-linking agent, such as an antibody targeting a linking domain of the fusion proteins disclosed above, for example an anti-tissue-factor antibody. Examples of anti-tissue factor antibodies are known in the art. WO202047473 and WO2020257639 disclose the a-TF Ab to be used. See also, U.S. Pat. No. 8,007,795 and WO2003037911, in particular IgG1 humanized antibodies incorporating the CDRs of the H36 hybridoma and humanized framework regions LC-08 (FIG. 12) and HC-09 (FIG. 13). Table X below discloses the sequences of the a-TF Ab believed to be used in WO'473 and WO'639, disclosed in US'795 and WO'911, obtained from HCW Biologics, and used in the experiments below unless otherwise stated, referred to herein as ATF1. ATF1 HCDR2 is one of the two sequences below. Accordingly, an expansion agent as disclosed herein may comprise a combination of one or more cytokines or an EFP as disclosed above, together with a crosslinking agent such as ATF1, the sequence(s) of which are disclosed in Table 2.

TABLE 2
Sequences of Exemplary Anti-TF Antibodies (“ATF1”)

| a-TF | US'795 | | |
|---|---|---|---|
| Antibody | SEQ | | |
| Element | ID NO. | SEQ ID NO. | Sequence |
| LC-CDR1 | 116 | SEQ ID NO: 51 | LASQTIDTWLA |
| LC-CDR2 | 6 | SEQ ID NO: 52 | AATNLAD |
| LC-CDR3 | 7 | SEQ ID NO: 53 | QQVYSSPFT |
| LC-FR1 | 109 | SEQ ID NO: 54 | DIQMTQSPASLSASVGDRVTITC |
| LC-FR2 | 108 | SEQ ID NO: 55 | WYLQKPGKSPQLLIY |
| LC-FR3 | 112 | SEQ ID NO: 56 | GVPSRFSGSGSGTDFSFTISSLQPEDFATYY |
| | | | C |
| LC-FR4 | 110 | SEQ ID NO: 57 | FGQGTKLEIK |
| VL | n/a | SEQ ID NO: 58 | DIQMTQSPASLSASVGDRVTITCLASQTID |
| | | | TWLAWYLQKPGKSPQLLIYAATNLADGV |
| | | | PSRFSGSGSGTDFSFTISSLQPEDFATYYCQ |
| | | | QVYSSPFT GQGTKLEIK |
| HC- | 134 | SEQ ID NO: 59 | DYNVY |
| CDR1 | | | |
| HC- | 9 | SEQ ID NO: 60 | YIDPYNGITIYDQNFKG |
| CDR2 (a) | | | |
| HC- | 101 | SEQ ID NO: 61 | YIDPYNGITIYDQNLKG |
| CDR2 (b) | | | |
| HC- | 10 | SEQ ID NO: 62 | DVTTALDF |
| CDR3 | | | |
| HC-FR1 | 129 | SEQ ID NO: 63 | QIQLVQSGGEVKKPGASVRVSCKASGYSF |
| | | | T |
| HC-FR2 | 123 | SEQ ID NO: 64 | WVRQSPGKGLEWIG |
| HC-FR3 | 126 | SEQ ID NO: 65 | KATLTVDKSTSTAYMELSSLRSEDTAVYF |
| | | | CAR |
| HC-FR4 | 122 | SEQ ID NO: 66 | WGQGTTVTVSS |
| VH-1 | n/a | SEQ ID NO: 67 | QIQLVQSGGEVKKPGASVRVSCKASGYSF |
| | | | TDYNVYWVRQSPGKGLEWIGYIDPYNGIT |
| | | | IYDQNFKGKATLTVDKSTSTAYMELSSLR |
| | | | SEDTAVYFCARDVTTALDFWGQGTTVTV |
| | | | SS |
| VH-2 | n/a | SEQ ID NO: 68 | QIQLVQSGGEVKKPGASVRVSCKASGYSF |
| | | | TDYNVYWVRQSPGKGLEWIGYIDPYNGIT |
| | | | IYDQNLKGKATLTVDKSTSTAYMELSSLR |
| | | | SEDTAVYFCARDVTTALDFWGQGTTVTV |
| | | | SS |
| IgG1 LC | 97 | SEQ ID NO: 69 | RTVAAPSVFIFPPSDEQLKSGTASVVCLLN |
| constant | | | NFYPREAKVQWKVDNALQSGNSQESVTE |
| region | | | QDSKDSTYSLSSTLTLSKADYEKHKVYAC |
| | | | EVTHQGLSSPVTKSFNRGEC |
| IgG1 HC | 98 | SEQ ID NO: 70 | RTVAAPSVFIFPPSDEQLKSGTASVVCLLN |
| constant | | | NFYPREAKVQWKVDNALQEFASTKGPSV |
| region | | | FPLAPSSKSTSGGTAALGCLVKDYFPEPVT |
| | | | VSWNSGALTSGVHTFPAVLQSSGLYSLSS |
| | | | VVTVPSSSLGTQTYICNVNHKPSNTKVDK |
| | | | KVEPKSCDKTHTCPPCPAPELLGGPSVFLF |
| | | | PPKPKDTLMISRTPEVTCVVVDVSHEDPE |
| | | | VKFNWYVDGVEVHNAKTKPREEQYNSTY |
| | | | RVVSVLTVLHQDWLNGKEYKCKVSNKAL |
| | | | PAPIEKTISKAKGQPREPQVYTLPPSRDELT |
| | | | KNQVSLTCLVKG+C7FYPSDIAVEWESNG |
| | | | QPENNYKTTPPVLDSDGSFFLYSKLTVDK |
| | | | SRWQQGNVFSCSVMHEALHNHYTQKSLS |
| | | | LSPGK |

Alternative methods of cross-linking are known in the art, and include functionalized microparticles (beads), feeder cells and plasma membrane particles. Feeder-free systems are often preferred. For example, R&D Systems Cloudz human NK cell expansion kits, employing dissolvable sodium alginate microspheres that are functionalized with anti-CD2 and anti-NKp46 antibodies, may be used with expansion cytokines (or fragments thereof, or fusion proteins comprising) and combinations thereof as disclosed herein, along with a release buffer after expansion for quickly dissolving microparticles, facilitating cell harvesting.

Priming to obtain the memory like character is performed with a priming agent comprising a combination of stimulatory cytokines, such as one or more of IL-12, IL-23, IL-27, and IL-35; one or more of IL-2, IL-4, IL-7, IL-9, IL-15, and IL-21; and one or more of IL-18, IL-la, IL-1b, IL-36a, IL-36b, and IL-36g. Alternatively, the priming agent may comprise priming fusion proteins comprising functional fragments of cytokines, and multichain complexes thereof. For example, the fusion proteins may be chosen from amongst multi-chain fusion protein complexes disclosed in WO2020047299, WO202047473, or WO 2020257639, for example 18t15-12s (HCW-9201), the sequences of which are disclosed in Table 3.

TABLE 3
Sequences of Exemplary Priming Fusion Protein Chains

| | WO'299 | | |
|---|---|---|---|
| | SEQ ID | | |
| Element | NO. | SEQ ID NO. | Sequence |
| Mature 18t15 | 70 | SEQ ID | YFGKLESKLSVIRNLNMTDSDCRDNAPRT |
| | | NO: 71 | IFIISMYKDSQPRGMAVTISVKCEKISTLSC |
| | | | ENKIISFKEMNPPDNIKDTKSDIIFFQRSVP |
| | | | GHDNKMQFESSSYEGYFLACEKERDLFK |
| | | | LILKKEDELGDRSIMFTVQNEDSGTTNTV |
| | | | AAYNLTWKSTNFKTILEWEPKPVNQVYT |
| | | | VQISTKSGDWKSKCFYTTDTECDLTDEIV |
| | | | KDVKQTYLARVFSYPAGNVESTGSAGEP |
| | | | LYENSPEFTPYLETNLGQPTIQSFEQVGTK |
| | | | VNVTVEDERTLVRRNNTFLSLRDVFGKD |
| | | | LIYTLYYWKSSSSGKKTAKTNTNEFLIDV |
| | | | DKGENYCFSVQAVIPSRTVNRKSTDSPVE |
| | | | CMGQEKGEFRENWVNVISDLKKIEDLIQS |
| | | | MHIDATLYTESDVHPSCKVTAMKCFLLE |
| | | | LQVISLESGDASIHDTVENLIILANNSLSSN |
| | | | GNVTESGCKECEELEEKNIKEFLQSFVHIV |
| | | | QMFINTS |
| Mature 12s | 74 | SEQ ID | IWELKKDVYVVELDWYPDAPGEMVVLT |
| | | NO: 72 | CDTPEEDGITWTLDQSSEVLGSGKTLTIQ |
| | | | VKEFGDAGQYTCHKGGEVLSHSLLLLHK |
| | | | KEDGIWSTDILKDQKEPKNKTFLRCEAKN |
| | | | YSGRFTCWWLTTISTDLTFSVKSSRGSSD |
| | | | PQGVTCGAATLSAERVRGDNKEYEYSVE |
| | | | CQEDSACPAAEESLPIEVMVDAVHKLKY |
| | | | ENYTSSFFIRDIIKPDPPKNLQLKPLKNSR |
| | | | QVEVSWEYPDTWSTPHSYFSLTFCVQVQ |
| | | | GKSKREKKDRVFTDKTSATVICRKNASIS |
| | | | VRAQDRYYSSSWSEWASVPCSGGGGSG |
| | | | GGGSGGGGSRNLPVATPDPGMFPCLHHS |
| | | | QNLLRAVSNMLQKARQTLEFYPCTSEEID |
| | | | HEDITKDKTSTVEACLPLELTKNESCLNS |
| | | | RETSFITNGSCLASRKTSFMMALCLSSIYE |
| | | | DLKMYQVEFKTMNAKLLMDPKRQIFLD |
| | | | QNMLAVIDELMQALNFNSETVPQKSSLE |
| | | | EPDFYKTKIKLCILLHAFRIRAVTIDRVMS |
| | | | YLNASITCPPPMSVEHADIWVKSYSLYSR |
| | | | ERYICNSGFKRKAGTSSLTECVLNKATNV |
| | | | AHWTTPSLKCIR |

### Chimeric Antigen Receptors (CARs) and CAR-Bearing Immune Effector Cells

Also provided herein are chimeric antigen receptors (CARs) comprising polypeptides as disclosed herein, and immune effector cells expressing them. A CAR is a recombinant fusion protein typically comprising: 1) an extracellular ligand-binding domain, i.e., an antigen-recognition domain, 2) a hinge domain, 3) a transmembrane domain, and 4) a cytoplasmic signaling domain, 5) and optionally, a co-stimulatory domain.

Methods for CAR design, delivery and expression, and the manufacturing of clinical-grade CAR-expressing cell populations are known in the art. CAR designs are generally tailored to each cell type.

The extracellular ligand-binding domain of a chimeric antigen receptor recognizes and specifically binds an antigen, typically a surface-expressed antigen of a malignant cell. The extracellular ligand-binding domain specifically binds an antigen when, for example, it binds the antigen with an affinity constant or affinity of interaction (K_{D}) between about 0.1 pM to about 10 μM, or about 0.1 pM to about 1 μM, or about 0.1 pM to about 100 nM. Methods for determining the affinity of interaction are known in the art. An extracellular ligand-binding domain can also be said to specifically bind a first polymorphic variant of an antigen when it binds it selectively over a second polymorphic variant of the same antigen.

An extracellular ligand-binding domain suitable for use in a CAR may be any antigen-binding polypeptide, a wide variety of which are known in the art. In some instances, the extracellular ligand-binding domain is a single chain Fv (scFv). Other antibody-based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use. In some embodiments, the extracellular ligand-binding domain is constructed from a natural binding partner, or a functional fragment thereof, to a target antigen. For example, CARs in general may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma.

The targeted antigen to which the CAR binds via its extracellular ligand-binding domain may be an antigen that is expressed on a malignant myeloid (AML) cell, T cell or other cell. Antigens expressed on malignant myeloid (AML) cells include CD33, FLT3, CD123, and CLL-1. Antigens expressed on T cells include CD2, CD3, CD4, CD5, CD7, TCRα (TRAC), and TCRβ. Antigens expressed on malignant plasma cells include BCMA, CS1, CD38, CD79A, CD79B, CD138, and CD19. Antigens expressed on malignant B cells include CD19, CD20, CD21, CD22, CD23, CD24, CD25, CD27, CD38, and CD45.

Typically, the extracellular ligand-binding domain is linked to the intracellular domain of the chimeric antigen receptor by a transmembrane (TM) domain. A peptide hinge connects the extracellular ligand-binding domain to the transmembrane domain. A transmembrane domain traverses the cell membrane, anchors the CAR to the T cell surface, and connects the extracellular ligand-binding to the cytoplasmic signaling domain, thus impacting expression of the CAR on the T cell surface.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively, the transmembrane domain can be synthetic and comprise predominantly hydrophobic amino acid residues (e.g., leucine and valine). In some cases, a triplet of phenylalanine, tryptophan and valine will be found at each end of a synthetic transmembrane domain. In some embodiments, the transmembrane domain is derived from the T-cell surface glycoprotein CD8 alpha chain isoform 1 precursor (NP_001139345.1) or CD28. A short oligo-or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain, or can be different transmembrane domains.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. In some embodiments, the TM adapter may be chosen from FceR 1γ (ITAMx1), CD3ζ (ITAMx3), DAP12 (ITAMx1), or DAP10 (YxxM/YINM), NKG2D, FcγRIIIa, NKp44, NKp30, NKp46, actKIR, NKG2C, CD8α, and IL15Rb.

The CAR can further comprise a hinge region between extracellular ligand-binding domain and the transmembrane domain. The term “hinge region” (equivalently, “hinge” or “spacer”) generally means any oligo-or polypeptide that functions to link the transmembrane domain to the extracellular ligand-binding domain. In particular, hinge region is used to provide more flexibility and accessibility for the extracellular ligand-binding domain, and can confer stability for efficient CAR expression and activity. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), OX-40 (CD134), CD3ζ, the T cell receptor α or β chain, CD45, CD4, CD5, CD8, CD8a, CD9, CD16, CD22, CD33, CD37, CD64, CD80, CD86, ICOS, CD154 or from all or parts of an antibody constant region. In some embodiments, for example, the hinge sequence is derived from a CD8a molecule or a CD28 molecule. Alternatively, the hinge region may be a synthetic sequence that corresponds to a naturally-occurring hinge sequence or the hinge region may be an entirely synthetic hinge sequence. In one embodiment, the hinge domain comprises a part of human CD8α (SEQ ID NO:2), FcγRIIIα receptor, or IgGl, and have at least 80%, 90%, 95%, 97%, or 99% sequence identity thereto.

After antigen recognition, the cytoplasmic signaling domain transmits a signal to the immune effector cell, activating at least one of the normal effector functions of the immune effector cell. Effector function of an NK cell, for example, may be cytolytic activity or helper activity including the secretion of cytokines. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function

Cytoplasmic signaling sequences that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

First-generation CARs typically have the cytoplasmic signaling domain from the CD3 chain, which is the primary transmitter of signals from endogenous TCRs. Second-generation CARs add cytoplasmic signaling domains from various co-stimulatory protein receptors (e.g., CD28, 4-1BB, ICOS) to the cytoplasmic signaling domain of the CAR to provide additional signals to the cell.

A “costimulatory domain” is derived from the intracellular signaling domains of costimulatory proteins that enhance cytokine production, proliferation, cytotoxicity, and/or persistence in vivo. Preclinical studies have indicated that the second generation of CAR designs improves antitumor activity. More recent, third-generation, and later generation, CARs combine multiple costimulatory domains to further augment potency. Cells grafted with these CARs have demonstrated improved expansion, activation, persistence, and tumor-eradicating efficiency independent of costimulatory receptor/ligand interaction.

For example, the cytoplasmic signaling domain of the CAR can be designed to comprise the signaling domain (e.g., CD3ζ) by itself or combined with any other desired cytoplasmic domain(s) useful in the context of the CAR. For example, the cytoplasmic domain of the CAR can comprise a signaling domain (e.g., CD3ζ) chain portion and a costimulatory signaling region. The co-stimulatory signaling region refers to a portion of the CAR comprising the intracellular domain of a co-stimulatory molecule. Examples of such molecules include CD27, CD28, 4-1BB (CD137), OX40, CD30, CD40, ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

In some embodiments, the cytoplasmic signaling domain is a CD3 zeta (CD3ζ) signaling domain. In some embodiments, the co-stimulatory domain comprises the cytoplasmic domain of CD28, 4-1BB, or a combination thereof. In some cases, the co-stimulatory signaling region contains 1, 2, 3, or 4 cytoplasmic domains of one or more intracellular signaling and/or co-stimulatory molecules.

The co-stimulatory signaling domain(s) may contain one or more mutations in the cytoplasmic domains of CD28 and/or 4-1BB that enhance signaling. In some embodiments, the disclosed CARs comprise a co-stimulatory signaling region comprising a mutated form of the cytoplasmic domain of CD28 with altered phosphorylation at Y206 and/or Y218. In some embodiments, the disclosed CAR comprises an attenuating mutation at Y206, which will reduce the activity of the CAR. In some embodiments, the disclosed CAR comprises an attenuating mutation at Y218, which will reduce expression of the CAR. Any amino acid residue, such as alanine or phenylalanine, can be substituted for the tyrosine to achieve attenuation. In some embodiments, the tyrosine at Y206 and/or Y218 is substituted with a phosphomimetic residue. In some embodiments, the disclosed CAR substitution of Y206 with a phosphomimetic residue, which will increase the activity of the CAR. In some embodiments, the disclosed CAR comprises substitution of Y218 with a phosphomimetic residue, which will increase expression of the CAR. For example, the phosphomimetic residue can be phosphotyrosine. In some embodiments, a CAR may contain a combination of phosphomimetic amino acids and substitution(s) with non-phosphorylatable amino acids in different residues of the same CAR. For instance, a CAR may contain an alanine or phenylalanine substitution in Y209 and/or Y191 plus a phosphomimetic substitution in Y206 and/or Y218.

In some embodiments, the disclosed CARs comprise one or more 4-1BB domains with mutations that enhance binding to specific TRAF proteins, such as TRAF1,TRAF2, TRAF3, TRAF4, TRAF5, TRAF6, or any combination thereof. In some cases, the 41BB mutation enhances TRAF1- and/or TRAF2-dependent proliferation and survival of the T-cell, e.g., through NF-κB. In some cases, the 4-1BB mutation enhances TRAF3-dependent antitumor efficacy, e.g., through IRF7/INFβ. Therefore, the disclosed CARs can comprise cytoplasmic domain(s) of 4-1BB having at least one mutation in these underligned sequences that enhance TRAF-binding and/or enhance NFκB signaling.

Also as disclosed herein, TRAF proteins can in some cases enhance CAR T cell function independent of NFκB and 4-1BB. For example, TRAF proteins can in some cases enhance CD28 co-stimulation in T cells. Therefore, also disclosed herein are immune effector cells co-expressing CARs with one or more TRAF proteins, such as TRAF1, TRAF2, TRAF3, TRAF4, TRAF5, TRAF6, or any combination thereof. In some cases, the CAR is any CAR that targets a tumor antigen. For example, first-generation CARs typically had the intracellular domain from the CD3 chain, while second-generation CARs added intracellular signaling domains from various costimulatory protein receptors (e.g., CD28, 4-1BB, ICOS) to the cytoplasmic signaling domain of the CAR to provide additional signals to the T cell. In some cases, the CAR is the disclosed CAR with enhanced 4-1BB activation.

Variations on CAR components may be advantageous, depending upon the type of cell in which the CAR is expressed.

For example, in NK cells, in some embodiments, the transmembrane domain can be a sequence associated with NKG2D, FcγRIIIa, NKp44, NKp30, NKp46, actKIR, NKG2C, or CD8α. In certain embodiments, the NK cell is a ML-NK or CIML-NK cell and the TM domain is CD8 α. Certain TM domains that do not work well in NK cells generally may work in a subset; CD8α, for example, works in ML-NKs but not NK cells generally.

Similarly, in NK cells, in some embodiments, the intracellular signaling domain(s) can be any co-activating receptor(s) capable of functioning in an NK cell, such as, for example, CD28, CD137/41BB (TRAF, NFκB), CD134/OX40, CD278/ICOS, DNAM-1 (Y-motif), NKp80 (Y-motif), 2B4 (SLAMF)::ITSM, CRACC (CS1/SLAMF7):: ITSM, CD2 (Y-motifs, MAPK/Erk), CD27 (TRAF, NFκB), or integrins (e.g., multiple integrins).

Similarly, in NK cells, in some embodiments, an intracellular signaling domain can be a cytokine receptor capable of functioning in an NK cell. For example, a cytokine receptor can be a cytokine receptor associated with persistence, survival, or metabolism, such as IL-2/15Rbyc::Jak1/3, STAT3/5, PI3K/mTOR, MAPK/ERK. As another example, a cytokine receptor can be a cytokine receptor associated with activation, such as IL-18R::NFκB. As another example, a cytokine receptor can be a cytokine receptor associated with IFN-γ production, such as IL-12R::STAT4. As another example, a cytokine receptor can be a cytokine receptor associated with cytotoxicity or persistence, such as IL-21R::Jak3/Tyk2, or STAT3. As another example, an intracellular signaling domain can be a TM adapter, such as FceR1γ (ITAMx1), CD3ζ (ITAMx3), DAP12 (ITAMx1), or DAP10 (YxxM/YINM). As another example, CAR intracellular signaling domains (also known as endodomains) can be derived from costimulatory molecules from the CD28 family (such as CD28 and ICOS) or the tumor necrosis factor receptor (TNFR) family of genes (such as 4-1BB, OX40, or CD27). The TNFR family members signal through recruitment of TRAF proteins and are associated with cellular activation, differentiation and survival. Certain signaling domains that may not work well in all NK cells generally may work in a subset; CD28 or 4-1BB, for example, work in ML-NKs.

Any domain of a CAR may also comprise a heterodimerizing domain for the aim of splitting key signaling and antigen recognition modules of the CAR.

A CAR may be designed to comprise any portion or part of the above-mentioned domains as described herein in any combination resulting in a functional CAR.

### Methods of Making CARs and CAR-Bearing Cells

The chimeric antigen receptor (CAR) construct, which encodes the chimeric receptor can be prepared in conventional ways. Since, for the most part, natural sequences are employed, the natural genes are isolated and manipulated, as appropriate (e.g., when employing a Type II receptor, the immune signaling receptor component may have to be inverted), so as to allow for the proper joining of the various components. Thus, the nucleic acid sequences encoding for the N-terminal and C-terminal proteins of the chimeric receptor can be isolated by employing the polymerase chain reaction (PCR), using appropriate primers which result in deletion of the undesired portions of the gene. Alternatively, restriction digests of cloned genes can be used to generate the chimeric construct. In either case, the sequences can be selected to provide for restriction sites which are blunt-ended, or have complementary overlaps.

The various manipulations for preparing the chimeric construct can be carried out in vitro and in particular embodiments the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

A chimeric construct can be introduced into immune effector cells as naked DNA or in a suitable vector. Methods of stably transfecting immune effector cells by electroporation using naked DNA are known in the art. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cell, e.g., T cells. Suitable vectors are non-replicating in the immune effector cells of the subject. A large number of vectors are known which are based on viruses, where the copy number of the virus maintained in the cell is low enough to maintain the viability of the cell. Illustrative vectors include the pFB-neo vectors (STRATAGENE™) as well as vectors based on HIV, SV40, EBV, HSV or BPV. Once it is established that the transfected or transduced immune effector cell is capable of expressing the chimeric receptor as a surface membrane protein with the desired regulation and at a desired level, it can be determined whether the chimeric receptor is functional in the host cell to provide for the desired signal induction (e.g., production of Rantes, Mip1-alpha, GM-CSF upon stimulation with the appropriate ligand).

Engineered CARs may be introduced into CAR-bearing immune effector cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3,Csf4, and Cu1966, etc.). Zinc finger nucleases (ZFNs) and transcription activator-like effector nucleases (TALENs) may also be used. See, e.g., Shearer R F and Saunders D N, “Experimental design for stable genetic manipulation in mammalian cell lines: lentivirus and alternatives,” Genes Cells 2015 January; 20 (1):1-10. Base-editing CRISPR systems comprising a Cas-CRISPR protein fused to a base-editing protein such as a deaminase may also be used (e.g., those from Beam Therapeutics).

Amino acid sequences for selected components which may be used to construct a CAR are disclosed below in Table 4 and Table 5.

TABLE 4
Amino acid sequences of selected CAR components.

| | SEQ ID | |
|---|---|---|
| Functional domains | NO: | Amino acid sequence |
| CD8α signal/leader | SEQ ID | MALPVTALLLPLALLLHAARP |
| peptide (variant 1) | NO: 1 | |
| CD8α signal/leader | SEQ ID | MALPVTALLLPLALLLHAA |
| peptide (variant 2) | NO: 2 | |
| CD8α signal/leader | SEQ ID | MALPVTALLLP |
| peptide (variant 3) | NO: 3 | |
| CD8α signal/leader | SEQ ID | PVTALLLPLALL |
| peptide (variant 4) | NO: 4 | |
| CD8α signal/leader | SEQ ID | LLLPLALLLHAARP |
| peptide (variant 5) | NO: 5 | |
| CD8α hinge | SEQ ID | TTTPAPRPPTPAPTIASQPLSLRPEACRPAAGGAVHTR |
| | NO: 6 | GLDFACD |
| CD28 Transmembrane | SEQ ID | FWVLVVVGGVLACYSLLVTVAFIIFWV |
| (Tₘ) domain | NO: 7 | |
| Surface glycoprotein | SEQ ID | MALPVTALLLPLALLLHAARPSQFRVSPLDRTWNLGE |
| CD8 alpha chain | NO: 8 | TVELKCQVLLSNPTSGCSWLFQPRGAAASPTFLLYLS |
| isoform 1 precursor | | QNKPKAAEGLDTQRFSGKRLGDTFVLTLSDFRRENE |
| (NP_001139345.1) | | GYYFCSALSNSIMYFSHFVPVFLPAKPTTTPAPRPPTP |
| | | APTIASQPLSLRPEACRPAAGGAVHTRGLDFACDIYI |
| | | WAPLAGTCGVLLLSLVITLYCNHRNRRRVCKCPRPV |
| | | VKSGDKPSLSARYV |
| 4-1BB costimulatory | SEQ ID | KRGRKKLLYIFKQPFMRPVQTTQEEDGCSCRFPEEEE |
| domain | NO: 9 | GGCEL |
| CD28 costimulatory | SEQ ID | RSKRSRLLHSDYMNMTPRRPGPTRKHYQPYAPPRDF |
| domain | NO: 10 | AAYRS |
| CD2 costimulatory | SEQ ID | KRKKQRSRRNDEELETRAHRVATEERGRKPHQIPAST |
| domain | NO: 11 | PQNPATSQHPPPPPGHRSQAPSHRPPPPGHRVQHQPQ |
| | | KRPPAPSGTQVHQQKGPPLPRPRVQPKPPHGAAENSL |
| | | SPSSN |
| CD4 costimulatory | SEQ ID | CVRCRHRRRQAERMSQIKRLLSEKKTCQCPHRFQKT |
| domain | NO: 12 | CSPI |
| CD8a costimulatory | SEQ ID | LYCNHRN RRRVCKCPRP VVKSGDKPSL SARYV |
| domain | NO: 13 | |
| CD8b costimulatory | SEQ ID | HLCCRRRRA RLRFMKQFYK |
| domain | NO: 14 | |
| LAT costimulatory | SEQ ID | HCHRLPGSYDSTSSDSLYPRGIQFKRPHTVAPWPPAY |
| domain | NO: 15 | PPVTSYPPLSQPDLLPIPRSPQPLGGSHRTPSSRRDSDG |
| | | ANSVASYENEGASGIRGAQAGWGVWGPSWTRLTPV |
| | | SLPPEPACEDADEDEDDYHNPGYLVVLPDSTPATSTA |
| | | APSAPALSTPGIRDSAFSMESIDDYVNVPESGESAEAS |
| | | LDGSREYVNVSQELHPGAAKTEPAALSSQEAEEVEEE |
| | | GAPDYENLQELN |
| CD3 zeta (ζ) | SEQ ID | RVKFSRSADAPAYKQGQNQLYNELNLGRREEYDVLD |
| | NO: 16 | KRRGRDPEMGGKPRRKNPQEGLYNELQKDKMAEAY |
| | | SEIGMKGERRRGKGHDGLYQGLSTATKDTYDALHM |
| | | QALPPR |
| P2A peptide | SEQ ID | GSGATNFSLLKQAGDVEENPGP |
| | NO: 17 | |
| (GGGGS)₄ linker | SEQ ID | GGGGSGGGGSGGGGSGGGGS |
| | NO: 18 | |
| (GGGGS)₃ linker | SEQ ID | GGGGSGGGGSGGGGS |
| | NO: 19 | |
| (GGGGS)₂ linker | SEQ ID | GGGGGGGGS |
| | NO: 20 | |
| (GGGGS)₁ linker | SEQ ID | GGGGS |
| | NO: 21 | |
| hCD34 | SEQ ID | MPRGWTALCLLSLLPSGFMSLDNNGTATPELPTQGTF |
| | NO: 22 | SNVSTNVSYQETTTPSTLGSTSLHPVSQHGNEATTNIT |
| | | ETTVKFTSTSVITSVYGNTNSSVQSQTSVISTVFTTPAN |
| | | VSTPETTLKPSLSPGNVSDLSTTSTSLATSPTKPYTSSS |
| | | PILSDIKAEIKCSGIREVKLTQGICLEQNKTSSCAEFKK |
| | | DRGEGLARVLCGEEQADADAGAQVCSLLLAQSEVRP |
| | | QCLLLVLANRTEISSKLQLMKKHQSDLKKLGILDFTE |
| | | QDVASHQSYSQKTLIALVTSGALLAVLGITGYFLMNR |
| | | RSWSPI |
| TrhCD34 | SEQ ID | VGPFEAMPRGWTALCLLSLLPSGFMSLDNNGTATPEL |
| | NO. 23 | PTQGTFSNVSTNVSYQETTTPSTLGSTSLHPVSQHGNE |
| | | ATTNITETTVKFTSTSVITSVYGNTNSSVQSQTSVISTV |
| | | FTTPANVSTPETTLKPSLSPGNVSDLSTTSTSLATSPTK |
| | | PYTSSSPILSDIKAEIKCSGIREVKLTQGICLEQNKTSSC |
| | | AEFKKDRGEGLARVLCGEEQADADAGAQVCSLLLA |
| | | QSEVRPQCLLLVLANRTEISSKLQLMKKHQSDLKKLG |
| | | ILDFTEQDVASHQSYSQKTLIALVTSGALLAVLGITGY |
| | | FLMNRRSWSP |
| P2A-hCD34 | SEQ ID | GSGATNFSLLKQAGDVEENPGPMPRGWTALCLLSLL |
| | NO: 24 | PSGFMSLDNNGTATPELPTQGTFSNVSTNVSYQETTT |
| | | PSTLGSTSLHPVSQHGNEATTNITETTVKFTSTSVITSV |
| | | YGNTNSSVQSQTSVISTVFTTPANVSTPETTLKPSLSP |
| | | GNVSDLSTTSTSLATSPTKPYTSSSPILSDIKAEIKCSGI |
| | | REVKLTQGICLEQNKTSSCAEFKKDRGEGLARVLCGE |
| | | EQADADAGAQVCSLLLAQSEVRPQCLLLVLANRTEIS |
| | | SKLQLMKKHQSDLKKLGILDFTEQDVASHQSYSQKT |
| | | LIALVTSGALLAVLGITGYFLMNRRSWSPI |
| P2A-TrhCD34 | SEQ ID | GSGATNFSLLKQAGDVEENPGPVGPFEAMPRGWTAL |
| | NO: 25 | CLLSLLPSGFMSLDNNGTATPELPTQGTFSNVSTNVS |
| | | YQETTTPSTLGSTSLHPVSQHGNEATTNITETTVKFTS |
| | | TSVITSVYGNTNSSVQSQTSVISTVFTTPANVSTPETTL |
| | | KPSLSPGNVSDLSTTSTSLATSPTKPYTSSSPILSDIKAE |
| | | IKCSGIREVKLTQGICLEQNKTSSCAEFKKDRGEGLAR |
| | | VLCGEEQADADAGAQVCSLLLAQSEVRPQCLLLVLA |
| | | NRTEISSKLQLMKKHQSDLKKLGILDFTEQDVASHQS |
| | | YSQKTLIALVTSGALLAVLGITGYFLMNRRSWSP |
| Human-Herpes | SEQ ID | MPRGWTALCLLSLLPSGFMSLDNNGTATPELPTQGTF |
| Simplex Virus-1 (HSV)- | NO: 26 | SNVSTNVSYQETTTPSTLGSTSLHPVSQHGNEATTNIT |
| thymidine kinase | | ETTVKFTSTSVITSVYGNTNSSVQSQTSVISTVFTTPAN |
| (TK) | | VSTPETTLKPSLSPGNVSDLSTTSTSLATSPTKPYTSSS |
| | | PILSDIKAEIKCSGIREVKLTQGICLEQNKTSSCAEFKK |
| | | DRGEGLARVLCGEEQADADAGAQVCSLLLAQSEVRP |
| | | QCLLLVLANRTEISSKLQLMKKHQSDLKKLGILDFTE |
| | | QDVASHQSYSQKTLIALVTSGALLAVLGITGYFLMNR |
| | | RSWSPTGEGGGGGDLGGVKLPHLFGKRLVEARMAS |
| | | YPCHQHASAFDQAARSRGHSNRRTALRPRRQQEATE |
| | | VRLEQKMPTLLRVYIDGPHGMGKTTTTQLLVALGSR |
| | | DDIVYVPEPMTYWQVLGASETIANIYTTQHRLDQGEI |
| | | SAGDAAVVMTSAQITMGMPYAVTDAVLAPHVGGEA |
| | | GSSHAPPPALTLLLDRHPIAVMLCYPAARYLMGSMTP |
| | | QAVLAFVALIPPTLPGTNIVLGALPEDRHIDRLAKRQR |
| | | PGERLDLAMLAAIRRVYGLLANTVRYLQGGGSWWE |
| | | DWGQLSGTAVPPQGAEPQSNAGPRPHIGDTLFTLFRA |
| | | PELLAPNGDLYNVFAWALDVLAKRLRPMHVFILDYD |
| | | QSPAGCRDALLQLTSGMVQTHVTTPGSIPTICDLARTF |
| | | AREMGEAN |

Table 5 below discloses the sequences of V_{H} and V_{L} domains which target the recited antigens. These sequences may be incorporated into CARs along with elements from Table 4 or as disclosed herein.

TABLE 5
Amino acid sequences of the variable heavy (V_{H}) and variable light (V_{L}) chains
of selected scFvs.

| | SEQ ID | |
|---|---|---|
| ScFv sequences | NO: | Amino acid sequence |
| CD2 heavy chain | SEQ ID | EVKLEESGAELVKPGASVKLSCRTSGFNIKDTYIHW |
| variable region (1) | NO: 27 | VKQRPEQGLKWIGRIDPANGNTKYDPKFQDKATVT |
| | | ADTSSNTAYLQLSSLTSEDTAVYYCVTYAYDGNWY |
| | | FDVWGAGTAVTVSS |
| CD2 light chain | SEQ ID | DIKMTQSPSSMYVSLGERVTITCKASQDINSFLSWFQ |
| variable region (1) | NO: 28 | QKPGKSPKTLIYRANRLVDGVPSRFSGSGSGQDYSLT |
| | | ISSLEYEDMEIYYCLQYDEFPYTFGGGTKLEMKR |
| CD2 heavy chain | SEQ ID | EVQLEESGAELVRPGTSVKLSCKASGYTFTSYWMH |
| variable region (2) | NO: 29 | WIKQRPEQGLEWIGRIDPYDSETHYNEKFKDKAILSV |
| | | DKSSSTAYIQLSSLTSDDSAVYYCSRRDAKYDGYAL |
| | | DYWGQGTSVTVSS |
| CD2 light chain | SEQ ID | DIVMTQAAPSVPVTPGESVSISCRSSKTLLHSNGNTY |
| variable region (2) | NO: 30 | LYWFLQRPGQSPQVLIYRMSNLASGVPNRFSGSGSE |
| | | TTFTLRISRVEAEDVGIYYCMQHLEYPYTFGGGTKLE |
| | | IER |
| CD3 heavy chain | SEQ ID | GSQVQLQQSGAELARPGASVKMSCKASGYTFTRYT |
| variable region | NO: 31 | MHWVKQRPGQGLEWIGYINPSRGYTNYNQKFKDK |
| (OKT3) | | ATLTTDKSSSTAYMQLSSLTSEDSAVYYCARYYDDH |
| | | YCLDYWGQGTTLTVSS |
| CD3 light chain | SEQ ID | QIVLTQSPAIMSASPGEKVTMTCSASSSVSYMNWYQ |
| variable region | NO: 32 | QKSGTSPKRWIYDTSKLASGVPAHFRGSGSGTSYSLT |
| (OKT3) | | ISGMEAEDAATYYCQQWSSNPFTFGSGTKLEINR |
| CD3 heavy chain | SEQ ID | EVQLVESGGGLVQPGGSLRLSCAASGYSFTGYTMN |
| variable region | NO: 33 | WVRQAPGKCLEWVALINPYKGVSTYNQKFKDRFTIS |
| (UCHT1) | | VDKSKNTAYLQMNSLRAEDTAVYYCARSGYYGDS |
| | | DWYFDVWGQGTLVTVSS |
| CD3 heavy chain | SEQ ID | DIQMTQSPSSLSASVGDRVTITCRASQDIRNYLNWY |
| variable region | NO: 34 | QQKPGKAPKLLIYYTSRLESGVPSRFSGSGSGTDYTL |
| (UCHT1) | | TISSLQPEDFATYYCQQGNTLPWTFGCGTKVEIK |
| CD7 heavy chain | SEQ ID | EVQLVESGGGLVKPGGSLKLSCAASGLTFSSYAMSW |
| variable region | NO: 35 | VRQTPEKRLEWVASISSGGFTYYPDSVKGRFTISRDN |
| | | ARNILYLQMSSLRSEDTAMYYCARDEVRGYLDVWG |
| | | AGTTVTVS |
| CD7 light chain | SEQ ID | DIQMTQTTSSLSASLGDRVTISCSASQGISNYLNWYQ |
| variable region | NO: 36 | QKPDGTVKLLIYYTSSLHSGVPSRFSGSGSGTDYSLTI |
| | | SNLEPEDIATYYCQQYSKLPYTFGGGTKLEIKR |
| FLT3 heavy chain | SEQ ID | EVQLVQSGAEVKKPGASVKVSCKASGYTFTSYYMH |
| variable region | NO: 37 | WVRQAPGQGLEWMGIINPSGGSTSYAQKFQGRVTM |
| | | TRDTSTSTVYMELSSLRSEDTAVYYCARGVGAHDAF |
| | | DIWGQGTTVTVSS |
| FLT3 light chain | SEQ ID | DVVMTQSPLSLPVTPGEPASISCRSSQSLLHSNGNNY |
| variable region | NO: 38 | LDWYLQKPGQSPQLLIYLGSNRASGVPDRFSGSGSD |
| | | TDFTLQISRVEAEDVGVYYCMQGTHPAISFGQGTRL |
| | | EIK |
| FLT3 heavy chain | SEQ ID | EVQLVQSGAEVKKPGSSVKVSCKASGGTFSSYAISW |
| variable region | NO: 39 | VRQAPGQGLEWMGGIIPIFGTANYAQKFQGRVTITA |
| | | DKSTSTAYMELSSLRSEDTAVYYCATFALFGFREQA |
| | | FDIWGQGTTVTVSS |
| FLT3 light chain | SEQ ID | DIQMTQSPSSLSASVGDRVTITCRASQSISSYLNWYQ |
| variable region | NO: 40 | QKPGKAPKLLIYAASSLQSGVPSRFSGSGSGTDFTLTI |
| | | SSLQPEDLATYYCQQSYSTPFTFGPGTKVDIK |
| FLT3 heavy chain | SEQ ID | EVQLVQSGAEVKKPGASVKVSCKASGYTFTSYYMH |
| variable region | NO: 41 | WARQAPGQGLEWMGIINPSGGSTSYAQKFQGRVTM |
| | | TRDTSTSTVYMELSSLRSEDTAVYYCARVVAAAVA |
| | | DYWGQGTLVTVSS |
| FLT3 light chain | SEQ ID | DVVMTQSPLSLPVTPGEPASISCRSSQSLLHSNGYNY |
| variable region | NO: 42 | LDWYLQKPGQSPQLLIYLGSNRASGVPDRFSGSGSG |
| | | TDFTLKISRVEAEDVGVYYCMQSLQTPFTFGPGTKV |
| | | DIK |
| CS1 heavy chain | SEQ ID | QVQLQQPGAELVRPGASVKLSCKASGYSFTTYWMN |
| variable region | NO: 43 | WVKQRPGQGLEWIGMIHPSDSETRL |
| | | NQKFKDKATLTVDKSSSTAYMQLSSPTSEDSAVYYC |
| | | ARSTMIATRAMDYWGQGTSVTVSS |
| CS1 light chain | SEQ ID | DIVMTQSQKSMSTSVGDRVSITCKASQDVITGVAWY |
| variable region | NO: 44 | QQKPGQSPKLLIYSASYRYTGVPD |
| | | RFTGSGSGTDFTFTISNVQAEDLAVYYCQQHYSTPLT |
| | | FGAGTKLELK |
| CD33 heavy chain | SEQ ID | QVQLQQPGAEVVKPGASVKMSCKASGYTFTSYYIH |
| variable region | NO: 45 | WIKQTPGQGLEWVGVIYPGNDDISYNQKFQGKATLT |
| | | ADKSSTTAYMQLSSLTSEDSAVYYCAREVRLRYFDV |
| | | WGQGTTVTVSSSG |
| CD33 light chain | SEQ ID | GSEIVLTQSPGSLAVSPGERVTMSCKSSQSVFFSSSQK |
| variable region | NO: 46 | NYLAWYQQIPGQSPRLLIYWASTRESGVPDRFTGSG |
| | | SGTDFTLTISSVQPEDLAIYYCHQYLSSRTFGQGTKL |
| | | EIKR |
| CD19 heavy chain | SEQ ID | LKPREVKLVESGGGLVQPGGSLKLSCAASGFDF |
| variable region | NO: 47 | SRYWMSWVRQAPGKGLEWIGEINLDSSTINYTP |
| | | SLKDKFIISRDNAKNTLYLQMSKVRSEDTALYY |
| | | CARRYDAMDYWGQGTSVTVSSAKTTAPSVYPL |
| | | APVCGDTTGSSVTLGCLVKASQ |
| CD19 light chain | SEQ ID | ASDIVLTQSPASLAVSLGQRATISCRASESVDDY |
| variable region | NO: 48 | GISFMNWFQQKPGQPPKLLIYAAPNQGSGVPAR |
| | | FSGSGSGTDFSLNIHPMEEDDTAMYFCQQSKDV |
| | | RWRHQAGDQTG |

### Cell-Specific Variations

The CAR components and construction methods disclosed above are generally suitable for use in T cells and other immune effector cells, but are not exhaustive. Certain variations may be useful in subsets of cells, and are known in the art.

For example, in NK cells, the TM domain may be chosen or adapted from NKG2D, FcγRIIIa, NKp44, NKp30, NKp46, actKIR, NKG2C, or CD8α. NK cells also express a number of transmembrane adapters that are triggered via association with activating receptors, providing an NK cell specific signal enhancement. For example, the TM adapter can be chosen or adapted from FceR1γ (ITAMx1), CD3ζ (ITAMx3), DAP12 (ITAMx1), or DAP10 (YxxM/YINM). In certain embodiments, the TM domains and adapters may be paired, e.g.: NKG2D and DAP10, FcγRIIIa and CD3ζ or FceR1γ, NKp44 and DAP12, NKp30 and CD3ζ or FceR1γ, NKp46 and CD3ζ or FceR1γ, actKIR and DAP12, and NKG2C and DAP12.

In certain embodiments, in NK cells, the hinge domain may be chosen or adapted from, e.g., NKG2, TMα, or CD8.

In certain embodiments, in NK cells, the intracellular signaling and/or costimulatory domain may comprise one or more of: CD137/41BB (TRAF, NFkB), DNAM-1 (Y-motif), NKp80 (Y-motif), 2B4 (SLAMF)::ITSM, CRACC (CS1/SLAMF7):: ITSM, CD2 (Y-motifs, MAPK/Erk), CD27 (TRAF, NFkB); one or more integrins (e.g., multiple integrins); a cytokine receptor associated with persistence, survival, or metabolism, such as IL-2/15Rbyc::Jak1/3, STAT3/5, PI3K/mTOR, and MAPK/ERK; a cytokine receptor associated with activation, such as IL-18R::NFkB. a cytokine receptor associated with IFN-y production, such as IL-12R::STAT4; a cytokine receptor associated with cytotoxicity or persistence, such as IL-21R::Jak3/Tyk2, or STAT3; and a TM adapter, as disclosed above. In some embodiments, the NK cell CAR comprises three signaling domains, a TM domain, and optionally, a TM adapter.

The choice of costimulatory domain may also depend on the phenotype or subtype of the NK cell; for example, in some experiments, 4-1BB may be effective as a costimulatory domain in memory-like (ML) NK cells (including CIMLs) but less efficacious in NK cells. Additionally, signaling domains that may be harnessed that are more selectively expressed in ML NK cells include DNAM-1, CD137, and CD2.

### Immune Effector Cells

Immune effector cells as disclosed herein include NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells.

### NK Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

Memory, Memory-Like, and CIML NK cells

In addition to their innate cytotoxic and immunostimulatory activity, NK cells constitute a heterogeneous and versatile cell subset, including persistent memory NK populations, in some cases also called memory-like or cytokine-induced-memory-like (CIML) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+ NK cells were reduced. Finally, unlike in vitro differentiated ML NK cells, in vivo differentiated ML NK cells did not express CD25.

### Cytokine-Induced Memory-Like Natural Killer Cells (CIML-NKs)

NK cells may be induced to acquire a memory-like phenotype, for example by priming (preactivation) with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8 (357): 357). Memory NK cells may also be identified by observed in vitro and in vivo properties, such as enhanced effector functions such as cytotoxicity, improved persistence, increased IFN-γ production, and the like, when compared to a heterogenous NK cell population.

### Pharmaceutical Compositions

Also disclosed is a pharmaceutical composition comprising a disclosed molecule in a pharmaceutically acceptable carrier. Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Treatment Applications

NK Cells disclosed herein can be used in the treatment or prevention of progression of proliferative diseases such as cancers and myelodysplastic syndromes. The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), non-Hodgkin's lymphomas, B-cell lymphomas, T-cell lymphomas, mantle cell lymphomas, Burkitt's lymphoma, follicular lymphoma, and others as defined herein and known in the art. Myelodysplastic syndromes comprise a group of diseases affecting immature leukocytes and/or hematopoietic stem cells (HSCs); MDS may progress to AML.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

Solid tumors include melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

Methods described herein are generally performed on a subject in need thereof. A subject in need of the therapeutic methods described herein can be a subject having. diagnosed with, suspected of having, or at risk for developing, or at rick of progressing to a later stage of, cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans, or other animals such as chickens. For example, the subject can be a human subject.

Generally, a safe and effective amount of a therapy, e.g., an antibody or functional antigen-binding fragment thereof, CAR-bearing immune effector cell, or antibody-drug conjugate, is, for example, an amount that would cause the desired therapeutic effect in a subject while minimizing undesired side effects.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. Where the product is, for example, a biologic or cell therapy, the mode of administration will likely be via injection or infusion.

### Standards of Care and Conditioning Regimens for Immunotherapy

Standard of care treatment for cancers, such as AML, can involve anti-cancer pharmaceutical therapy including chemotherapy and targeted therapy.

For example, the combination of cytarabine (cytosine arabinoside or ara-C) and an anthracycline such as daunorubicin (daunomycin) or idarubicin is the first-line chemotherapy for AML. Other chemotherapeutics that may be used to treat AML include cladribine (Leustatin, 2-CdA), fludarabine (Fludara), mitoxantrone, Etoposide (VP-16), 6-thioguanine (6-TG), hydroxyurea, corticosteroids such as prednisone or dexamethasone, methotrexate (MTX), 6-mercaptopurine (6-MP), azacitidine (Vidaza), and decitabine (Dacogen). In addition, targeted therapies may be used in appropriate patients, such as midostaurin (Rydapt) or gilteritinib (Xospata) in patients with FLT-3 mutations; gemtuzumab ozogamicin (Mylotarg) in CD33-positive AML; BCL-2 inhibitor such as venetoclax (Venclexta); IDH inhibitors such as ivosidenib (Tibsovo) or enasidenib (Idhifa); and hedgehog pathway inhibitors such as glasdegib (Daurismo). Although the rate of complete remission can be as high as 80% following initial induction chemotherapy, the majority of AML patients will eventually progress to relapsed or refractory (RR) disease, and five-year survival rate are about 35% in people under 60 years old and 10% in people over 60 years old. See, Walter RB et al., “Resistance prediction in AML: analysis of 4601 patients from MRC/NCRI, HOVON/SAKK, SWOG and MD Anderson Cancer Center,” Leukemia 29(2):312-20 (2015) and Döhner, Het al., “Acute Myeloid Leukemia,” NEJM 373 (12): 1136-52 (2015).

Adoptive cell transfer (ACT) therapy is possible in the treatment of cancers either with or without a conditioning regimen. Typically, when ACT such as HSCT is performed in patients with malignant disorders, preparative or conditioning regimens are administered as part of the procedure to effect immunoablation to prevent graft rejection, and to reduce tumor burden. Traditionally, these goals have been achieved by using otherwise supralethal doses of total body irradiation (TBI) and chemotherapeutic agents with nonoverlapping toxicities, so-called “high-intensity” pre-ACT conditioning. However, as it was recognized that immunologic reactions of donor cells against malignant host cells (i.e., graft-versus-tumor effects) substantially contributed to the effectiveness of ACT, reduced-intensity and nonmyeloablative conditioning regimens have been developed, making ACT applicable to a wider variety of patients, including older and medically infirm patients.

Conditioning regimens are known in the art. See, e.g., Gyurkocza and Sandmaier B M, “Conditioning regimens for hematopoietic cell transplantation: one size does not fit all,” Blood 124(3): 344-353 (2014). Conditioning regimens may be classified as high-dose (myeloablative), reduced-intensity, and nonmyeloablative, following the Reduced-Intensity Conditioning Regimen Workshop, convened by the Center for International Blood and Marrow Transplant Research (CIBMTR) during the Bone Marrow Transplantation Tandem Meeting in 2006.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

As used herein, the term “antibody” refers to a polypeptide that includes canonical immunoglobulin sequence elements sufficient to confer specific binding, or e.g., immune-reacts and/or is directed to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{H}) domain, followed by three constant domains: C_{H}1, C_{H}2, and the carboxy-terminal C_{H}3. A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “Complementarity-Determining Regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity.

An “antibody fragment” refers to a molecule other than an intact antibody that comprises a portion of an intact antibody that binds the antigen to which the intact antibody binds. Several examples of antibody fragments include but are not limited to Fv, Fab, Fab′, Fab′-SH, F (ab′)₂, diabodies, linear antibodies, single chain variable fragments (scFvs), and multi-specific antibodies formed from antibody fragments. In some embodiments, the antibody fragment is an antigen-binding fragment.

Reviews of current methods for antibody engineering and improvement can be found in R. Kontermann and S. Dubel, (2010) Antibody Engineering Vols. 1 and 2, Springer Protocols, 2^{nd} Edition and W. Strohl and L. Strohl (2012) Therapeutic antibody engineering: Current and future advances driving the strongest growth area in the pharmaceutical industry, Woodhead Publishing. Methods for producing and purifying antibodies and antigen-binding fragments are well known in the art and can be found, in Harlow and Lane (1988) Antibodies: A Laboratory Manual, Cold Spring Harbor Laboratory Press, Cold Spring Harbor, NY, chapters 5-8 and 15.

The term “antigen” refers to a molecular entity that may be soluble or cell membrane bound in particular but not restricted to molecular entities that can be recognized by means of the adaptive immune system including but not restricted to antibodies or TCRs, or engineered molecules including but not restricted to transgenic TCRs, chimeric antigen receptors (CARs), scFvs or multimers thereof, Fab-fragments or multimers thereof, antibodies or multimers thereof, single chain antibodies or multimers thereof, or any other molecule that can execute binding to a structure with high affinity.

An “antigen binding domain” as used herein, in the context of a CAR, refers to the region of the CAR that specifically binds to an antigen (and thereby is able to target a cell containing an antigen). CARs may comprise one or more antigen binding domains. Generally, the targeting regions on the CAR are extracellular. The antigen binding domain may comprise an antibody or an antigen-binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable portions of an immunoglobulin heavy chain and light chain are fused by a flexible linker to form a scFv. Such a linker may be for example the (GGGG₄S)₃. In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen-binding fragment thereof. Human or humanized antibodies or fragments thereof can be made by a variety of methods well known in the art.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

The term “cancer” is known medically as a malignant neoplasm. Cancer is a broad group of diseases involving upregulated cell growth. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The term “chemotherapy” refers to the treatment of cancer (cancerous cells) with one or more cytotoxic anti-neoplastic drugs (“chemotherapeutic agents” or “chemotherapeutic drugs”) as part of a standardized regimen. Chemotherapy may be given with a curative intent or it may aim to prolong life or to palliate symptoms. It is often used in conjunction with other cancer treatments, such as radiation therapy, surgery, and/or hyperthermia therapy. Traditional chemotherapeutic agents act by killing cells that divide rapidly, one of the main properties of most cancer cells. This means that chemotherapy also harms cells that divide rapidly under normal circumstances, such as cells in the bone marrow, digestive tract, and hair follicles. This results in the most common side-effects of chemotherapy, such as myelosuppression (decreased production of blood cells, hence also immunosuppression), mucositis (inflammation of the lining of the digestive tract), and alopecia (hair loss).

The term “chimeric antigen receptor,” abbreviated “CAR,” refers to engineered receptors, which graft an antigen specificity onto cells, for example T or NK cells. The CARs disclosed herein comprise an antigen binding domain also known as antigen targeting region (typically a single chain variable region comprised of antibody heavy and light chain variable regions), an extracellular spacer/linker domain or hinge region, a transmembrane domain and at least one intracellular signaling domain; it may optionally comprise other elements, such as at least one co-stimulatory domain. The extracellular domain may also comprise a signal peptide. Upon binding of the antigen-specific region to the corresponding antigen, the signaling domain mediates an effector cell function in the host cell.

The term “combination immunotherapy” refers to the concerted application of two therapy approaches e.g., therapy approaches known in the art for the treatment of disease such as cancer. The term “combination immunotherapy” may also refer to the concerted application of an immunotherapy such as the treatment with an antigen recognizing receptor and another therapy such as the transplantation of hematopoietic cells e.g., hematopoietic cells resistant to recognition by the antigen recognizing receptor. Expression of an antigen on a cell means that the antigen is sufficient present on the cell surface of the cell, so that it can be detected, bound and/or recognized by an antigen-recognizing receptor.

The “costimulatory signaling region” (equivalently, costimulatory or “co-stim” domain) refers to a part of the CAR comprising the intracellular domain of a costimulatory molecule. A costimulatory molecule is a cell surface molecule other than an antigen receptor or their ligands that is required for efficient response of immune effector cells. Examples for a costimulatory molecule discussed above and known in the art. A short oligo-or polypeptide linker, which is typically between 2 and 10 amino acids in length, may form the linkage between elements of the intracellular signaling domain. A prominent linker is the glycine-serine doublet.

The term “cytokine-induced memory-like,” or, equivalently, “CIML,” in reference to NK cells, means having a “memory” or “memory-like” phenotype and produced using a priming agent.

The term “cytotoxicity,” as used herein in reference to memory NK cells, refers to the ability of cells to target and kill diseased cells.

A “diseased cell” refers to the state of a cell, tissue or organism that diverges from the normal or healthy state and may result from the influence of a pathogen, a toxic substance, irradiation, or cell internal deregulation. A “diseased cell” may also refer to a cell that has been infected with a pathogenic virus. Further the term “diseased cell” may refer to a malignant cell or neoplastic cell that may constitute or give rise to cancer in an individual.

The terms “engineered cell” and “genetically modified cell” as used herein can be used interchangeably. The terms mean containing and/or expressing a foreign gene or nucleic acid sequence, or containing a gene which has been genetically modified to deviate from its natural form or function (for example a deleted or knocked-out gene) which in turn modifies the genotype or phenotype of the cell or its progeny. Cells can be modified by recombinant methods well known in the art to express stably or transiently peptides or proteins, which are not expressed in these cells in the natural state. Methods of genetic modification of cells may include but is not restricted to transfection, electroporation, nucleofection, transduction using retroviral vectors, lentiviral vectors, non-integrating retro-or lentiviral vectors, transposons, designer nucleases including zinc finger nucleases, TALENs or CRISPR/Cas.

The term “enrich” as used herein in relation to NK cells means to concentrate, purify, or isolate for further analysis or use. Enriched and purified cell populations comprise a majority of the desired cell, and a negligible fraction of other cells.

The term “fold selective,” as used herein, means having an affinity for one target that is at least x-fold greater than its affinity for another target, wherein x is at least 2, and may be higher, e.g., 10, 20, 50, 100, or 1000. In preferred embodiments, the fold selectivity is therapeutically meaningful, i.e., sufficient to permit cells expressing one target to be killed and cells bearing the other target to be spared.

The term “genetic modification” or genetically modified” refers to the alteration of the nucleic acid content including but not restricted to the genomic DNA of a cell. This includes but is not restricted to the alteration of a cells genomic DNA sequence by introduction exchange or deletion of single nucleotides or fragments of nucleic acid sequence. The term also refers to any introduction of nucleic acid into a cell independent of whether that leads to a direct or indirect alteration of the cells genomic DNA sequence or not.

The term “hematopoietic cells”, refers to a population of cells of the hematopoietic lineage capable of hematopoiesis which include but is not limited to hematopoietic stem cells and/or hematopoietic progenitor cells (i.e., capable to proliferate and at least partially reconstitute different blood cell types, including erythroid cells, lymphocytes, and myelocytes). The term “hematopoietic cells” as used herein also includes the cells that are differentiated from the hematopoietic stem cells and/or hematopoietic progenitor cells to form blood cells (i.e., blood cell types, including erythroid cells, lymphocytes, and myelocytes).

A donor hematopoietic cell resistant to recognition of an antigen by an antigen-recognizing receptor means that the cell cannot as easily be detected, bound and/or recognized by an antigen-recognizing receptor specific for the antigen or that the detection, binding and/or recognizing is impaired, so the cell is not killed during immunotherapy.

The term “immune cell” or “immune effector cell” refers to a cell that may be part of the immune system and executes a particular effector function such as alpha-beta T cells, NK cells (including memory NKs, ML-NKs, and CIML-NKs), NKT cells (including iNKT cells), B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes and macrophages. Preferred immune cells are cells with cytotoxic effector function such as alpha-beta T cells, NK cells (including memory NKs, ML-NKs, and CIML-NKs), NKT cells (including iNKT cells), ILC, CIK cells, LAK cells or gamma-delta T cells. “Effector function” means a specialized function of a cell, e.g., in an NK cell an effector function may be cytolytic activity or helper activity including the secretion of cytokines.

The term “immunotherapy” is a medical term defined as the “treatment of disease by inducing, enhancing, or suppressing an immune response” Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based cytotoxic responses to attack cancer cells Immune cells such as T cells that have a natural or genetically engineered reactivity to a patient's cancer are generated in vitro and then transferred back into the cancer patient.

As used herein, the term “individual” refers to an animal. Preferentially, the individual is a mammal such as mouse, rat, cow, pig, goat, chicken dog, monkey or human. More preferentially, the individual is a human. The individual may be an individual suffering from a disease such as cancer (a patient), but the subject may be also a healthy subject.

The “intracellular signaling domain” (equivalently, cytoplasmic signalling domain or effector domain; which are part of the intracellular or endodomain) of a CAR is responsible for activation of at least one of the normal effector functions of the immune cell in which the CAR is expressed. “Effector function” means a specialized function of a cell, e.g. in an NK cell an effector function may be cytolytic activity or helper activity including the secretion of cytokines. The intracellular signaling domain refers to the part of a protein which transduces the effector function signal and directs the cell expressing the CAR to perform a specialized function.

The intracellular signaling domain may include any complete or truncated part of the intracellular signaling domain of a given protein sufficient to transduce the effector function signal. Prominent examples of intracellular signaling domains for use in the CARs include the cytoplasmic sequences of receptors and co-receptors that act in concert to initiate signal transduction following antigen receptor engagement.

Generally, CAR activation of immune effector cells can be mediated by two classes of cytoplasmic signaling sequences, firstly those that initiate antigen-dependent primary activation through the CAR (primary cytoplasmic signaling sequences) and secondly those that act in an antigen-independent manner to provide a secondary or co-stimulatory signal (secondary cytoplasmic signaling sequences, costimulatory signaling domain). Therefore, an intracellular signaling domain of a CAR may comprise a primary cytoplasmic signaling domain and optionally a secondary cytoplasmic signaling domain (i.e., a costimulatory or “co-stim” domain).

Primary cytoplasmic signaling sequences that act in a stimulatory manner may contain ITAMs (immunoreceptor tyrosine-based activation motifs signaling motifs). Examples of ITAM containing primary cytoplasmic signaling sequences often used in CARs are disclosed herein and known in the art.

The term “malignant” or “malignancy” describes cells, groups of cells or tissues that constitute a neoplasm, are derived from a neoplasm or can be the origin of new neoplastic cells. The term is used to describe neoplastic cells in contrast to normal or healthy cells of a tissue. A malignant tumor contrasts with a non-cancerous benign tumor in that a malignancy is not self-limited in its growth, is capable of invading into adjacent tissues, and may be capable of spreading to distant tissues. A benign tumor has none of those properties. Malignancy is characterized by anaplasia, invasiveness, and metastasis as well as genome instability. The term “premalignant cells” refer to cells or tissue that is not yet malignant but is poised to become malignant.

The term “memory” or “memory-like,” in reference to NK cells, means having an activated phenotype with improved cytotoxicity and longevity/persistence compared to a general population of NK cells, and typically exhibits increased cell-surface expression of CD69, CD25, and NKG2A, and maintained expression of CD16, compared to a general population of NK cells.

The term “monoclonal antibody” (mAb), as applied to the antibodies described in the present disclosure, are compounds derived from a single copy or a clone from any eukaryotic, prokaryotic, or phage clone, and not the method by which it is produced. mAbs of the present disclosure may exist in a homogeneous or substantially homogeneous population.

The term “persistence” as sued herein refers to the ability of cells, especially adoptively transferred into a subject, to continue to live.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “prime,” in reference to NK cells, means to stimulate or activate into a memory/memory-like phenotype using a priming agent. A “priming agent” comprises a combination of stimulatory cytokines, for example,
one or more of IL-12, IL-23, IL-27, and IL-35;
one or more of IL-2, IL-4, IL-7, IL-9, IL-15, and IL-21; and
one or more of IL-18, IL-1a, IL-1b, IL-36a, IL-36b, and IL-36g,
   or one or more “priming fusion proteins” comprising functional fragments of such cytokines, or one or more multichain complexes thereof. Examples of such proteins are disclosed herein.

In general, the term “receptor” refers to a biomolecule that may be soluble or attached to the cell surface membrane and specifically binds a defined structure that may be attached to a cell surface membrane or soluble. Receptors include but are not restricted to antibodies and antibody like structures, adhesion molecules, transgenic or naturally occurring TCRs or CARs. In specific, the term “antigen-recognizing receptor” as used herein may be a membrane bound or soluble receptor such as a natural TCR, a transgenic TCR, a CAR, a scFv or multimers thereof, a Fab-fragment or multimers thereof, an antibody or multimers thereof, a bi-specific T cell enhancer (BiTE), a diabody, or any other molecule that can execute specific binding with high affinity.

The term “reducing side-effects” refers to the decrease of severity of any complication, unwanted or pathological outcome of an immunotherapy with an antigen recognizing receptor such as toxicity towards an antigen-expressing non-target cell. “Reducing side-effects” also refers to measures that decrease or avoid pain, harm or the risk of death for the patient during the immunotherapy with an antigen recognizing receptor.

As used herein, the term “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods. Methods to determine identity are designed to give the largest match between the sequences tested. Moreover, methods to determine identity are codified in publicly available computer programs. Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc, San Diego, California, United States of America), or by visual inspection. See generally, Altschul, S. F. et al., J. Mol. Biol. 215: 403-410 (1990) and Altschul et al. Nucl. Acids Res. 25: 3389-3402 (1997). One example of an algorithm that is suitable for determining percent sequence identity and sequence similarity is the BLAST algorithm,

A “signal peptide” as used herein, in the context of a CAR, refers to a peptide sequence that directs the transport and localization of the protein within a cell, e.g. to a certain cell organelle (such as the endoplasmic reticulum) and/or the cell surface.

The term “spacer” or “hinge” as used herein, in the context of a CAR, refers to the hydrophilic region which is between the antigen binding domain and the transmembrane domain. The CARs disclosed herein may comprise an extracellular spacer domain but is it also possible to pass such a spacer. The spacer may include Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The terms “specifically binds” or “specific for” or “specifically recognize” with respect to an antigen-recognizing receptor refer to an antigen-binding domain of the antigen-recognizing receptor which recognizes and binds to a specific polymorphic variant of an antigen, but does not substantially recognize or bind other variants.

The term “side-effects” refers to any complication, unwanted or pathological outcome of an immunotherapy with an antigen recognizing receptor that occurs in addition to the desired treatment outcome. The term “side effect” preferentially refers to on-target off-tumor toxicity, that might occur during immunotherapy in case of presence of the target antigen on a cell that is an antigen-expressing non-target cell but not a diseased cell as described herein. A side-effect of an immunotherapy may be the developing of graft versus host disease.

The term “target” or “target antigen” refers to any cell surface protein, glycoprotein, glycolipid or any other structure present on the surface of the target cell. The term also refers to any other structure present on target cells in particular but not restricted to structures that can be recognized by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “target cells” as used herein refers to cells which are recognized by the antigen-recognizing receptor which is or will be applied to the individual.

The term “therapeutically effective amount” means an amount which provides a therapeutic benefit.

The “transmembrane domain” of the CAR can be derived from any desired natural or synthetic source for such domain. When the source is natural the domain may be derived from any membrane-bound or transmembrane protein. The transmembrane domain may be derived for example from CD8alpha, CD28, NKG2D, or others disclosed herein or known in the art. When the key signaling and antigen recognition modules are on two (or even more) polypeptides then the CAR may have two (or more) transmembrane domains. Splitting key signaling and antigen recognition modules enables for a small molecule-dependent, titratable and reversible control over CAR cell expression (Wu et al, 2015, Science 350: 293-303) due to small molecule-dependent heterodimerizing domains in each polypeptide of the CAR.

As used herein, the term “transplant” means administering to a subject a population of donor cells, e.g. hematopoietic cells or CAR-bearing immune effector cells.

The term “treatment” as used herein means to reduce the frequency or severity of at least one sign or symptom of a disease.

### EXAMPLES

The invention is further illustrated by the following examples.

### Example 1. In Vitro Culturing and Activity of Expand Only, Prime While Expand, and Expand Then Prime

Material and Method: NK cells were isolated from whole blood using CD3 depletion and CD56 positive selection. NK cells selected were then cultured in 96-well plates in NK MACS media+supplements+10% HI-HAB, and were primed/expanded in the following conditions, where 1×7t15-21s and ATF1 is 200 nM and 100 nM respectively, and 1×18t15-12s is 250 nM; all dilutions are calculated from these values as indicated.
a) Expand Only: +7t15-21s and ATF1 for either 2, 6, or 10 days in 37deg, 5% CO2 at the indicated concentrations. Every 2 days following, 7t15-21s and ATF1 were replenished to the indicated concentrations with fresh media.
b) Prime while Expand: +18t15-12s, 7t15-21s and ATF1 for either 2, 6, or 10 days in 37deg, 5% CO2 at the indicated concentrations. Every 2 days following, 18t15-12s, 7t15-21s and ATF1 were replenished to the indicated concentrations with fresh media.
c) Expand then Prime: +7t15-21s and ATF1 for either 2, 6, or 10 days in 37deg, 5% CO2 at the indicated concentrations. Every 2 days following, 7t15-21s and ATF1 were replenished to the indicated concentrations with fresh media. At day 2, 6, or 10 as indicated, 18t15-12s was added at the indicated concentration overnight.

To assess the phenotype of the NK cells generated by the above processes, at the appropriate timepoint, NK cells were harvested, washed, and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones were used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)
   An Attune NXt flow cytometer was used. Data were then analyzed in Flowjo v10.7, gating on Live CD56+CD3-cells and assessing the median fluorescence intensity of each of the above-described markers. Increases in CD69, CD25, and NKG2A expression, and maintenance of CD16 expression, indicates a CIML-NK cell phenotype.

To assess killing activity of the NK cells generated by the above processes, at the appropriate timepoint, Cultured NK cells were harvested and washed, then resuspended in NK MACS Media with 10% human AB serum (Gibco)) and added to a 96-well plate with 10,000 Luciferase expressing K562 (K562-Luc) human tumor cells (ATCC) at the indicated effector to target (E:T) ratios 24-48 hours, with or without IL-2 (Miltenyi), after which luciferase activity (live K562 cells) was assessed by luciferase readout (Promega). Data not shown.

Result: This example demonstrates the in vitro activity and flow cytometry phenotype of NK cells generated by the above processes.

Results are shown in Tables 6-11, showing cumulative fold change in surface protein expression, cell size, and median fluorescence intensities for individual genes.

TABLE 6
Average of Cumulative Fold Change

| | [18t15-12s on Y-axis where 1x = 250 nM; | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 2 | 4 | 2 | 6 | 2 | 5 | 2 | 5 |
| X/4 | 3 | 2 | 3 | 2 | 1 | 1 | 2 | 2 |
| X/16 | 3 | 1 | 1 | 2 | 1 | 6 | 6 | 2 |
| X/64 | 1 | 4 | 4 | 1 | 5 | 5 | 1 | 6 |
| X/256 | 2 | 1 | 1 | 6 | 5 | 1 | 6 | 4 |
| 0 | 4 | 4 | 2 | 4 | 4 | 2 | 2 | 2 |
| D 6 | | | | | | | | |
| 1X | 111 | 52 | 19 | 45 | 36 | 30 | 11 | 56 |
| X/4 | 28 | 9 | 7 | 10 | 11 | 12 | 22 | 9 |
| X/16 | 14 | 9 | 8 | 6 | 5 | 4 | 7 | 5 |
| X/64 | 17 | 7 | 5 | 5 | 4 | 5 | 4 | 4 |
| X/256 | 6 | 7 | 5 | 4 | 5 | 6 | 5 | 3 |
| 0 | 12 | 7 | 5 | 10 | 8 | 6 | 6 | 4 |
| D 10 | | | | | | | | |
| 1X | 5356 | 4444 | 2600 | 3394 | 6192 | 4006 | 4129 | 6753 |
| X/4 | 331 | 714 | 344 | 304 | 161 | 407 | 605 | 36 |
| X/16 | 199 | 85 | 27 | 37 | 34 | 23 | 78 | 16 |
| X/64 | 44 | 22 | 18 | 37 | 17 | 28 | 12 | 11 |
| X/256 | 45 | 60 | 36 | 26 | 13 | 12 | 11 | 6 |
| 0 | 98 | 41 | 26 | 35 | 26 | 25 | 62 | 7 |
| Expand Only D 2 | 2 | 2 | 2 | 3 | 3 | 2 | 3 | 3 |
| Expand Only D 6 | 18 | 14 | 10 | 13 | 16 | 12 | 12 | 6 |
| Expand Only D 10 | 236 | 72 | 56 | 33 | 44 | 49 | 53 | 19 |
| Expand then Prime D 2 | | | | | | | | |
| 1X | 2 | 1 | 1 | 1 | 1 | 2 | 1 | 2 |
| X/4 | 1 | 2 | 1 | 1 | 1 | 2 | 1 | 1 |
| X/16 | 1 | 1 | 2 | 2 | 1 | 1 | 1 | 1 |
| X/64 | 1 | 1 | 2 | 1 | 2 | 1 | 1 | 2 |
| X/256 | 1 | 1 | 3 | 2 | 1 | 1 | 1 | 2 |
| 0 | 1 | 2 | 2 | 2 | 1 | 2 | 2 | 2 |
| Expand then Prime D 6 | | | | | | | | |
| 1X | 26 | 7 | 7 | 3 | 6 | 3 | 7 | 3 |
| X/4 | 11 | 3 | 5 | 4 | 7 | 7 | 6 | 3 |
| X/16 | 7 | 6 | 4 | 5 | 8 | 5 | 4 | 1 |
| X/64 | 16 | 19 | 4 | 5 | 4 | 6 | 3 | 3 |
| X/256 | 29 | 9 | 13 | 5 | 5 | 6 | 5 | 2 |
| 0 | 28 | 10 | 5 | 7 | 5 | 4 | 9 | 4 |
| Expand then Prime D 10 | | | | | | | | |
| 1X | 332 | 71 | 130 | 37 | 90 | 82 | 70 | 31 |
| X/4 | 105 | 85 | 90 | 58 | 107 | 107 | 58 | 47 |
| X/16 | 81 | 59 | 73 | 51 | 43 | 167 | 74 | 7 |
| X/64 | 166 | 254 | 39 | 45 | 32 | 108 | 66 | 5 |
| X/256 | 405 | 68 | 61 | 36 | 65 | 30 | 33 | 12 |
| 0 | 381 | 146 | 22 | 57 | 73 | 52 | 139 | 15 |

TABLE 7
Cell Size (FSC)

| | [18t15-12s on Y-axis where 1x = 250 nM | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 211883 | 212139 | 208811 | 215637 | 213931 | 218368 | 211029 | 213845 |
| X/4 | 214784 | 214187 | 222635 | 214699 | 207445 | 217259 | 221184 | 219563 |
| X/16 | 211968 | 212907 | 208128 | 212395 | 205568 | 220928 | 222805 | 215808 |
| X/64 | 211712 | 215381 | 216747 | 214528 | 219392 | 218624 | 213675 | 216917 |
| X/256 | 209323 | 210517 | 205397 | 218880 | 217515 | 208896 | 213589 | 210261 |
| 0 | 207531 | 205141 | 207104 | 205995 | 207445 | 203349 | 198997 | 180309 |
| D 6 | | | | | | | | |
| 1X | 416667 | 367168 | 396944 | 370520 | 321677 | 297947 | 376197 | 370808 |
| X/4 | 365176 | 301595 | 322560 | 396435 | 332485 | 333917 | 385963 | 355669 |
| X/16 | 368896 | 336037 | 309248 | 322920 | 326656 | 281088 | 350208 | 319659 |
| X/64 | 355363 | 290816 | 226133 | 265813 | 287744 | 461123 | 379205 | 280832 |
| X/256 | 362496 | 436133 | 411536 | 341504 | 278187 | 233301 | 303189 | 236629 |
| 0 | 304213 | 267520 | 281600 | 298325 | 296704 | 273323 | 244651 | 194560 |
| D 10 | | | | | | | | |
| 1X | 381013 | 323243 | 369493 | 353707 | 346368 | 328107 | 337664 | 333909 |
| X/4 | 312149 | 292352 | 282880 | 239701 | 246699 | 295765 | 277931 | 281429 |
| X/16 | 266325 | 236288 | 218539 | 244309 | 260267 | 279381 | 225536 | 204544 |
| X/64 | 241493 | 195072 | 194901 | 232533 | 237995 | 274091 | 205824 | 203520 |
| X/256 | 246016 | 233387 | 236800 | 241152 | 194645 | 175957 | 240811 | 191403 |
| 0 | 296533 | 266752 | 261291 | 272043 | 291669 | 273152 | 269312 | 237739 |
| Expand Only D 2 | 205511 | 207275 | 208882 | 208839 | 208000 | 205028 | 198599 | 180793 |
| Expand Only D 6 | 306887 | 290176 | 293776 | 284672 | 276636 | 293175 | 255545 | 240412 |
| Expand Only D 10 | 296092 | 281870 | 272057 | 258133 | 268572 | 277618 | 258133 | 210588 |
| Expand then Prime D 2 | | | | | | | | |
| 1X | 290560 | 278699 | 267349 | 256341 | 247637 | 222635 | 222123 | 181163 |
| X/4 | 305493 | 278869 | 264192 | 259755 | 281856 | 263424 | 210005 | 183211 |
| X/16 | 297728 | 285525 | 260949 | 266496 | 249259 | 261461 | 215723 | 187819 |
| X/64 | 280576 | 274091 | 281003 | 260352 | 248576 | 237056 | 221867 | 187648 |
| X/256 | 288000 | 289195 | 275541 | 255403 | 254208 | 238421 | 219819 | 185344 |
| 0 | 263509 | 262912 | 249771 | 242859 | 226048 | 215296 | 204800 | 175531 |
| Expand then Prime D 6 | | | | | | | | |
| 1X | 313600 | 287232 | 261717 | 283136 | 270677 | 258048 | 232107 | 190891 |
| X/4 | 273664 | 303787 | 264021 | 225877 | 267264 | 273749 | 295765 | 217259 |
| X/16 | 296021 | 285440 | 288597 | 278869 | 286208 | 260181 | 248747 | 189952 |
| X/64 | 326827 | 331605 | 243541 | 284928 | 232533 | 284160 | 224512 | 196181 |
| X/256 | 338944 | 272469 | 288683 | 293803 | 242944 | 289621 | 257365 | 205312 |
| 0 | 346539 | 277845 | 224427 | 266752 | 265216 | 265216 | 273493 | 197035 |
| Expand then Prime D 10 | | | | | | | | |
| 1X | 286976 | 263339 | 271872 | 243200 | 273920 | 257963 | 284757 | 199680 |
| X/4 | 252245 | 277675 | 271104 | 295168 | 268032 | 325632 | 272811 | 267789 |
| X/16 | 284587 | 301739 | 286891 | 294656 | 246955 | 289963 | 279467 | 212309 |
| X/64 | 304640 | 303787 | 256512 | 308139 | 262656 | 294485 | 288597 | 249600 |
| X/256 | 294400 | 279808 | 299861 | 267179 | 314197 | 308309 | 272981 | 265899 |
| 0 | 297557 | 281685 | 299691 | 260608 | 262656 | 264021 | 265728 | 251136 |

TABLE 8
CD25 MFI

| | [18t15-12s on Y-axis where 1x = 250 nM | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 8692 | 6855 | 7126 | 6939 | 7021 | 6821 | 6647 | 6775 |
| X/4 | 4238 | 3914 | 3128 | 3064 | 2728 | 2833 | 3021 | 3471 |
| X/16 | 3557 | 2694 | 2202 | 2128 | 1784 | 1947 | 2083 | 2041 |
| X/64 | 2968 | 2554 | 2154 | 1465 | 1709 | 1876 | 1425 | 1388 |
| X/256 | 3296 | 2358 | 1936 | 1554 | 1569 | 1186 | 1169 | 1114 |
| 0 | 3445 | 2130 | 1648 | 1426 | 976 | 910 | 851 | 352 |
| D 6 | | | | | | | | |
| 1X | 44986 | 31964 | 37268 | 32888 | 33553 | 33259 | 28672 | 26932 |
| X/4 | 26737 | 21076 | 18766 | 20455 | 23874 | 19112 | 16521 | 14900 |
| X/16 | 18279 | 18862 | 9264 | 2003 | 12937 | 8265 | 9018 | 6723 |
| X/64 | 12532 | 5957 | 6658 | 9484 | 3459 | 2895 | 3601 | 688 |
| X/256 | 10780 | 9576 | 9573 | 2419 | 759 | 430 | 1175 | 632 |
| 0 | 7008 | 1493 | 727 | 1408 | 676 | 557 | 742 | 324 |
| D 10 | | | | | | | | |
| 1X | 20316 | 13497 | 12626 | 15684 | 13408 | 13550 | 9667 | 13437 |
| X/4 | 9454 | 8516 | 13572 | 4626 | 2175 | 7753 | 4056 | 2474 |
| X/16 | 4718 | 2203 | 4327 | 2009 | 5030 | 482 | 859 | 1052 |
| X/64 | 2819 | 2695 | 1044 | 954 | 503 | 885 | 311 | 927 |
| X/256 | 2792 | 1944 | 1402 | 405 | 1008 | 534 | 616 | #DIV/0! |
| 0 | 2009 | 648 | 301 | 339 | 624 | 488 | 581 | 301 |
| Expand Only D 2 | 3321 | 2529 | 1821 | 1596 | 1354 | 1122 | 828 | 386 |
| Expand Only D 6 | 8115 | 1922 | 967 | 615 | 580 | 585 | 632 | 413 |
| Expand Only D 10 | 2276 | 1301 | 607 | 440 | 1162 | 494 | 724 | 477 |
| Expand then Prime D 2 | | | | | | | | |
| 1X | 11751 | 7822 | 5259 | 4463 | 4632 | 3481 | 3789 | 1553 |
| X/4 | 6761 | 4604 | 3332 | 2684 | 3336 | 2494 | 1587 | 675 |
| X/16 | 6378 | 4530 | 2610 | 2218 | 2066 | 2032 | 1324 | 742 |
| X/64 | 5826 | 3719 | 2740 | 2145 | 1568 | 1509 | 929 | 576 |
| X/256 | 6328 | 4263 | 2487 | 1996 | 1389 | 1338 | 967 | 495 |
| 0 | 4283 | 2930 | 1955 | 1791 | 1525 | 1021 | 864 | 409 |
| Expand then Prime D 6 | | | | | | | | |
| 1X | 13945 | 3092 | 5678 | 575 | 779 | 1948 | 1263 | 323 |
| X/4 | 5666 | 3443 | 1986 | 579 | 1149 | 1739 | 1306 | 502 |
| X/16 | 10657 | 2798 | 3604 | 2292 | 993 | 1110 | 643 | 300 |
| X/64 | 7093 | 4319 | 351 | 935 | 1610 | 700 | 366 | 670 |
| X/256 | 7359 | 1376 | 89 | 775 | 459 | 1635 | 466 | 452 |
| 0 | 7601 | 2576 | 385 | 1165 | 465 | 1608 | 3263 | 488 |
| Expand then Prime D 10 | | | | | | | | |
| 1X | 3069 | 1703 | 1203 | 830 | 631 | 568 | 1376 | 451 |
| X/4 | 1716 | 1973 | 779 | 681 | 785 | 880 | 1123 | 383 |
| X/16 | 1196 | 1016 | 2104 | 793 | 1447 | 611 | 618 | 474 |
| X/64 | 2029 | 2196 | 526 | 549 | 497 | 479 | 438 | 325 |
| X/256 | 2387 | 974 | 596 | 469 | 609 | 510 | 367 | 291 |
| 0 | 3325 | 998 | 457 | 416 | 461 | 534 | 549 | 1564 |

TABLE 9
CD69 MFI

| | [18t15-12s on Y-axis where 1x = 250 nM | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 476 | 537 | 507 | 547 | 530 | 538 | 536 | 516 |
| X/4 | 495 | 542 | 572 | 571 | 568 | 575 | 567 | 553 |
| X/16 | 480 | 544 | 530 | 575 | 558 | 596 | 590 | 570 |
| X/64 | 495 | 532 | 567 | 555 | 597 | 575 | 575 | 551 |
| X/256 | 476 | 530 | 556 | 600 | 629 | 539 | 526 | 506 |
| 0 | 606 | 691 | 682 | 640 | 590 | 562 | 460 | 275 |
| D 6 | | | | | | | | |
| 1X | 279 | 252 | 243 | 250 | 258 | 288 | 298 | 257 |
| X/4 | 282 | 280 | 318 | 263 | 254 | 280 | 276 | 316 |
| X/16 | 266 | 248 | 295 | 276 | 303 | 330 | 325 | 274 |
| X/64 | 347 | 267 | 282 | 322 | 284 | 320 | 307 | 306 |
| X/256 | 294 | 297 | 266 | 341 | 315 | 317 | 315 | 283 |
| 0 | 338 | 308 | 283 | 327 | 358 | 312 | 312 | 278 |
| D 10 | | | | | | | | |
| 1X | 303 | 337 | 345 | 325 | 464 | 321 | 315 | 424 |
| X/4 | 273 | 300 | 247 | 358 | 353 | 346 | 346 | 319 |
| X/16 | 333 | 322 | 324 | 366 | 234 | 548 | 316 | 731 |
| X/64 | 346 | 293 | 347 | 395 | 264 | 353 | 381 | 402 |
| X/256 | 383 | 437 | 338 | 442 | 358 | 442 | 365 | 297 |
| 0 | 414 | 353 | 438 | 532 | 285 | 392 | 377 | 232 |
| Expand Only D 2 | 619 | 693 | 705 | 681 | 667 | 606 | 490 | 286 |
| Expand Only D 6 | 320 | 311 | 321 | 301 | 305 | 305 | 307 | 265 |
| Expand Only D 10 | 457 | 392 | 367 | 360 | 367 | 343 | 366 | 355 |
| Expand then Prime D 2 | | | | | | | | |
| 1X | 804 | 845 | 902 | 1002 | 963 | 950 | 877 | 707 |
| X/4 | 679 | 831 | 832 | 977 | 817 | 903 | 756 | 574 |
| X/16 | 634 | 764 | 885 | 809 | 856 | 736 | 684 | 524 |
| X/64 | 643 | 728 | 781 | 873 | 825 | 712 | 643 | 459 |
| X/256 | 674 | 697 | 765 | 835 | 735 | 749 | 615 | 408 |
| 0 | 759 | 790 | 853 | 851 | 801 | 717 | 549 | 346 |
| Expand then Prime D 6 | | | | | | | | |
| 1X | 304 | 270 | 314 | 582 | 367 | 305 | 473 | 217 |
| X/4 | 323 | 368 | 456 | 240 | 332 | 374 | 392 | 233 |
| X/16 | 289 | 277 | 297 | 280 | 267 | 313 | 239 | 446 |
| X/64 | 279 | 301 | 229 | 257 | 304 | 554 | 276 | 273 |
| X/256 | 428 | 348 | 261 | 351 | 230 | 309 | 259 | 232 |
| 0 | 343 | 276 | 3526 | 272 | 285 | 239 | 253 | 291 |
| Expand then Prime D 10 | | | | | | | | |
| 1X | 379 | 356 | 438 | 389 | 520 | 516 | 493 | 392 |
| X/4 | 459 | 413 | 403 | 429 | 368 | 451 | 555 | 454 |
| X/16 | 463 | 426 | 291 | 345 | 454 | 365 | 353 | 324 |
| X/64 | 322 | 361 | 335 | 462 | 386 | 420 | 372 | 537 |
| X/256 | 371 | 390 | 299 | 435 | 528 | 369 | 273 | 454 |
| 0 | 352 | 419 | 499 | 311 | 391 | 335 | 295 | 498 |

TABLE 10
CD16 MFI

| | [18t15-12s on Y-axis where 1x = 250 nM | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 321 | 370 | 415 | 427 | 398 | 386 | 394 | 409 |
| X/4 | 394 | 489 | 655 | 655 | 614 | 629 | 636 | 645 |
| X/16 | 423 | 516 | 672 | 782 | 745 | 856 | 827 | 789 |
| X/64 | 501 | 572 | 735 | 858 | 919 | 879 | 824 | 986 |
| X/256 | 424 | 538 | 853 | 992 | 895 | 1151 | 1099 | 1200 |
| 0 | 660 | 944 | 1344 | 1658 | 1921 | 1800 | 1630 | 1244 |
| D 6 | | | | | | | | |
| 1X | 359 | 374 | 332 | 365 | 333 | 395 | 318 | 361 |
| X/4 | 630 | 408 | 398 | 414 | 368 | 384 | 417 | 538 |
| X/16 | 448 | 357 | 396 | 338 | 352 | 415 | 45 | 406 |
| X/64 | 491 | 379 | 344 | 388 | 399 | 534 | 366 | 489 |
| X/256 | 411 | 510 | 40 | 414 | 379 | 469 | 397 | 414 |
| 0 | 555 | 508 | 355 | 587 | 573 | 462 | 754 | 392 |
| D 10 | | | | | | | | |
| 1X | 1210 | 939 | 1218 | 1502 | 984 | 1003 | 1094 | 1284 |
| X/4 | 849 | 794 | 778 | 989 | 822 | 851 | 870 | 980 |
| X/16 | 1046 | 863 | 600 | 500 | 518 | 1086 | 1114 | 813 |
| X/64 | 640 | 727 | 541 | 700 | 603 | 1559 | 372 | 464 |
| X/256 | 1857 | 953 | 556 | 1481 | 453 | 857 | 848 | 310 |
| 0 | 1111 | 716 | 661 | 1780 | 745 | 820 | 1349 | 626 |
| Expand Only D 2 | 561 | 896 | 1367 | 1594 | 1643 | 1730 | 1813 | 1389 |
| Expand Only D 6 | 600 | 468 | 474 | 537 | 594 | 731 | 590 | 392 |
| Expand Only D 10 | 995 | 1074 | 696 | 609 | 797 | 869 | 1235 | 538 |
| Expand then Prime D 2 | | | | | | | | |
| 1X | 297 | 370 | 369 | 364 | 356 | 409 | 503 | 804 |
| X/4 | 327 | 444 | 595 | 540 | 528 | 513 | 893 | 1281 |
| X/16 | 400 | 524 | 613 | 614 | 680 | 819 | 1264 | 1365 |
| X/64 | 477 | 448 | 693 | 587 | 684 | 995 | 1294 | 1430 |
| X/256 | 399 | 561 | 738 | 898 | 890 | 1013 | 1328 | 1427 |
| 0 | 430 | 753 | 1038 | 1231 | 1183 | 1333 | 1403 | 1042 |
| Expand then Prime D 6 | | | | | | | | |
| 1X | 459 | 469 | 40 | 408 | 351 | 338 | 401 | 274 |
| X/4 | 581 | 1144 | 432 | 346 | 561 | 388 | 49 | 306 |
| X/16 | 609 | 594 | 410 | 297 | 457 | 988 | 720 | 358 |
| X/64 | 738 | 731 | 332 | 642 | 343 | 536 | 359 | 376 |
| X/256 | 1124 | 343 | 510 | 405 | 339 | 626 | 542 | 295 |
| 0 | 915 | 393 | 595 | 495 | 570 | 557 | 672 | 326 |
| Expand then Prime D 10 | | | | | | | | |
| 1X | 547 | 500 | 607 | 417 | 920 | 542 | 483 | 724 |
| X/4 | 551 | 549 | 413 | 411 | 385 | 554 | 327 | 740 |
| X/16 | 722 | 761 | 481 | 434 | 604 | 496 | 584 | 580 |
| X/64 | 568 | 628 | 542 | 852 | 567 | 620 | 516 | 705 |
| X/256 | 561 | 587 | 361 | 370 | 549 | 706 | 476 | 972 |
| 0 | 609 | 617 | 550 | 550 | 453 | 497 | 645 | 411 |

TABLE 11
NKG2A MFI

| | [18t15-12s on Y-axis where 1x = 250 nM | | | | | | | |
|---|---|---|---|---|---|---|---|---|
| | 7t15-21s + ATF1 on X-axis where 1x = 200 nM and 100 nM respectively] | | | | | | | |
| Day and Condition | 1X | X/4 | X/16 | X/64 | X/256 | X/1024 | X/4096 | 0 |
| Prime & Expand | | | | | | | | |
| D 2 | | | | | | | | |
| 1X | 11751 | 12718 | 11661 | 13330 | 12213 | 13909 | 11838 | 12340 |
| X/4 | 14862 | 14508 | 17034 | 14809 | 15868 | 15888 | 16031 | 16498 |
| X/16 | 14901 | 16362 | 13864 | 14324 | 13653 | 17106 | 18319 | 16788 |
| X/64 | 15903 | 15985 | 16193 | 14433 | 16961 | 17513 | 16569 | 16929 |
| X/256 | 14610 | 15905 | 13190 | 17718 | 16448 | 14818 | 17682 | 16775 |
| 0 | 14449 | 12751 | 12559 | 11290 | 11648 | 12347 | 10902 | 4357 |
| D 6 | | | | | | | | |
| 1X | 2030 | 1503 | 1427 | 1316 | 1515 | 1337 | 1274 | 1719 |
| X/4 | 2273 | 1360 | 1069 | 1306 | 996 | 1626 | 1098 | 3581 |
| X/16 | 2584 | 1365 | 1404 | 641 | 503 | 729 | 700 | 2321 |
| X/64 | 3028 | 2371 | 632 | 655 | 1941 | 679 | 867 | 4363 |
| X/256 | 3273 | 2127 | 938 | 782 | 801 | 620 | 1237 | 682 |
| 0 | 10610 | 11660 | 2874 | 14942 | 9932 | 8254 | 14184 | 564 |
| D 10 | | | | | | | | |
| 1X | 2343 | 1836 | 2789 | 4810 | 2445 | 2138 | 2717 | 2619 |
| X/4 | 1575 | 3110 | 2412 | 3205 | 2519 | 1198 | 2981 | 3919 |
| X/16 | 2123 | 1850 | 1884 | 863 | 4175 | 1386 | 2873 | 945 |
| X/64 | 2392 | 1707 | 1628 | 2332 | 940 | 3568 | 615 | 637 |
| X/256 | 4654 | 2162 | 4051 | 1580 | 304 | 1138 | 1816 | 3875 |
| 0 | 10398 | 7902 | 8365 | 5698 | 14154 | 7920 | 12006 | 349 |
| Expand Only D 2 | 13797 | 13726 | 13137 | 12701 | 13094 | 13285 | 11974 | 4728 |
| Expand Only D 6 | 8285 | 10748 | 9688 | 11106 | 13553 | 13069 | 10831 | 638 |
| Expand Only D 10 | 16784 | 17322 | 10753 | 10540 | 14276 | 15293 | 12373 | 436 |
| Expand then Prime D 2 | | | | | | | | |
| 1X | 27375 | 29089 | 26745 | 28904 | 28795 | 25635 | 24010 | 6910 |
| X/4 | 31169 | 29938 | 26942 | 28101 | 30574 | 31331 | 18229 | 7141 |
| X/16 | 30305 | 28997 | 28178 | 28823 | 28677 | 26317 | 19423 | 8187 |
| X/64 | 28295 | 33287 | 31276 | 31204 | 25274 | 25542 | 24055 | 8412 |
| X/256 | 26385 | 28373 | 28018 | 28577 | 28384 | 27407 | 23038 | 8433 |
| 0 | 25332 | 25529 | 26586 | 22844 | 23209 | 20813 | 18779 | 4525 |
| Expand then Prime D 6 | | | | | | | | |
| 1X | 7242 | 14758 | 6611 | 13335 | 13310 | 10082 | 10734 | 371 |
| X/4 | 10041 | 12604 | 11162 | 24794 | 25093 | 18412 | 17599 | 502 |
| X/16 | 11236 | 11590 | 16165 | 9729 | 19520 | 15553 | 13407 | 372 |
| X/64 | 16584 | 18272 | 3970 | 19017 | 16939 | 26727 | 9495 | 441 |
| X/256 | 15137 | 13095 | 18351 | 24558 | 16073 | 25663 | 23470 | 528 |
| 0 | 14725 | 15676 | 18019 | 15387 | 13697 | 8101 | 15321 | 426 |
| Expand then Prime D 10 | | | | | | | | |
| 1X | 5198 | 7046 | 5437 | 2793 | 3969 | 3816 | 8820 | 412 |
| X/4 | 8335 | 4458 | 8546 | 6609 | 7670 | 10439 | 9293 | 399 |
| X/16 | 7827 | 5914 | 4777 | 8282 | 6845 | 10331 | 5791 | 344 |
| X/64 | 8139 | 8628 | 5329 | 10640 | 7261 | 10646 | 9005 | 708 |
| X/256 | 7078 | 9734 | 7079 | 9075 | 7129 | 10211 | 9284 | 544 |
| 0 | 9432 | 8288 | 2891 | 5111 | 2917 | 3674 | 7484 | 398 |

Alternatively, lymphoid progenitor cells, such as iPSC cells, or cord blood NK cells, may be cultured in a suitable media, and NK cells differentiated into a form that can be primed and/or expanded.

### Example 2. In Vitro Expand and Prime With Priming in the Middle or End

Material and Method: Purified NK Cells were treated with various concentrations of expansion agent 7t15-21s and ATF1 every 2 days. At Day 6 and Day 14, cells expanded with 200 nM and 100 nM of 7t15-21s and ATF1 were activated with 250 nM of 18t15-12s and the continued to be expanded with 200 nM and 100 nM of 7t15-21s and ATF1. After 6, 13 and 17 days NKs were then added at the indicated ratios to plate of K562-Luc2 cells (ATCC) in RPMI+10% heat-inactivated FBS. The plate was then incubated for 24 hours at 37 degrees C. and 5% CO2. Killing of the K562 cells was measured by luciferase readout. Lower EC:50 is considered better killing.

Result: This example demonstrates the in vitro activity and flow cytometry phenotype of NK cells generated by the above processes in large scale.

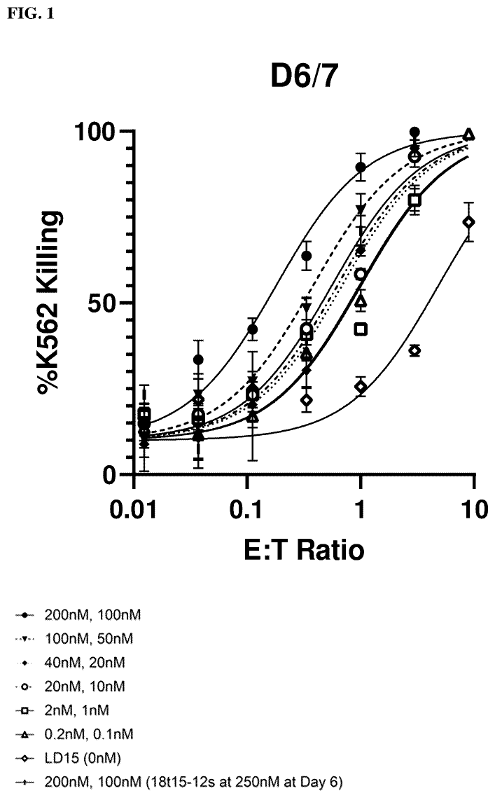

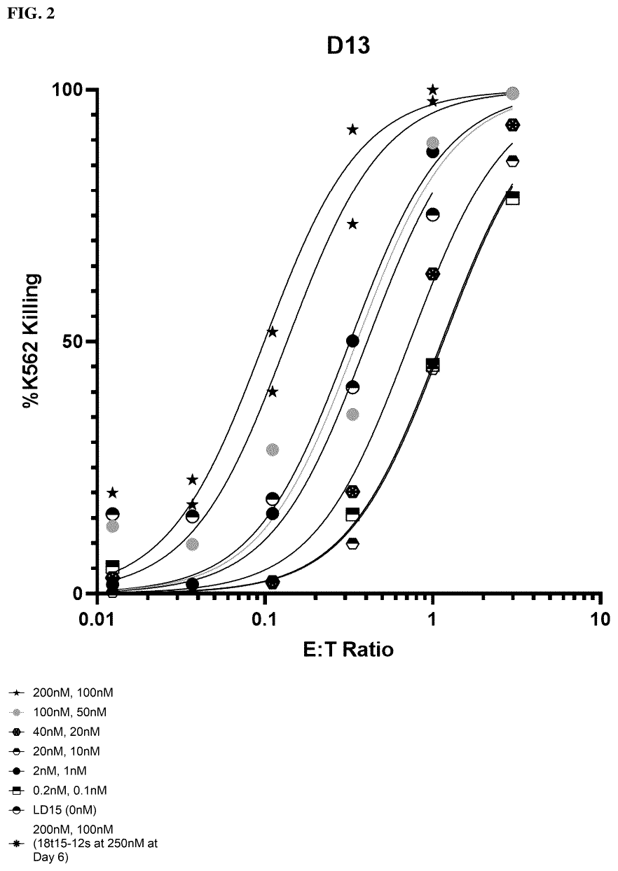

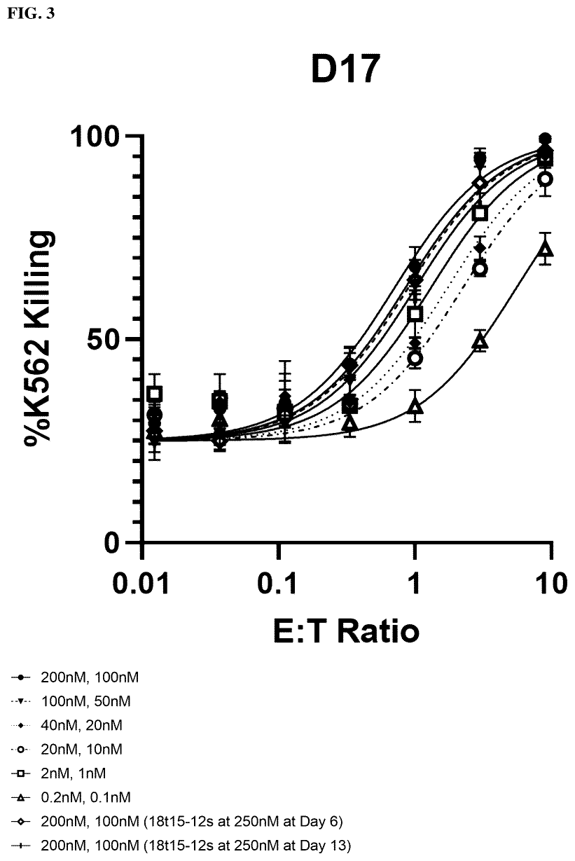

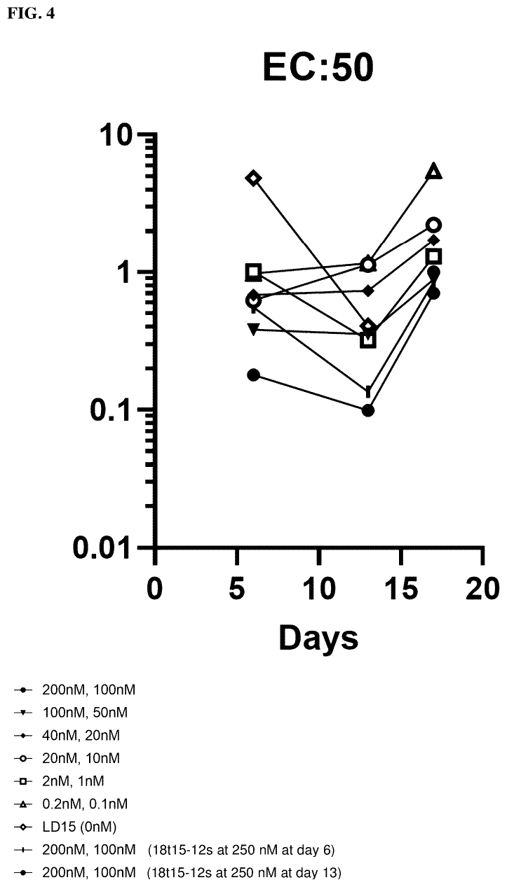

Results are shown in FIGS. 1-4.

### Example 3. In Vitro Culturing and Activity of Expand Only, Prime While Expand, Expand Then Prime, and Expand Then Prime & Expand

Material and Method: NK cells were isolated from whole blood using CD3 depletion and CD56 positive selection. NK cells selected were then cultured in 96-well plates in NK MACS media+supplements+10% HI-HAB, and were primed/expanded in the following conditions, where 1×7t15-21s and ATF1 is 200 nM and 100 nM respectively, and ×/4 is 50 nM and 25 nM respectively:
a) Expand Only: +1× or ×/4 7t15-21s and ATF1 for 4 days in 37deg, 5% CO2. At day 6 of culture and every 2 days following, 7t15-21s and ATF1 were replenished to 1× or ×/4 with fresh media.
b) Prime while Expand: +18t15-12s, 7t15-21s and ATF1 at 250 nM/1×, 250 nM/×/4, 62.5 nM/1×, or 62.5 nM/×/4 for 6 days in 37 deg, 5% CO2. At day 6 of culture and every 2 days following, 18t15-12s, 7t15-21s and ATF1 were replenished to the indicated concentrations with fresh media.
c) Expand then Prime: +1× or ×/4 7t15-21s and ATF1 for 4 days in 37 deg, 5% CO2. At day 6 of culture and every 2 days following, 7t15-21s and ATF1 were replenished to 1× or ×/4 with fresh media. At day 6 or day 14 as indicated, 18t15-12s was added at 250 nM or 62.5 nM for 3 hours.
d) Expand then Prime & Expand 3 hr: +1× or ×/4 7t15-21s and ATF1 for 4 days in 37 deg, 5% CO2. At day 6 of culture and every 2 days following, 7t15-21s and ATF1 were replenished to 1× or ×/4 with fresh media. At day 6 or day 14, 18t15-12s, 7t15-21s and ATF1 were added at 250 nM/1×, 250 nM/×/4, 62.5/1×, or 62.5/×/4 for 3 hours.
e) Expand then Prime & Expand 48 hr: +1× or ×/4 7t15-21s and ATF1 for 4 days in 37 deg, 5% CO2. At day 6 of culture and every 2 days following, 7t15-21s and ATF1 were replenished to 1× or ×/4 with fresh media. At day 6 or day 14, 18t15-12s, 7t15-21s and ATF1 were added at 250nM/1×, 250nM/×/4, 62.5nM/1×, or 62.5nM/×/4 for 48 hours.

To assess the phenotype of the NK cells generated by the above processes, at the appropriate timepoint, NK cells were harvested, washed, and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones were used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)

An Attune NXt flow cytometer was used. Data were then analyzed in Flowjo v10.7, gating on Live CD56+CD3-cells and assessing the median fluorescence intensity of each of the above-described markers. Increases in CD69, CD25, and NKG2A expression, and maintenance of CD16 expression, indicates a CIML-NK cell phenotype.

To assess killing activity of the NK cells generated by the above processes, at the appropriate timepoint, Cultured NK cells were harvested and washed, then resuspended in NK MACS Media with 10% human AB serum (Gibco)) and added to a 96-well plate with 10,000 Luciferase expressing K562 (K562-Luc) human tumor cells (ATCC) at the indicated effector to target (E:T) ratios 24-48 hours, with or without IL-2 (Miltenyi), after which luciferase activity (live K562 cells) was assessed by luciferase readout (Promega).

Result: This example demonstrates the in vitro activity and flow cytometry phenotype of NK cells generated by the above processes.

Results are shown in Tables 12-17, showing cumulative fold change in NK cell number, median fluorescence intensities for individual surface protein expression, and K562-Luc killing.

TABLE 12
Cumulative Fold Change

| | [18t15-12s], nM | | | | | | | | | |
|---|---|---|---|---|---|---|---|---|---|---|
| | | | | | | | Expand then | | Expand then | |
| | | Expand | Prime while | | Expand then | | Prime & | | Prime & | |
| | | Only | Expand | | Prime | | Expand 48 hr | | Expand 3 hr | |
| | | 0 | 250 | 62.5 | 250 | 62.5 | 250 | 62.5 | 250 | 62.5 |
| 6 days | | | | | | | | | | |
| Expansion | | | | | | | | | | |
| [7t15-21s + | 1X | 6 | 21 | 9 | 6 | 8 | 4 | 4 | 5 | 6 |
| ATF1], nM | X/4 | 5 | 22 | 11 | 5 | 6 | 3 | 3 | 4 | 5 |
| 14 days | | | | | | | | | | |
| Expansion | | | | | | | | | | |
| [7t15-21s + | 1X | 1212 | 5614 | 3555 | 1047 | 924 | 415 | 451 | 976 | 1393 |
| ATF1], nM | X/4 | 444 | 8553 | 4758 | 251 | 255 | 108 | 63 | 334 | 246 |

TABLE 13
CD16 MFI

| | [18t15-12s], nM | | | | | | | | | |
|---|---|---|---|---|---|---|---|---|---|---|
| | | | | | | | Expand then | | Expand then | |
| | | Expand | Prime while | | Expand then | | Prime & | | Prime & | |
| [7t15-21s + | | Only | Expand | | Prime | | Expand 48 hr | | Expand 3 hr | |
| ATF1], nM | Fresh | 0 nM | 250 | 62.5 | 250 | 62.5 | 250 | 62.5 | 250 | 62.5 |
| 0 days | 9927 | | | | | | | | | |
| Expansion | | | | | | | | | | |
| 6 days | | | | | | | | | | |
| Expansion | | | | | | | | | | |
| 1X | | 80 | −77 | 58 | 68 | 67 | 7 | 41 | 39 | 17 |
| X/4 | | 55 | −89 | 5 | 68 | 82 | −2 | 19 | 2 | −24 |
| 14 days | | | | | | | | | | |
| Expansion | | | | | | | | | | |
| 1X | | 444 | 133 | 162 | 352 | 275 | 166 | 251 | 239 | 167 |
| X/4 | | 459 | 142 | 198 | 227 | 195 | 224 | 215 | 142 | 181 |

TABLE 14
CD69 MFI

| | [18t15-12s], nM | | | | | | | | | |
|---|---|---|---|---|---|---|---|---|---|---|
| | | | | | | | Expand then | | Expand then | |
| | | Expand | Prime while | | Expand then | | Prime & | | Prime & | |
| [7t15-21s + | | Only | Expand | | Prime | | Expand 48 hr | | Expand 3 hr | |
| ATF1], nM | Fresh | 0 | 250 | 62.5 | 250 | 62.5 | 250 | 62.5 | 250 | 62.5 |
| 0 days | −12 | | | | | | | | | |
| Expansion | | | | | | | | | | |
| 6 days | | | | | | | | | | |
| Expansion | | | | | | | | | | |
| 1X | | 223 | 46 | 75 | 229 | 221 | 148 | 224 | 276 | 233 |
| X/4 | | 207 | 45 | 59 | 250 | 209 | 160 | 223 | 254 | 221 |
| 14 days | | | | | | | | | | |
| Expansion | | | | | | | | | | |
| 1X | | 270 | 77 | 98 | 346 | 328 | 229 | 439 | 325 | 244 |
| X/4 | | 250 | 90 | 99 | 446 | 231 | 319 | 271 | 397 | 265 |

TABLE 15
CD25 MFI

| | [18t15-12s], nM | | | | | | | | | |
|---|---|---|---|---|---|---|---|---|---|---|
| | | | | | | | Expand then | | Expand then | |
| | | Expand | Prime while | | Expand | | Prime & | | Prime & | |
| [7t15-21s + | | Only | Expand | | then Prime | | Expand 48 hr | | Expand 3 hr | |
| ATF1], nM | Fresh | 0 | 250 | 62.5 | 250 | 62.5 | 250 | 62.5 | 250 | 62.5 |
| 0 days | 250 | | | | | | | | | |
| Expansion | | | | | | | | | | |
| 6 days | | | | | | | | | | |
| Expansion | | | | | | | | | | |
| 1X | | 8857 | 72131 | 62798 | 10541 | 9487 | 21992 | 14248 | 11126 | 9852 |
| X/4 | | 5073 | 73126 | 63988 | 5700 | 5404 | 16999 | 8778 | 6274 | 4191 |
| 14 days | | | | | | | | | | |
| Expansion | | | | | | | | | | |
| 1X | | 3298 | 35856 | 22947 | 4847 | 3746 | 10383 | 7907 | 5401 | 4369 |
| X/4 | | 628 | 24446 | 11055 | 1455 | 894 | 778 | 878 | 1309 | 951 |

TABLE 16
NKG2A MFI

| | [18t15-12s], nM | | | | | | | | | |
|---|---|---|---|---|---|---|---|---|---|---|
| | | | | | | | Expand then | | Expand then | |
| | | Expand | Prime while | | Expand then | | Prime & | | Prime & | |
| [7t15-21s + | | Only | Expand | | Prime | | Expand 48 hr | | Expand 3 hr | |
| ATF1], nM | Fresh | 0 | 250 | 62.5 | 250 | 62.5 | 250 | 62.5 | 250 | 62.5 |
| 0 days | 649 | | | | | | | | | |
| Expansion | | | | | | | | | | |
| 6 days | | | | | | | | | | |
| Expansion | | | | | | | | | | |
| 1X | | 9025 | 162 | 1040 | 9930 | 9354 | 5874 | 6480 | 10404 | 9521 |
| X/4 | | 10770 | 118 | 267 | 13797 | 11287 | 7206 | 7945 | 11734 | 12256 |
| 14 days | | | | | | | | | | |
| Expansion | | | | | | | | | | |
| 1X | | 9021 | 106 | 476 | 11883 | 10805 | 4366 | 4886 | 9565 | 10588 |
| X/4 | | 9422 | 140 | 516 | 14945 | 10991 | 5321 | 5680 | 11689 | 9627 |

TABLE 17

| E:T of 1 (14 days of Expansion) | 48 hr K562 Luciferase Signal |
|---|---|
| E only (0.25X) | 20053 |
| E only (1X) | 12283 |
| E−>P last 3 hr (0.25X/0.25X) | 14844 |
| E−>P last 3 hr (0.25X/1X) | 10254 |
| E−>P last 3 hr (1X/0.25X) | 21180 |
| E−>P last 3 hr (1X/1X) | 13730 |
| E-->P&E last 1 cycle (0.25X/1X) | 14005 |
| E-->P&E last 1 cycle (1X/1X) | 15418 |
| E-->P&E last 1 cycle (0.25X/0.25X) | 29281 |
| E-->P&E last 1 cycle (1X/0.25X) | 23058 |
| E−>P&E last 3 hr (0.25X/0.25X) | 15858 |
| E−>P&E last 3 hr (0.25X/1X) | 12679 |
| E−>P&E last 3 hr (1X/0.25X) | 18787 |
| E−>P&E last 3 hr (1X/1X) | 12689 |
| P only (0.25X) | 12805 |
| P only (1X) | 12092 |
| P&E (0.25X/0.25X) | 12795 |
| P&E (0.25X/1X) | 14239 |
| P&E (1X/0.25X) | 10819 |
| P&E (1X/1X) | 11295 |

### Example 4. Large Scale in Vitro Culturing and Activity of Expand Only and Expand Then Prime

Material and Method: NK cells were isolated from a frozen leukopak on a MACS prodigy using CD3 depletion and CD56 positive selection. NK cells selected were then cultured in St. Gobain bags in NK MACS media+supplements+10% HI-HAB+25 nM 7t15-21s+50 nM ATF1 at an initial cellular concentration of 0.25e6/mL for 6 days in 37 deg, 5% CO2. At day 6 of culture and every 2 days following, cells were counted and diluted to a concentration of 0.25e6/mL and 7t15-21s and ATF1 were replenished to the appropriate concentration for the final media volume. At day 14, cells were either frozen (expand only) or cells were concentrated to 50e6/mL, and 18t15-12s was added to a final concentration of 250 nM (expand then prime). Cells thus primed were incubated at 37 deg, 5% CO2 for various times. After the indicated length of time (30 min, 1 h, 2 h, 3 h, 5 h or overnight) of 18t15-12s addition, cells were harvested, washed twice with HBSS (−/−), 0.5% HSA, and resuspended in freezing buffer (90% human serum, 10% DMSO). Cells were frozen at either 2e6 cells/mL or 20e6 cells/mL using a controlled rate freezer before transfer into the vapor phase of liquid nitrogen. Cells were then thawed, washed, counted, and utilized in downstream assays to measure function.

To assess the phenotype of the NK cells generated by the above processes, at the appropriate timepoint, NK cells were harvested, washed, and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones were used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)

An Attune NXt flow cytometer was used. Data were then analyzed in Flowjo v10.7, gating on Live CD56+CD3-cells and assessing the median fluorescence intensity of each of the above- described markers. Increases in CD69, CD25, and NKG2A expression, and maintenance of CD16 expression, indicates a CIML NK cell phenotype.

To assess killing activity of the NK cells generated by the above processes, at the appropriate timepoint, Cultured NK cells were harvested and washed, then resuspended in NK MACS Media with 10% human AB serum (Gibco)) and added to a 96-well plate with 10,000 Luciferase expressing K562 (K562-Luc) human tumor cells (ATCC) at the indicated effector to target (E:T) ratios 24-48 hours, with or without IL-2 (Miltenyi), after which luciferase activity (live K562 cells) was assessed by luciferase readout (Promega).

To assess the cytokine production capacity of NK cells generated by the above processes, NK cells were thawed, and then resuspended into NK MACS Media with 10% human AB serum (Gibco) and added to a 96-well plate with 10,000 Luciferase expressing K562 (K562-Luc) human tumor cells (ATCC) at effector to target (E:T) ratio of 1:1 for 24 hours or alone, after which supernatant was harvested and IFNg production assessed by IFNg ELISA (R&D Systems).

Result: This example demonstrates the in vitro activity and flow cytometry phenotype of NK cells generated by the above processes in large scale.

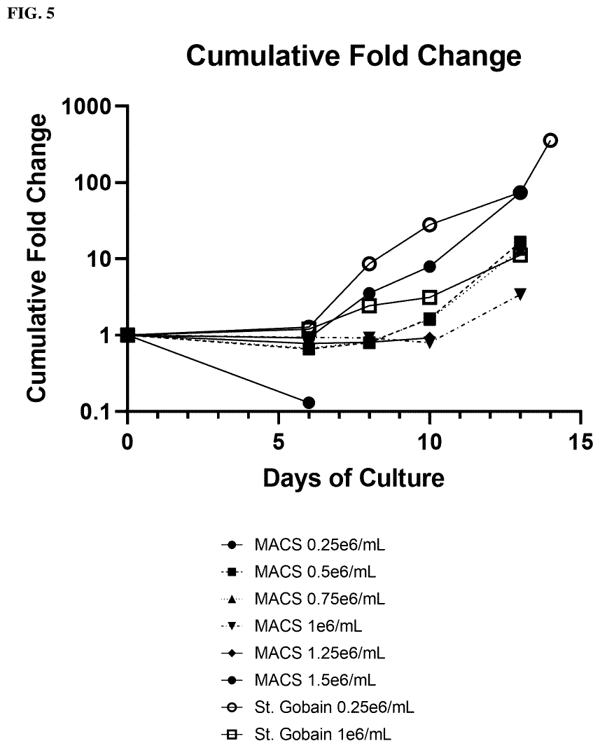

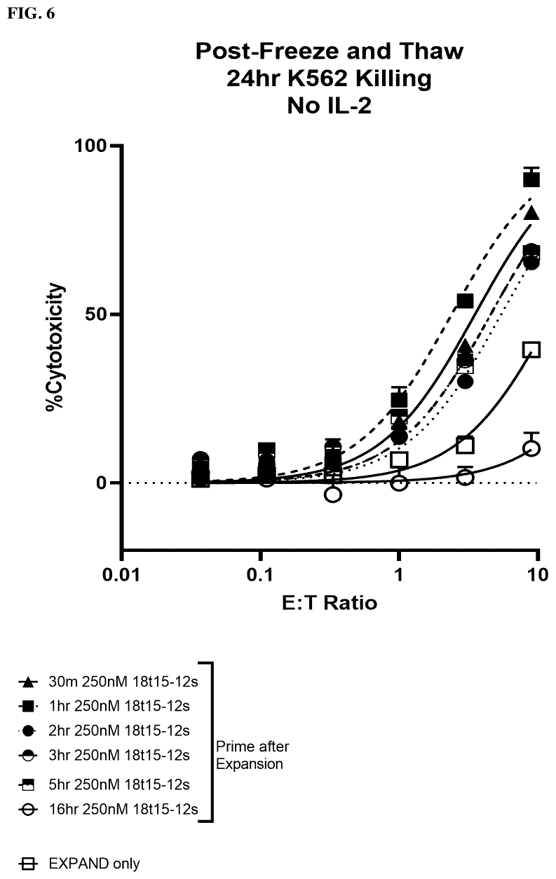

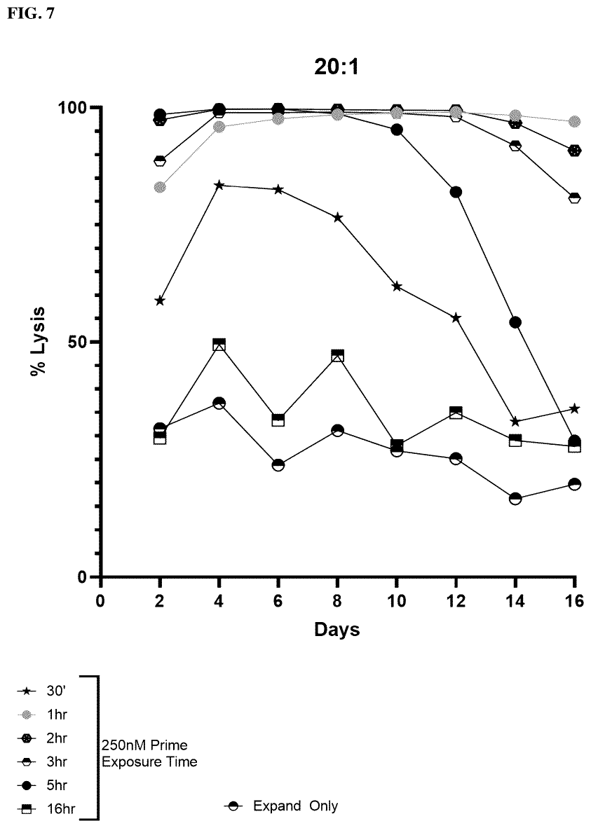

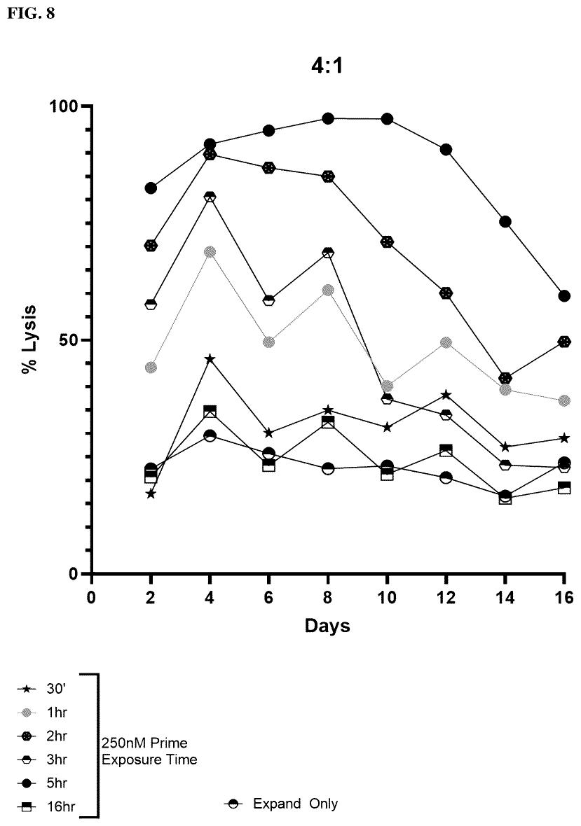

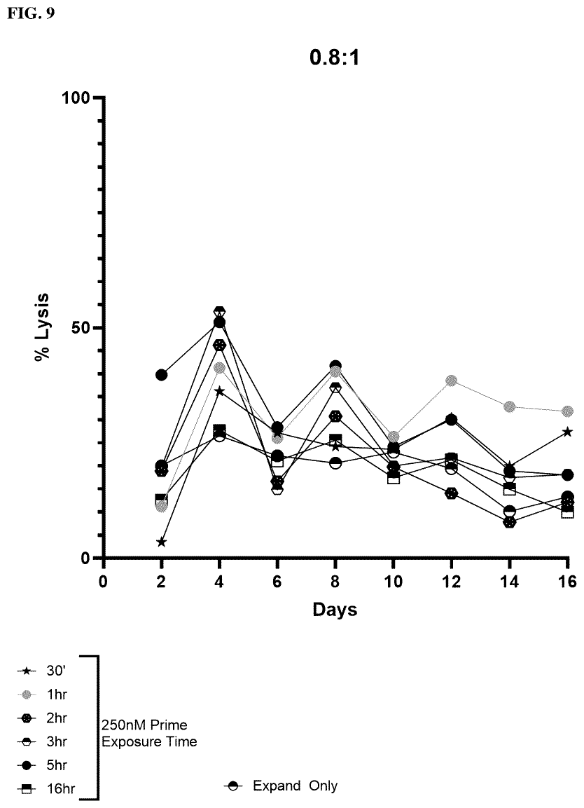

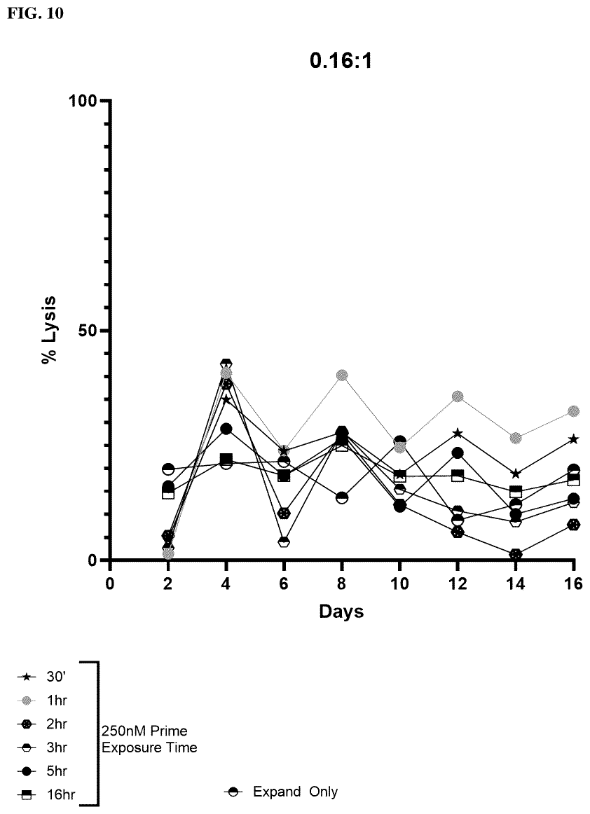

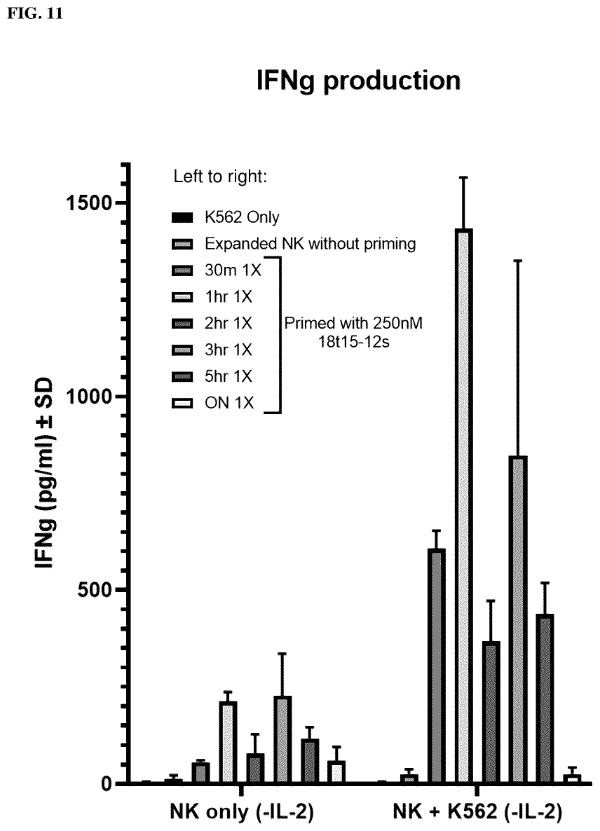

Results are shown in FIGS. 5-11.

### Example 5. Large Scale in Vitro Culturing and Activity of Expand Only, Prime Then Expand, and Expand Then Prime

Material and Method: NK cells were isolated from a frozen leukopak on a MACS prodigy using CD3 depletion and CD56 positive selection. NK cells selected were then cultured in St. Gobain bags in NK MACS media+supplements+10% HI-HAB+25 nM 7t15-21s+50 nM ATF1 at an initial cellular concentration of 0.25e6/mL for 6 days in 37 deg, 5% CO2. At day 6 of culture and every 2 days following, cells were counted and diluted to a concentration of 0.25e6/mL and 7t15-21s and ATF1 were replenished to the appropriate concentration for the final media volume. At day 14, cells were concentrated to various densities (2e6, 5e6, 10e6, 25e6, 35e6 or 50e6/mL), and 18t15-12s was added to a final concentration of 250 nM. Cells were incubated at 37 deg, 5%. After the indicated length of time (3 h or overnight) of 18t15-12s addition, cells were harvested, washed twice with HBSS (−/−), 0.5% HSA, and resuspended in freezing buffer (90% human serum, 10% DMSO). Cells were frozen at either 2e6 cells/mL or 20e6 cells/mL using a controlled rate freezer before transfer into the vapor phase of liquid nitrogen. Cells were then thawed, washed, counted, and utilized in downstream assays to measure function.

To assess the phenotype of the NK cells generated by the above processes, at the appropriate timepoint, NK cells were harvested, washed, and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones were used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)

An Attune NXt flow cytometer was used. Data were then analyzed in Flowjo v10.7, gating on Live CD56+CD3-cells and assessing the median fluorescence intensity of each of the above-described markers. Increases in CD69, CD25, and NKG2A expression, and maintenance of CD16 expression, indicates a CIML-NK cell phenotype.

To assess killing activity of the NK cells generated by the above processes, at the appropriate timepoint, Cultured NK cells were harvested and washed, then resuspended in NK MACS Media with 10% human AB serum (Gibco)) and added to a 96-well plate with 10,000 Luciferase expressing K562 (K562-Luc) human tumor cells (ATCC) at the indicated effector to target (E:T) ratios 24-48 hours, with or without IL-2 (Miltenyi), after which luciferase activity (live K562 cells) was assessed by luciferase readout (Promega).

To assess the cytokine production capacity of NK cells generated by the above processes, NK cells were thawed, and then resuspended into NK MACS Media with 10% human AB serum (Gibco) and added to a 96-well plate with 10,000 Luciferase expressing K562 (K562-Luc) human tumor cells (ATCC) at effector to target (E:T) ratio of 1:1 for 24 hours or alone, after which supernatant was harvested and IFNg production assessed by IFNg ELISA (R&D Systems).

To assess the in vivo persistence of NK cells generated by the above processes, NK cells were thawed and resuspended in HBSS at 20e6/mL. Between 2e6 and 5e6 cells (in, e.g., 100 uL) were injected into immunodeficient NSG mice (Jackson Laboratories, Bar Harbor Maine) intravenously. The mice were supported with dosing of human IL-2 (Miltenyi Biotec, 50,000 IU) every two days, and at day 7 blood was withdrawn and the number of NK cells were measured by staining with a flow panel consisting of:
Anti-CD56 (CMSSB clone),
Anti-CD3 (SK7 clone),
Live/Dead Yellow (Thermo Fisher),
anti-mouse CD45 (30-F11 clone), and
anti-human CD45 (HI30 clone) before fixation to lyse red blood cells. The cells were then analyzed on an Attune NXt flow cytometer for numbers of live huCD45+mouseCD45−CD3-cells.

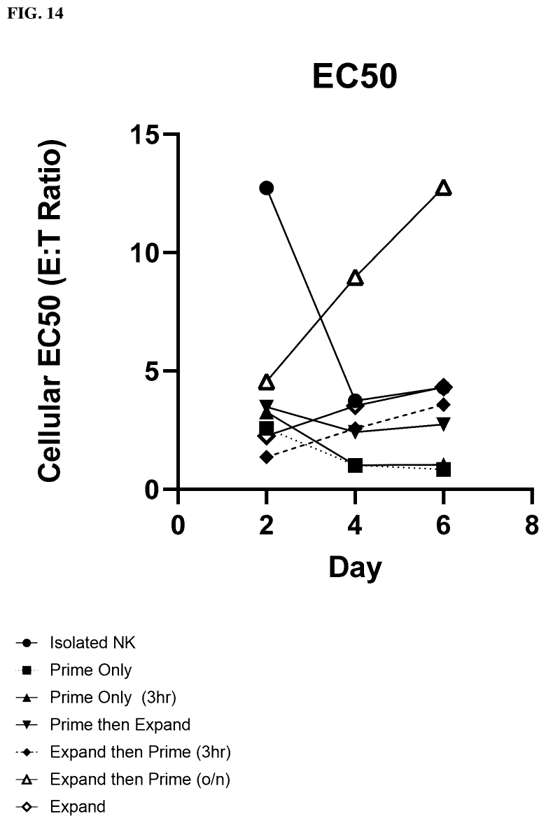

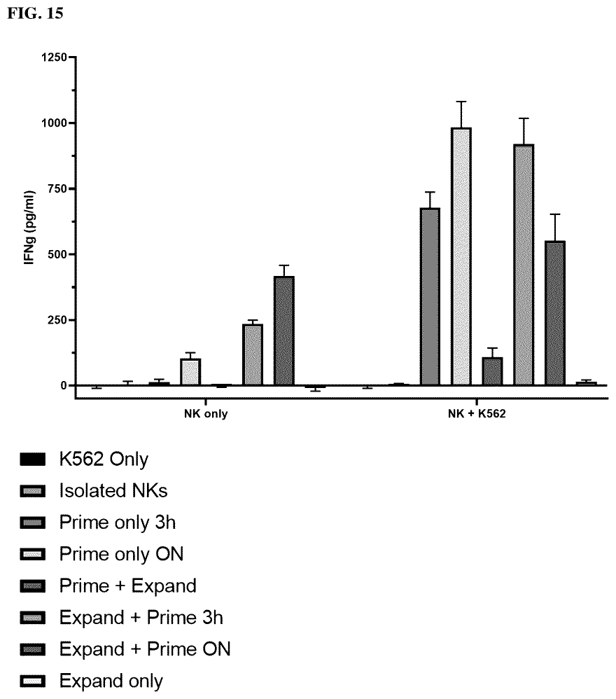

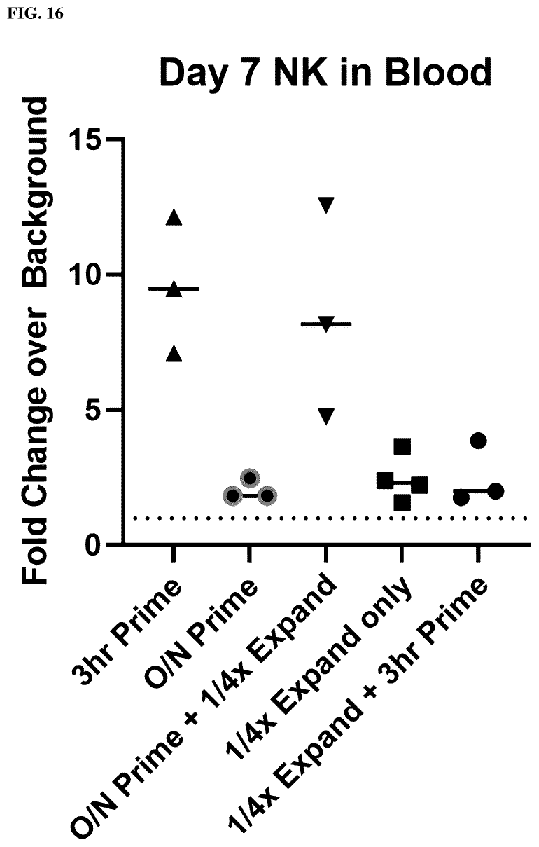

Result: This example demonstrates the in vitro activity and flow cytometry phenotype of NK cells generated by the above processes in large scale. Results are shown in FIGS. 12-16.

### Example 6. In Vitro Culturing and Activity of Expand Only and Expand Then Prime

Material and Method: NK cells were isolated from whole blood using CD3 depletion and CD56 positive selection. NK cells selected were then cultured in tissue culture treated flasks then transitioned to cell culture bags in NK MACS media+supplements+10% HI-HAB, and were expanded in the following conditions:
a) Expand Only: 50 nM 7t15-21s and 25 nM ATF1 for 4 days in 37 deg, 5% CO2. At day 5 of culture and every ⅔ days following, 7t15-21s and ATF1 were replenished to 50 nM and 25 nM respectively and cells were diluted to appropriate concentration with fresh media. At Day 14 cells were frozen in 90% HAB, 10% DMSO.
b) Expand then Prime: 50 nM 7t15-21s and 25 nM ATF1 for 4 days in 37 deg, 5% CO2. At day 5 of culture and every ⅔ days following, 7t15-21s and ATF1 were replenished to 50 nM and 25 nM respectively and cells were diluted to appropriate concentration with fresh media. At day 14, 18t15-12s was added at 250 nM for 3 hours. The cells were then frozen in 90% HAB, 10% DMSO.

To assess the phenotype of the NK cells generated by the above processes, frozen cells were thawed and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones were used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)

An Attune NXt flow cytometer was used. Data were then analyzed in Flowjo v10.7, gating on Live CD56+CD3-cells and assessing the median fluorescence intensity of each of the above-described markers. Increases in CD69, CD25, and NKG2A expression, and maintenance of CD16 expression, indicates a CIML-NK cell phenotype. Results are shown below in Tables 18-22.

TABLE 18.1
CD16 MFI

| Sample | Expand Only | Expand then Prime |
|---|---|---|
| Donor 1, Run 1 | 58.8 | 56 |
| Donor 1, Run 2 | 61.1 | 69.5 |
| Donor 2, Run 1 | 144 | 113 |
| Donor 2, Run 2 | 69.8 | 69.4 |
| Donor 3, Run 1 | 69.7 | 69.2 |
| Donor 3, Run 2 | 66.9 | 68.7 |
| Donor 4, Run 1 | 112 | 70.7 |
| Donor 4, Run 2 | 68.3 | 67.8 |
| Donor 4, Run 3 | 75.6 | 74.1 |
| Donor 5, Run 1 | 170 | 187 |
| Donor 5, Run 2 | 94.5 | 90.5 |
| Donor 5, Run 3 | 78.2 | 69.7 |
| Donor 6, Run 1 | 74.5 | 74.8 |
| Donor 6, Run 2 | 64.5 | 60.1 |
| Donor 6, Run 3 | 57.9 | 60.1 |
| Donor 8, Run 1 | 97.4 | 79 |
| Donor 8, Run 2 | 68.3 | 67.3 |
| Donor 8, Run 3 | 54.6 | 60.9 |
| Donor 10, Run 1 | 86.2 | 75.4 |
| Donor 10, Run 2 | 80.7 | 83.5 |
| Donor 10, Run 3 | 73.7 | 70.6 |
| Donor 11, Run 1 | 113 | 126 |
| Donor 11, Run 2 | 77 | 70 |
| Donor 11, Run 3 | 63.3 | 55 |
| Donor 12, Run 1 | 63.1 | 61.9 |
| Donor 12, Run 2 | 59 | 58.7 |
| Donor 12, Run 3 | 60.7 | 62.7 |
| Donor 15, Run 1 | 56.8 | 55.2 |
| Donor 15, Run 2 | 60.1 | 54.6 |
| Donor 15, Run 3 | 57.9 | 55.7 |
| Donor 16, Run 1 | 77 | 79 |
| Donor 16, Run 2 | 98.1 | 82.4 |
| Donor 16, Run 3 | 125 | 113 |
| Donor 17, Run 1 | 63 | 63 |
| Donor 17, Run 2 | 116 | 92.2 |
| Donor 17, Run 3 | 85 | 83.2 |
| Donor 18 | 97.4 | 72.5 |
| Donor 20, Run 1 | 60.8 | 57.8 |
| Donor 20, Run 2 | 58.5 | 56.5 |
| Donor 20, Run 3 | 84.1 | 76.5 |
| Donor 22, Run 1 | 56.5 | 49.3 |
| Donor 22, Run 2 | 66.9 | 50.8 |
| Donor 22, Run 3 | 64 | 59 |
| Donor 23, Run 1 | 55.4 | 52.3 |
| Donor 23, Run 2 | 51.3 | 52.5 |
| Donor 23, Run 3 | 54.1 | 55.7 |

TABLE 19
CD69 MFI

| Sample | Expand Only | Expand then Prime |
|---|---|---|
| Donor 1, Run 1 | 129 | 141 |
| Donor 1, Run 2 | 201 | 262 |
| Donor 2, Run 1 | 336 | 347 |
| Donor 2, Run 2 | 102 | 114 |
| Donor 3, Run 1 | 404 | 531 |
| Donor 3, Run 2 | 163 | 250 |
| Donor 4, Run 1 | 275 | 266 |
| Donor 4, Run 2 | 221 | 242 |
| Donor 4, Run 3 | 125 | 141 |
| Donor 5, Run 1 | 148 | 221 |
| Donor 5, Run 2 | 135 | 246 |
| Donor 5, Run 3 | 0.86 | 12.7 |
| Donor 6, Run 1 | 138 | 148 |
| Donor 6, Run 2 | 63.8 | 90.4 |
| Donor 6, Run 3 | 74.3 | 96.4 |
| Donor 8, Run 1 | 135 | 137 |
| Donor 8, Run 2 | 237 | 379 |
| Donor 8, Run 3 | 219 | 149 |
| Donor 10, Run 1 | 162 | 201 |
| Donor 10, Run 2 | 157 | 266 |
| Donor 10, Run 3 | 105 | 126 |
| Donor 11, Run 1 | 247 | 276 |
| Donor 11, Run 2 | 108 | 117 |
| Donor 11, Run 3 | 111 | 125 |
| Donor 12, Run 1 | 94.5 | 118 |
| Donor 12, Run 2 | −4.09 | 2.8 |
| Donor 12, Run 3 | 94.5 | 113 |
| Donor 15, Run 1 | 97.9 | 126 |
| Donor 15, Run 2 | 104 | 122 |
| Donor 15, Run 3 | 138 | 174 |
| Donor 16, Run 1 | 200 | 259 |
| Donor 16, Run 2 | 248 | 267 |
| Donor 16, Run 3 | 118 | 157 |
| Donor 17, Run 1 | 86.9 | 90.8 |
| Donor 17, Run 2 | 137 | 146 |
| Donor 17, Run 3 | 128 | 154 |
| Donor 18 | 214 | 315 |
| Donor 20, Run 1 | 90 | 93.7 |
| Donor 20, Run 2 | 97 | 101 |
| Donor 20, Run 3 | 101 | 110 |
| Donor 22, Run 1 | 50.8 | 115 |
| Donor 22, Run 2 | 32.7 | 137 |
| Donor 22, Run 3 | 61.9 | 92.4 |
| Donor 23, Run 1 | 106 | 113 |
| Donor 23, Run 2 | 115 | 119 |
| Donor 23, Run 3 | 97.9 | 90.4 |

TABLE 20
CD25 MFI

| Sample | Expand Only | Expand then Prime |
|---|---|---|
| Donor 1, Run 1 | 122 | 144 |
| Donor 1, Run 2 | 374 | 450 |
| Donor 2, Run 1 | 186 | 212 |
| Donor 2, Run 2 | 15.2 | 18.2 |
| Donor 3, Run 1 | 41.4 | 96.4 |
| Donor 3, Run 2 | −14.6 | −6.54 |
| Donor 4, Run 1 | 403 | 270 |
| Donor 4, Run 2 | 286 | 295 |
| Donor 4, Run 3 | 59.8 | 68.6 |
| Donor 5, Run 1 | 74.9 | 81.1 |
| Donor 5, Run 2 | 15.8 | 23.3 |
| Donor 5, Run 3 | 40.6 | 40.7 |
| Donor 6, Run 1 | 62.2 | 81.6 |
| Donor 6, Run 2 | 78.1 | 101 |
| Donor 6, Run 3 | 60.1 | 96.3 |
| Donor 8, Run 1 | 80.7 | 75.3 |
| Donor 8, Run 2 | 69.4 | 132 |
| Donor 8, Run 3 | 162 | 116 |
| Donor 10, Run 1 | 58.2 | 52.6 |
| Donor 10, Run 2 | 27.6 | 44 |
| Donor 10, Run 3 | 54.9 | 60.8 |
| Donor 11, Run 1 | 431 | 451 |
| Donor 11, Run 2 | 5.95 | 10.1 |
| Donor 11, Run 3 | 33.1 | 18.8 |
| Donor 12, Run 1 | 25.1 | 28.2 |
| Donor 12, Run 2 | 86.3 | 86.3 |
| Donor 12, Run 3 | 86.8 | 112 |
| Donor 15, Run 1 | −3.58 | 3.58 |
| Donor 15, Run 2 | 10.7 | 8.96 |
| Donor 15, Run 3 | −8.06 | −11.6 |
| Donor 16, Run 1 | 315 | 289 |
| Donor 16, Run 2 | 257 | 237 |
| Donor 16, Run 3 | 69.2 | 106 |
| Donor 17, Run 1 | 21.8 | 58.5 |
| Donor 17, Run 2 | 423 | 410 |
| Donor 17, Run 3 | 108 | 126 |
| Donor 18 | 96.6 | 137 |
| Donor 20, Run 1 | 47.2 | 55.1 |
| Donor 20, Run 2 | 56 | 54.7 |
| Donor 20, Run 3 | 64.2 | 71.5 |
| Donor 22, Run 1 | 89.2 | 109 |
| Donor 22, Run 2 | 59.9 | 67.6 |
| Donor 22, Run 3 | 43 | 41.2 |
| Donor 23, Run 1 | 26.9 | 44.4 |
| Donor 23, Run 2 | 47.9 | 42.4 |
| Donor 23, Run 3 | 34.9 | 34 |

TABLE 21
NKG2A MFI

| Sample | Expand Only | Expand then Prime |
|---|---|---|
| Donor 1, Run 1 | 2091 | 2268 |
| Donor 1, Run 2 | 5200 | 5890 |
| Donor 2, Run 1 | 8143 | 6493 |
| Donor 2, Run 2 | 2329 | 2087 |
| Donor 3, Run 1 | 6972 | 6787 |
| Donor 3, Run 2 | 2898 | 3031 |
| Donor 4, Run 1 | 10244 | 6832 |
| Donor 4, Run 2 | 5235 | 5011 |
| Donor 4, Run 3 | 2858 | 2593 |
| Donor 5, Run 1 | 4833 | 5869 |
| Donor 5, Run 2 | 5623 | 5572 |
| Donor 5, Run 3 | 4368 | 3900 |
| Donor 6, Run 1 | 6259 | 6673 |
| Donor 6, Run 2 | 4841 | 5340 |
| Donor 6, Run 3 | 4017 | 5270 |
| Donor 8, Run 1 | 2863 | 2879 |
| Donor 8, Run 2 | 6175 | 6809 |
| Donor 8, Run 3 | 5148 | 5360 |
| Donor 10, Run 1 | 5734 | 5045 |
| Donor 10, Run 2 | 5062 | 5714 |
| Donor 10, Run 3 | 3896 | 4149 |
| Donor 11, Run 1 | 7434 | 7384 |
| Donor 11, Run 2 | 2664 | 2354 |
| Donor 11, Run 3 | 2075 | 2114 |
| Donor 12, Run 1 | 3162 | 3298 |
| Donor 12, Run 2 | 4716 | 4516 |
| Donor 12, Run 3 | 2504 | 3031 |
| Donor 15, Run 1 | 5214 | 4971 |
| Donor 15, Run 2 | 5573 | 5132 |
| Donor 15, Run 3 | 5412 | 4641 |
| Donor 16, Run 1 | 3890 | 4674 |
| Donor 16, Run 2 | 4781 | 4644 |
| Donor 16, Run 3 | 2546 | 2498 |
| Donor 17, Run 1 | 1237 | 1168 |
| Donor 17, Run 2 | 3802 | 3056 |
| Donor 17, Run 3 | 2567 | 2809 |
| Donor 18 | 5202 | 4270 |
| Donor 20, Run 1 | 2642 | 2366 |
| Donor 20, Run 2 | 2622 | 2509 |
| Donor 20, Run 3 | 2033 | 2431 |
| Donor 22, Run 1 | 6972 | 7115 |
| Donor 22, Run 2 | 6972 | 6902 |
| Donor 22, Run 3 | 3944 | 3763 |
| Donor 23, Run 1 | 2984 | 3252 |
| Donor 23, Run 2 | 2493 | 2275 |
| Donor 23, Run 3 | 4497 | 4740 |

TABLE 22
Cell Size: FSC MFI

| Sample | Expand Only | Expand then Prime |
|---|---|---|
| Donor 1, Run 1 | 377600 | 392960 |
| Donor 1, Run 2 | 337664 | 340224 |
| Donor 2, Run 1 | 346112 | 344576 |
| Donor 2, Run 2 | 374272 | 372992 |
| Donor 3, Run 1 | 382976 | 379136 |
| Donor 3, Run 2 | 356608 | 363264 |
| Donor 4, Run 1 | 376320 | 361728 |
| Donor 4, Run 2 | 336896 | 331008 |
| Donor 4, Run 3 | 383488 | 387072 |
| Donor 5, Run 1 | 348928 | 363776 |
| Donor 5, Run 2 | 322816 | 325120 |
| Donor 5, Run 3 | 412000 | 422000 |
| Donor 6, Run 1 | 399360 | 403000 |
| Donor 6, Run 2 | 421000 | 419000 |
| Donor 6, Run 3 | 380160 | 420000 |
| Donor 8, Run 1 | 358912 | 351744 |
| Donor 8, Run 2 | 354048 | 349184 |
| Donor 8, Run 3 | 321280 | 319232 |
| Donor 10, Run 1 | 417000 | 420000 |
| Donor 10, Run 2 | 422000 | 432000 |
| Donor 10, Run 3 | 367360 | 387584 |
| Donor 11, Run 1 | 395008 | 395520 |
| Donor 11, Run 2 | 377344 | 370944 |
| Donor 11, Run 3 | 368384 | 365824 |
| Donor 12, Run 1 | 382720 | 387840 |
| Donor 12, Run 2 | 434000 | 430000 |
| Donor 12, Run 3 | 414000 | 415000 |
| Donor 15, Run 1 | 437000 | 431000 |
| Donor 15, Run 2 | 439000 | 418000 |
| Donor 15, Run 3 | 486000 | 475000 |
| Donor 16, Run 1 | 347136 | 335616 |
| Donor 16, Run 2 | 344320 | 341248 |
| Donor 16, Run 3 | 381440 | 384768 |
| Donor 17, Run 1 | 337395 | 337782 |
| Donor 17, Run 2 | 359936 | 354560 |
| Donor 17, Run 3 | 384256 | 400000 |
| Donor 18 | 335360 | 325888 |
| Donor 20, Run 1 | 347354 | 341053 |
| Donor 20, Run 2 | 356595 | 349220 |
| Donor 20, Run 3 | 357376 | 359168 |
| Donor 22, Run 1 | 360448 | 362752 |
| Donor 22, Run 2 | 350720 | 346880 |
| Donor 22, Run 3 | 415000 | 404000 |
| Donor 23, Run 1 | 350464 | 364299 |
| Donor 23, Run 2 | 360704 | 355840 |
| Donor 23, Run 3 | 451000 | 456000 |

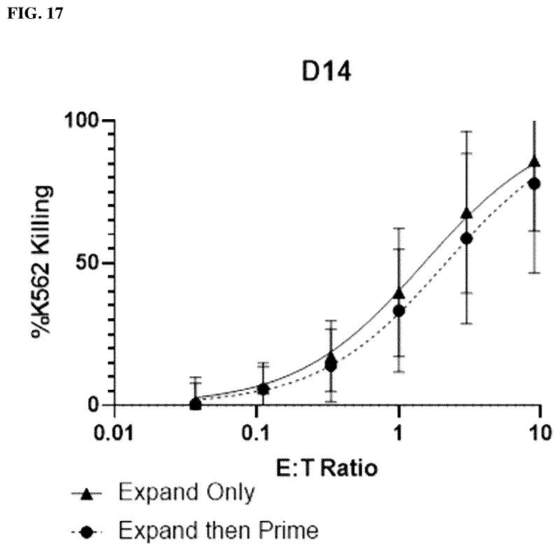

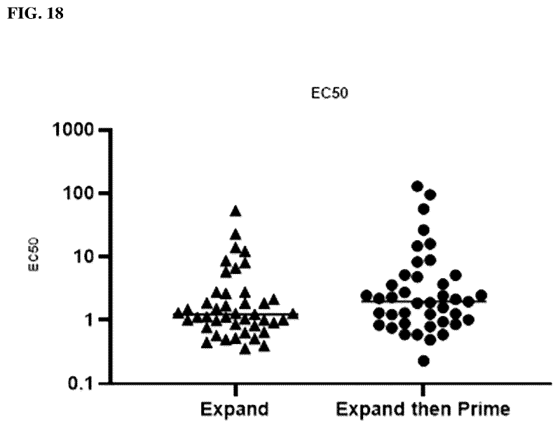

To assess killing activity of the NK cells generated by the above processes, at the appropriate timepoint, Cultured NK cells were harvested and washed, then resuspended in NK MACS Media with 10% human AB serum (Gibco)) and added to a 96-well plate with 10,000 Luciferase expressing K562 (K562-Luc) human tumor cells (ATCC) at the indicated effector to target (E:T) ratios 24-48 hours, with or without IL-2 (Miltenyi), after which luciferase activity (live K562 cells) was assessed by luciferase readout (Promega). Results are shown in FIGS. 17-18.

Result: This example demonstrates the in vitro activity and flow cytometry phenotype of NK cells generated by the above processes.

### Example 7. In Vivo Killing Activity of CIML-NK Cells

To assess killing efficacy in vivo, NSG mice are implanted with K562-Luc (ATCC) tumor cells. At the end of the NK cell culture, cells are harvested, washed, and 2-10e6NK cells are injected intravenously into tumor bearing animals, with some control mice left uninjected. The mice are supported with q2d dosing of human IL-2 (50,000 IU), and tumor growth is measured weekly by injecting mice with luciferin and reading luciferase on a capable instrument.

### Example 8. Clinical Trial Protocols

NK cells as disclosed above may be thawed, if cryopreserved, and infused into patients in a suitable medium, for the treatment of diseases such as cancers. Exemplary methods of testing for the safety and efficacy of NK cells in, e.g., acute myeloid leukemia and myelodysplastic syndrome, are disclosed in clinical trial protocol no.s NCT04354025, NCT03068819, NCT01898793, NCT02782546 and NCT04893915. These protocols involve memory NK cells which have been primed using either a cocktail of IL-12, IL-15, and IL-18 , or a priming fusion protein complex, then optionally expanded. Similar clinical trials may be run using memory NK cells which have been expanded then primed, or expanded and primed concurrently.

TABLE 23
CTP NCT04893915

| | |
|---|---|
| Study No. | NCT04893915 |
| Title | A Phase 2 Study of WU-NK-101 in Relapsed/Refractory AML and MDS |
| Summary/ | This is a phase 2 study with a lead-in cohort of WU-NK-101, a cytokine- |
| Rationale | induced memory-like NK cell product derived from leukapheresed |
| | allogeneic donor NK cells activated ex vivo using HCW-9201, a GMP- |
| | grade fusion cytokine comprising IL-12, IL-15, and IL-18. Patients with |
| | relapsed/refractory acute myeloid leukemia (AML) or myelodysplastic |
| | syndrome (MDS) will receive lymphodepleting chemotherapy (Flu/Cy) |
| | and two infusions of WU-NK-101 at the previously defined maximum |
| | tolerated dose (MTD), fourteen days apart. Low dose rhIL-2 will be |
| | administered to patients for in vivo expansion following cell infusion. |
| | Patients will be assessed for anti-leukemic efficacy and safety. Re- |
| | infusion of patients who relapsed after clinical response will be |
| | considered. |
| Conditions | Relapsed Acute Myeloid Leukemia |
| | Refractory Acute Myeloid Leukemia |
| | Myelodysplastic Syndromes |
| Study | Allocation: Non-Randomized |
| Design | Intervention Model: Sequential Assignment |
| | Masking: None (Open Label) |
| | Primary Purpose: Treatment |
| Interventions | Biological: WU-NK-01 - Cell product processing is performed at the |
| | Siteman Cancer Center Biological Therapy Core or another FACT- |
| | accredited cellular therapy production facility that can manufacture WU- |
| | NK-101 per the IND CMC. |
| | Drugs: Fludarabine. Cyclophosphamide (lymphodepleting regimen); IL- |
| | 2 will start approximately 2-4 hours after the WU-NK-101 infusions. |
| | Procedures: Leukapheresis, peripheral blood for correlative studies, bone |
| | marrow for correlative studies |
| Schedule | Experimental: Lead In Cohort Recipient: WU-NK-101 |
| | Fludarabine and cyclophosphamide beginning on Day −6. |
| | NK cell product will be infused on Day 0. |
| | IL-2 will begin 2-4 hours after infusion and will continue every other day |
| | through Day 12 for a total of 7 doses. |
| | NK cell product will be infused into the recipient on Day +14. |
| | IL-2 will begin 2-4 hours after infusion and will continue every other day |
| | through Day 26 for an additional 7 doses, and a total of 14 doses, to a |
| | maximum of two vials of rhIL-2 per IL-2 course. |
| | In the Lead-in Cohort, three patients will receive WU-NK-101 on Day 0 |
| | and Day +14, receiving the maximum NK cells generated, capped at |
| | 20 × 10\hat{ }6/kg. |
| | Patients that have an initial response to WU-NK-101 but then |
| | subsequently relapse or progress will be able to receive a third dose of |
| | WU-NK-101 with or without lymphodepleting chemotherapy depending |
| | on the interval duration between the second dose and relapse, after |
| | approval by the study PI. The third dose should be administered not less |
| | than 45 days from Day 0. |
| | Experimental: Phase II Recipient: WU-NK-01 |
| | Fludarabine and cyclophosphamide beginning on Day −6. |
| | NK cell product will be infused on Day 0. |
| | IL-2 will begin 2-4 hours after infusion and will continue every other day |
| | through Day 12 for a total of 7 doses. |
| | NK cell product will be infused into the recipient on Day +14. |
| | IL-2 will begin 2-4 hours after infusion and will continue every other day |
| | through Day 26 for an additional 7 doses, and a total of 14 doses, to a |
| | maximum of two vials of rhIL-2 per IL-2 course. |
| | Will receive WU-NK-101 on Day 0 and Day +14, receiving the |
| | maximum NK cells generated, capped at 20 × 10\hat{ }6/kg. |
| | Patients that have an initial response to WU-NK-101 but then |
| | subsequently relapse or progress will be able to receive a third dose of |
| | WU-NK-101 with or without lymphodepleting chemotherapy depending |
| | on the interval duration between the second dose and relapse, after |
| | approval by the study PI. The third dose should be administered not less |
| | than 45 days from Day 0. |
| | Experimental: Donor |
| | The allogeneic donor will undergo non-mobilized large volume (20-L) |
| | leukapheresis on Day −1. |
| | On Day +13 the allogeneic donor will again undergo non-mobilized large |
| | volume (20-L) leukapheresis |
| Primary | Overall response rate (ORR) of recipients [Time Frame: Through 12- |
| Outcome | month follow-up], defined as the proportion of patients achieving |
| Measures | complete remission (CR), complete remission with partial hematologic |
| | recovery (CRh), and complete remission with incomplete blood count |
| | recovery (CRi). Response will be assessed according to the criteria from |
| | the International Working Group Response Criteria. |
| Secondary | Overall survival (OS) of recipients [Time Frame: Through completion |
| Outcome | of follow-up (estimated to be 12 months)], defined as time from first |
| Measures | dose of lymphodepleting chemotherapy (LDC) until death from any |
| | cause. |
| | Event free survival (EFS) of recipients [Time Frame: Through |
| | completion of follow-up (estimated to be 12 months)], defined as time |
| | from first dose of lymphodepleting chemotherapy (LDC) until treatment |
| | failure, relapse from complete response, or death |
| | Duration of overall response (DOR) of recipients [Time Frame: |
| | Through 12-month follow-up], defined as duration for first occurrence of |
| | documented ORR until disease progression or death. |
| | Duration of complete response (DoCR) of recipients [Time Frame: |
| | Through 12-month follow-up], defined as duration from documented |
| | complete remission until disease progression or death |
| | Proportion of recipients that receive multiple doses of WU-NK-101 |
| | [Time Frame: Through Day +14 of all recipients enrolled (estimated to |
| | be 19 months)], |
| | Number of dose-limiting toxicities (DLTs) that recipients experience in |
| | the safety lead-in cohort [Time Frame: Through Day 28] |
| | Mortality rate of recipients [Time Frame: Day +30; Time Frame: Day +100] |
| | Number of adverse events experienced by recipients [Time Frame: |
| | Through Day +100]. |
| | Incidence, nature, and severity of adverse events |
| | tracked. Adverse events collected from Day 0 to Day +35; however, |
| | bone marrow suppression (ANC < 500/μL) and adverse events of graft- |
| | versus-host disease (GVHD) involving the liver, skin, or gastrointestinal |
| | tract recorded until Day +100. |
| | Proportion of recipients with prolonged cytopenia [Time Frame: At 8 |
| | weeks] |
| | Change in quality of life experienced by recipients as measured by the |
| | European Organization for Research and Treatment of Cancer Quality- |
| | of-Life Questionnaire (EORTC QLQ-C30) [Time Frame: Day 0, Day +28, |
| | Day +100, 6 months, 9 months, and 12 months] |
| | Overall response rate (ORR) of recipients compared across subgroups |
| | [Time Frame: Through 12-month follow-up]. Subgroups will be defined |
| | by degree of HLA-match from allogeneic donor; defined as the |
| | proportion of patients achieving complete remission (CR), complete |
| | remission with partial hematologic recovery (CRh), and complete |
| | remission with incomplete blood count recovery (CRi). Response will |
| | be assessed according to the criteria from the International Working |
| | Group Response Criteria |
| | Number of adverse events experienced by recipients compared across |
| | subgroups [Time Frame: Through Day +100]. Subgroups will be defined |
| | by degree of HLA-match from allogeneic donor; incidence, nature, and |
| | severity of adverse events tracked; adverse events will be collected from |
| | Day 0 to Day +35; however, bone marrow suppression (ANC < 500/μL) |
| | and adverse events of graft-versus-host disease (GVHD) involving the |
| | liver, skin, or gastrointestinal tract will be recorded until Day +100. |
| Inclusion | Refractory AML without CR after induction therapy (primary |
| Criteria | induction failure); relapsed AML after obtaining a CR; progressive AML |
| | after non-intensive therapy (e.g., HMA + venetoclax or targeted therapy); |
| | Intermediate risk to very-high-risk MDS by IPSS-R that is relapsed or |
| | refractory after prior therapy with an HMA-containing regimen |
| | At least 18 years of age. |
| | Available allogeneic donor that meets the following criteria: able and |
| | willing to undergo multiple rounds of leukapheresis; at least 18 years of |
| | age; in general good health, and medically able to tolerate leukapheresis |
| | required for harvesting the NK cells for this study; negative for hepatitis, |
| | HTLV, and HIV on donor viral screen; not pregnant; voluntary written |
| | consent to participate in this study; all HLA-match/mismatch statuses |
| | will be included, with preference for unmatched donors all else being |
| | equal |
| | Patients with known CNS involvement with AML are eligible |
| | provided that they have been treated and CSF is clear for at least 2 weeks |
| | prior to enrollment into the study. CNS therapy (chemotherapy or |
| | radiation) should continue as medically indicated during the study |
| | treatment. |
| | Karnofsky/Lansky performance status >50% |
| | Adequate organ function as defined as follows: total bilirubin <2 |
| | mg/dL; AST(SGOT)/ALT(SGPT) <3.0 × ULN; creatinine within normal |
| | institutional limits OR creatinine clearance ≥40 mL/min by Cockcroft- |
| | Gault Formula; oxygen saturation ≥90% on room air; ejection fraction ≥35%. |
| | Able to be off corticosteroids and any other immune suppressive |
| | medications beginning on Day −3 and continuing until 30 days after the |
| | last infusion of the WU-NK-101. However, use of low-level |
| | corticosteroids is permitted if deemed medically necessary. Low-level |
| | corticosteroid use is defined as 10 mg or less of prednisone (or equivalent |
| | for other steroids) per day. |
| | Women of childbearing potential must have a negative pregnancy test |
| | within 28 days prior to study registration. Female and male patients |
| | (along with their female partners) must agree to use two forms of |
| | acceptable contraception, including one barrier method, during |
| | participation in the study and until 30 days after the last WU-NK-101 |
| | infusion. |
| Exclusion | Relapsed after allogeneic transplantation. |
| Criteria | Circulating blast count >30,000/μL by morphology or flow cytometry |
| | (cytoreductive therapies including leukapheresis or hydroxyurea are |
| | allowed). |
| | Uncontrolled bacterial or viral infections, or known HIV, Hepatitis B |
| | or C infection. |
| | Uncontrolled angina, severe uncontrolled ventricular arrhythmias, or |
| | EKG suggestive of acute ischemia or active conduction system |
| | abnormalities. |
| | New progressive pulmonary infiltrates on screening chest x-ray or |
| | chest CT scan that have not been evaluated with bronchoscopy. |
| | Infiltrates attributed to infection must be stable/improving after 1 week |
| | of appropriate therapy (4 weeks for presumed or proven fungal |
| | infections). |
| | Known hypersensitivity to one or more of the study agents. |
| | Received any investigational drugs within the 14 days prior to the first |
| | dose of fludarabine. |
| | Pregnant and/or breastfeeding. |
| | Any condition that, in the opinion of the investigator, would prevent |
| | the participant from consenting to or participating in the study |

TABLE 24
CTP NCT01898793

| | |
|---|---|
| Study No. | NCT01898793 |
| Title | Cytokine-induced Memory-like NK Cells in Patients With Acute Myeloid |
| | Leukemia (AML) or Myelodysplastic Syndrome (MDS) |
| Summary/ | This phase I/2 trial studies the side effects and best dose of activated |
| Rationale | natural killer cells in treating patients with relapsed or refractory acute |
| | myeloid leukemia and myeloid dysplastic syndromes. Giving |
| | chemotherapy before a donor natural killer cell infusion helps stop the |
| | growth of cancer cells. It may also stop the patient's immune system from |
| | rejecting the donor's natural killer cells. Modified natural killer cells may |
| | help the body build an immune response to kill cancer cells. Aldesleukin |
| | (interleukin-2) may stimulate the white blood cells (including natural killer |
| | cells) to kill leukemia cells. |
| | In the phase II and pediatric portion of the study, the investigators intend to |
| | use maximal tolerated or tested (MT/TD) CIML NK cell dose as |
| | determined from the phase I part of this study. The phase II portion of the |
| | study also replaces IL-2 with ALT-803. The rationale for this change is to |
| | support the donor derived NK cells in vivo after adoptive transfer. |
| Conditions | Acute Myeloid Leukemia |
| | Myelodysplastic Syndrome |
| Study | Allocation: Non-Randomized |
| Design | Intervention Model: Sequential Assignment |
| | Masking: None (Open Label) |
| | Primary Purpose: Treatment |
| Interventions | Drugs: Fludarabine, Cyclophosphamide, IL-2 |
| | Biologicals: Cytokine-induced natural killer cells |
| | Procedure: Leukapheresis, peripheral blood for correlative studies, bone |
| | marrow for correlative studies |
| Schedule | Experimental: Phase I: 0.5 × 10\hat{ }6/kg CIML NK cells (Dose Levels 1-3) |
| | Lymphodepleting Preparative Regimen: Patients receive fludarabine |
| | phosphate IV over 1 hour on days −6 to −2 and cyclophosphamide IV over |
| | 2 hours on days −5 and −4. |
| | Donor Leukapheresis: Peripheral blood cells are collected from |
| | haploidentical related donors over 5 hours on day −1. |
| | CIML NK Cells: Patients undergo CIML NK cell infusion over 15-60 |
| | minutes on day 0. |
| | Interleukin-2: Patients receive aldesleukin SC every other day for 2 weeks |
| | starting on day 1 (total of 7 doses) |
| | Experimental: Pediatric Cohort: Maximum NK cell/number kg |
| | Lymphodepleting Preparative Regimen: Patients receive fludarabine |
| | phosphate IV over 1 hour on days −6 to −2 and cyclophosphamide IV over |
| | 2 hours on days −5 and −4. |
| | Donor Leukapheresis: Peripheral blood cells are collected from |
| | haploidentical related donors on Day −1 |
| | CIML NK Cells: Patients undergo CIML NK cell infusion on Day 0 |
| | Subcutaneous IL-2 will begin approximately 2-4 hours after infusion and |
| | will continue every other day through Day 12 for a total of 7 doses |
| | Experimental: Phase II (IL-2): Maximum NK cell/number kg |
| | The recipient will begin a lymphodepleting preparative regimen of |
| | fludarabine and cyclophosphamide on Day −6. The haploidentical donor |
| | identified by HLA matching of the immediate family members will |
| | undergo non-mobilized large volume (20-L) leukapheresis on Day −1, and |
| | the NK cell product will be infused into the recipient on Day 0. |
| | Subcutaneous IL-2 will begin approximately 2-4 hours after infusion and |
| | will continue every other day through Day 12 for a total of 7 doses. |
| | In each of the foregoing arms: peripheral blood for correlative studies will |
| | be collected at screening, Day 0 prior to CIML NK infusion, Days 1, 3, 7, |
| | 8, 10, 14, 21, 28, 42, 60, and 100, 6 months, 9 months, 12 months, and at |
| | disease relapse; and bone marrow for correlative studies will be collected |
| | at screening, Days 8, 14, and 28, between Day 42 and 60, at Day 100, and |
| | at disease relapse. |
| Primary | Maximal tolerated or tested dose (MT/TD) of CIML-NK cells (Phase I) |
| Outcome | [Time Frame: 35 days], defined as the dose level immediately below the |
| Measures | dose level at which 2 patients of a cohort (of 2 to 6 patients) experience |
| | dose-limiting toxicity (DLT) or the maximum dose if less than or equal to |
| | 1 patient suffers a DLT at the maximum dose. Summary statistics |
| | including proportions and their 95% confidence interval will be calculated. |
| | Non-Hematologic dose limiting toxicities (DLT) are defined as any |
| | CTCAE grade 3 or higher non-hematologic adverse event considered |
| | possibly, probably, or definitely related to CIML NK cell infusion. Non- |
| | clinically significant metabolic adverse events will not be considered DLTs |
| | regardless of CTCAE grade. Hematologic dose limiting toxicity is defined |
| | as failure to recover hematopoiesis (ANC ≥ 500/μL, platelet |
| | count ≥50,000/uL) by Day 35 post-CIML NK cell infusion. Hematologic AEs |
| | related to persistent disease/disease relapse or other causes will not be |
| | considered DLTs. |
| | Complete remission rate (CR/CRi) in participants with relapsed or |
| | refractory AML following CIML NK therapy (Phase II) [Time Frame: Up |
| | to 3 years]. Complete remission rate (CR): Morphologically leukemia free |
| | state (i.e. bone marrow with <5% blasts by morphologic criteria and no |
| | blasts with Auer rods, no evidence of extramedullary leukemia) and |
| | absolute neutrophil count ≥1000/μL and platelets ≥100,000/μL. Patient |
| | must be independent of transfusions. Complete Remission with |
| | Incomplete Blood Count Recovery (CRi): All of the above criteria for CR |
| | must be met, except that absolute neutrophils <1000/μL or |
| | platelets <100,000/μL in the blood. |
| | Safety of CIML NK cells (Pediatric) as measured by the frequency and |
| | incidence of adverse events [Time Frame: Through Day 100], graded using |
| | the National Cancer (NCI) Common Terminology Criteria for Adverse |
| | Events (CTCAE) version 4.0. |
| | AEs will be collected from Day 0 to Day +35; |
| | however, bone marrow suppression (ANC < 500/uL/μL) and AEs of |
| | GVHD involving the liver, skin, or GI tract will be recorded to Day 100. |
| Secondary | Response assessed according to IWG criteria (Phase 1, Phase II, and |
| Outcome | Pediatric) [Time Frame: 35 days], reported with 95% confidence intervals. |
| Measures | Duration of remission (DOR) (Phase I, Phase II, and Pediatric) [Time |
| | Frame: Up to 3 years], defined only for patients who achieve a CR or PR, |
| | and is measured from the first date of attaining CR or PR until the date of |
| | disease progression or death. |
| | Time to progression (Phase I, Phase II, and Pediatric) [Time Frame: Up |
| | to 3 years], defined as the time from date of first dose of fludarabine until |
| | evidence of disease progression. |
| | Disease free survival (DFS) (Phase I, Phase II, and Pediatric) [Time |
| | Frame: Up to 3 years], defined as the time from the day CR or CRi is |
| | documented until disease progression or death. |
| | Overall survival (OS) (Phase I, Phase II, and Pediatric) [Time Frame: Up |
| | to 3 years], defined from the date of first dose of fludarabine on this study |
| | until death. |
| | Toxicity as measured by the frequency and incidence of serious adverse |
| | events (Phase I and Phase II) [Time Frame: Through Day 100], graded |
| | using the National Cancer (NCI) Common Terminology Criteria for |
| | Adverse Events (CTCAE) version 4.0. AEs will be collected from Day 0 to |
| | Day +35; however, bone marrow suppression (ANC < 500/uL/μL) and |
| | AEs of GVHD involving the liver, skin, or GI tract will be recorded to Day |
| | 100. |
| Inclusion | Diagnosis requirement for phase I patients: |
| Criteria | Refractory AML without complete remission (CR) after induction |
| | therapy (primary induction failure) or relapsed AML after obtaining a CR; |
| | or high-risk AML (by ELN criteria; See Appendix C) in complete |
| | remission (CR) and has either refused hematopoietic stem cell |
| | transplantation, or is currently not eligible for hematopoietic stem cell |
| | transplantation, or for whom hematopoietic stem cell transplantation is |
| | being reserved for later relapse (this is inclusive of patients with minimal |
| | residual disease evidenced by cytogenetics, molecular testing, and/or flow |
| | cytometry); or Myelodysplastic syndrome (MDS) with excess blasts (>5%) |
| | and progressive disease at any time after initiation of DNA hypomethylator |
| | treatment during the past 2 years, OR failure to achieve complete or partial |
| | response or hematological improvement (see section 12.4) after at least six |
| | cycles of azacytidine or four cycles of decitabine administered during the |
| | past 2 years, OR intolerance to azacytidine or decitabine. MDS patients |
| | with isolated 5q- abnormalities that meet these criteria after lenalidomide |
| | therapy and DNA hypomethylator therapy are also eligible. |
| | Diagnosis requirement for phase II patients: |
| | Refractory AML without CR after induction therapy (primary induction |
| | failure) or relapsed AML after obtaining a CR. Favorable-risk core binding |
| | factor (CBF) mutated AML and acute promyelocytic leukemia (APL) will |
| | be excluded. |
| | Diagnosis requirement for pediatric cohort patients: |
| | Refractory AML without complete remission (CR) after induction |
| | therapy (primary induction failure) or relapsed AML after obtaining a CR. |
| | Age requirement for phase I and phase II patients: At least 18 years of |
| | age. |
| | Age requirement for pediatric cohort: 2-17 years of age. |
| | Available HLA-haploidentical donor that meets the following criteria: |
| | related donor (parent, sibling, offspring, or offspring of sibling); 2) at least |
| | 18 years of age; HLA-haploidentical donor/recipient match by at least |
| | Class I serologic typing at the A&B locus; in general good health, and |
| | medically able to tolerate leukapheresis required for harvesting the NK |
| | cells for this study; negative for hepatitis, HTLV, and HIV on donor viral |
| | screen; not pregnant; and voluntary written consent to participate. |
| | Patients with known CNS involvement with AML are eligible provided |
| | that they have been treated and CSF is clear for at least 2 weeks prior to |
| | enrollment into the study. CNS therapy (chemotherapy or radiation) should |
| | continue as medically indicated during the study treatment. |
| | Karnofsky/Lansky performance status ≥50% |
| | Adequate organ function as defined as follows: total bilirubin ≤2 mg/dL; |
| | AST(SGOT)/ALT(SGPT) ≤3.0 × IULN; creatinine within normal |
| | institutional limits OR creatinine clearance ≥50 mL/min/1.73 m2 by |
| | Cockcroft-Gault Formula (adults) or Schwartz formula (pediatric cohort); |
| | oxygen saturation ≥90% on room air; ejection fraction ≥35%. |
| | Able to be off corticosteroids and any other immune suppressive |
| | medications beginning on Day −3 and continuing until 30 days after the |
| | infusion of the CIML NK cells. However, use of low-level corticosteroids |
| | is permitted if deemed medically necessary. Low-level corticosteroid use is |
| | defined as 10 mg or less of prednisone (or equivalent for other steroids) per |
| | day. |
| | Women of childbearing potential must have a negative pregnancy test |
| | within 28 days prior to study registration. Female and male patients (along |
| | with their female partners) must agree to use two forms of acceptable |
| | contraception, including one barrier method, during participation in the |
| | study and throughout the DLT evaluation period. |
| Exclusion | Relapsed after allogeneic transplantation. |
| Criteria | Isolated extramedullary relapse (phase II only). |
| | More than one course of salvage chemotherapy for primary induction |
| | failure or AML relapsing after CR1 (phase II only). |
| | Circulating blast count ≥30,000/μL by morphology or flow cytometry |
| | (cytoreductive therapies including leukapheresis or hydroxyurea are |
| | allowed). |
| | Uncontrolled bacterial or viral infections, or known HIV, Hepatitis B or |
| | C infection. |
| | Uncontrolled angina, severe uncontrolled ventricular arrhythmias, or |
| | EKG suggestive of acute ischemia or active conduction system |
| | abnormalities. |
| | New progressive pulmonary infiltrates on screening chest x-ray or chest |
| | CT scan that have not been evaluated with bronchoscopy. Infiltrates |
| | attributed to infection must be stable/ improving after 1 week of |
| | appropriate therapy (4 weeks for presumed or proven fungal infections). |
| | Known hypersensitivity to one or more of the study agents. |
| | Received any investigational drugs within the 14 days prior to the first |
| | dose of fludarabine. |
| | Pregnant and/or breastfeeding. |

Memory NK cells which have been expanded then primed, or expanded and primed concurrently, according to the methods disclosed herein, are expected to be effective in the treatment of AML, MDS, and other diseases, for example as shown in the clinical trial protocols above.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

1. A population of purified cytokine-induced memory-like natural killer cells (CIML NK cells) produced by, sequentially:
a) expanding purified NK cells; and
b) priming the NK cells.

2. (canceled)

3. The memory NK cells according to claim 1, wherein the NK cells are enriched from fresh or frozen leukapheresate or donor blood.

4. The memory NK cells according to claim 1, wherein the NK cells are differentiated from lymphoid progenitor cells.

5. The memory NK cells according to claim 1, wherein the NK cells are purified by negative or positive selection, or combinations thereof.

6. The memory NK cells according to claim 1, wherein the NK cells are primed by exposure to:
one or more of IL-12, IL-23, IL-27, and IL-35;
one or more of IL-2, IL-4, IL-7, IL-9, IL-15, and IL-21; and
one or more of IL-18, IL-1a, IL-1b, IL-36a, IL-36b, and IL-36g;
or functional fragments thereof, and/or fusion proteins comprising functional fragments thereof, or a combination of any of the foregoing.

7. The memory NK cells according to claim 6, wherein the NK cells are primed by exposure to 18t15-12s.

8. (canceled)

9. The memory NK cells according to claim 6, wherein the NK cells are primed by exposure to IL-12, IL-15, and IL-18.

10. (canceled)

11. The memory NK cells according to claim 1, wherein the NK cells are expanded by exposure to 7t15-21s and ATF1.

12. (canceled)

13. (canceled)

14. (canceled)

15. (canceled)

16. (canceled)

17. (canceled)

18. (canceled)

19. (canceled)

20. (canceled)

21. The memory NK cells according to claim 1, additionally comprising at least one chimeric antigen receptor (CAR), comprising:
a) at least one extracellular ligand-binding domain targeting an antigen on a target cell;
b) a hinge domain;
c) a transmembrane domain;
d) optionally, one or more co-stimulatory domains; and
e) a cytoplasmic signaling domain.

22. A method of making memory NK cells comprising:
a) purifying an enriched population of NK cells;
b) expanding the NK cells; and
c) priming the NK cells.

23. (canceled)

24. (canceled)

25. (canceled)

26. (canceled)

27. The method according to claim 22, wherein the NK cells are primed by exposure to.
one or more of IL-12, IL-23, IL-27, and IL-35;
one or more of IL-2, IL-4, IL-7, IL-9, IL-15, and IL-21; and
one or more of IL-18, IL-1a, IL-1b, IL-36a, IL-36b, and IL-36g;
or functional fragments thereof, and/or fusion proteins comprising functional fragments thereof, or a combination of any of the foregoing.

28. The method according to claim 27, wherein the NK cells are primed by exposure to 18t15-12s.

29. (canceled)

30. The method according to claim 27, wherein the NK cells are primed by exposure to IL-12, IL-15, and IL-18.

31. (canceled)

32. The method according to claim 22, wherein the NK cells are expanded by exposure to 7t15-21s and ATF1.

33. (canceled)

34. (canceled)

35. (canceled)

36. (canceled)

37. (canceled)

38. (canceled)

39. (canceled)

40. (canceled)

41. (canceled)

42. A method of treating a proliferative malignancy, the method comprising administration of the memory NK cells according to claim 1, to a patient in need thereof.

43. The method of claim 42, wherein the cells are administered fresh to patients.

44. The method of claim 42, wherein the proliferative malignancy is a cancer.

45. The method of claim 44, wherein the cancer is hematologic.

46. (canceled)

47. (canceled)

48. (canceled)

49. The method of claim 45, wherein the hematologic cancer is a T-cell lymphoma.

50. The method of claim 49, wherein the T-cell lymphoma is chosen from T-cell acute lymphoblastic leukemia/lymphoma (T-ALL), peripheral T-cell lymphoma (PTCL), T-cell chronic lymphocytic leukemia (T-CLL), and Sezary syndrome.

51. (canceled)

52. (canceled)

53. (canceled)

54. (canceled)

55. (canceled)

56. (canceled)

57. (canceled)
